(19) United States
(12) Patent Application Publication
(10) Pub. No.: US 2026/0327202 A1
(43) Pub. Date: Oct. 22, 2026

(54) COMPOUND CONTAINING 2-METHYL-2-(3-(PIPERIDIN-3-YL)PHENOXY)-2-METHYLPROPANAMIDE SKELETON

(71) Applicant: JIANGSU MINGSHENG JUTAI BIOTECHNOLOGY CO. LTD, Nantong, Jiangsu (CN)

(72) Inventor: Wenhua ZHU, Nantong, Jiangsu (CN)

(21) Appl. No.: 19/145,990

(22) PCT Filed: Jan. 5, 2024
(86) PCT No.: PCT/CN2024/070917

§ 371 Date: Jun. 24, 2026

(30) Foreign Application Priority Data
Jan. 5, 2023 (CN) 202310015931.8
Jan. 5, 2023 (CN) 202310015933.7

(51) Int. Cl. C07D 401/12; A61K 31/451; A61K 31/454; A61P 35/00; C07D 211/96; C07D 401/14

(52) U.S. Cl. CPC C07D 401/12; A61K 31/451; A61K 31/454; A61P 35/00; C07D 211/96; C07D 401/14; C07B 2200/13

(57) ABSTRACT

Provided in the present invention is a compound containing a 2-methyl-2-(3-(piperidin-3-yl)phenoxy)-2-methylpropanamide skeleton. Specifically, provided in the present invention is a compound of formula I or formula II, or a pharmaceutically acceptable salt thereof. The compound of formula I or formula II of the present invention has an excellent ability to inhibit BCL9/β-catenin interaction.

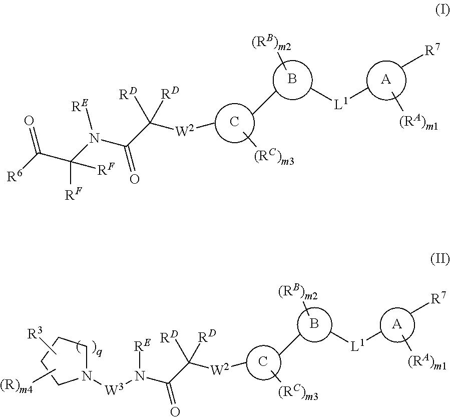

### TECHNICAL FIELD

The present invention belongs to the pharmaceutical field, specifically relating to a compound containing a 2-methyl-2-(3-(piperidine-3-yl)phenoxy)-2-methylpropamide backbone and its use in the interaction of BCL9 (B-cell lymphoma 9)/β-catenin targeting it.

### BACKGROUND TECHNOLOGY

Wnt/β-catenin signaling is critical for normal embryonic development and throughout life. In addition, abnormal Wnt signaling is associated with various diseases, especially cancer. Recent studies have shown that direct targeting of β-catenin/B-cell lymphoma 9 (BCL9) protein-protein interactions (PPIs) is a promising strategy for blocking the Wnt pathway. The discovery process of its inhibitors has been facilitated by advances in the understanding of the eutectic complexes and mechanisms of action of β-catenin/BCL9 interactions, but only a few inhibitors have been reported.

Classical Wnt signaling is a highly conserved developmental signaling pathway that regulates cell proliferation, differentiation, and survival. β-catenin is often considered a key effector of Wnt signaling. In the absence of Wnt monoselectivity (Wntoff), the cytoplasmic pool of β-catenin binds to glycogen synthase kinase 3β (GSK3β), casein kinase 1α (CK1α), scaffold protein AXIN, and tumor suppressor adenomatous polyposis Escherichia coli (APC) to regulate phosphorylation. Subsequently β catenin is degraded by proteasomes. β-catenin recruits co-activators, including BCL9 or B-cell lymphoma 9-like (B9L), Pygo, CREB-binding protein (CBP), etc., to promote cell proliferationTranscription of migratory and viable genes such as cyclin D1, c-myc, viability protein, and LEF1. The occurrence and progression of many types of cancer are closely related to these Wnt target genes, including colorectal, breast, lung, hepatocellular carcinoma, leukemia, and multiple myeloma.

Advances using the β-catenin/BCL9 complex follow advances in reliable biochemical assays and drug discovery strategies to provide further understanding of interactions that may lead to the discovery of new anti-cancer drugs. To date, several different types of β-catenin/BCL9PPI inhibitors have been reported. It can be mainly divided into two categories: peptide inhibitors and non-peptide small molecule inhibitors. However, the exploration of β-catenin/BCL9PPI inhibitors, especially non-peptide small molecule inhibitors, is still in the primary research exploration stage.

In summary, there is an urgent need to develop a small molecule compound with high target affinity and good cell membrane permeability to target BCL9 (B-cell lymphoma 9))/β-catenin interaction.

### INVENTION CONTENT

The purpose of the present invention is to provide a new class of small molecule compounds targeting the interaction of BCL9/β-catenin.

In the first aspect of the present invention, a compound or a pharmaceutically acceptable salt thereof, or its isomer, solvent complex, crystal form, or prodrug, is provided, and the compound is shown in Formula I

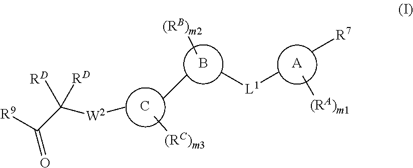

thereinto
R⁹ is

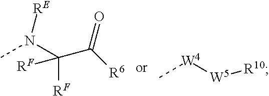

W⁴ and W⁵ are independently selected from the following groups: None, —O—, —S—, —C(O)—, —S(O)—, —S(O)₂—, —N(R^{s1})—, —C(R^{s2})₂—;
R¹⁰ is H, OH, R⁶, or a group that is unsubstituted or replaced by one or more R^{H} and is selected from the following groups: C₁₋₆ alkyl groups, C₃₋₁₀ naphthenic alkyl groups, 4 to 10 members heterocyclic alkyl groups, C₃₋₁₀ cyclic alkyl groups, 4 to 10 members heterocyclic alkyl groups, C₆₋₁₀ aryl groups, and 5 to 10 members heteroaryl groups;
R^{H} is selected from the following group: R, optionally substituted C₁₋₆ halogenated alkyl groups;
R^{E} is selected from the following groups: H, optionally substituted C₁₋₄ alkyl groups, optionally substituted C₃₋₁₀ naphthenic alkyl groups, optionally substituted 4 to 10 membered heteronaphthenic alkyl groups;
R^{F} is independently selected from the following groups: H, optionally substituted C₁₋₄ alkyl groups, optionally substituted C₃₋₁₀ naphthenic alkyl groups, optionally substituted 4 to 10 membered heteronaphthenic alkyl groups; or two R^{F}R^{F}S and carbon atoms connected to them together form an optionally substituted C₃₋₆ naphthenic group or an optionally substituted 4 to 6-membered heterocyclic group;
R⁶ is optionally substituted groups selected from the following groups: —OR², C3-12 naphthenic groups, 4 to 10-membered heterocyclic alkyl groups connected to the rest by carbon atoms on the ring, and —NR⁴R⁵;
R² is selected from the following groups: H, optionally substituted C₁₋₆ alkyl group, optionally substituted C₃₋₁₀ naphthenic alkyl group, optionally substituted 4 to 8-membered heteronaphnolyl group, optionally substituted C₆₋₁₀ aryl group, optionally substituted 5 to 10-membered heteroaryl group, optionally substituted C₃₋₁₀ cyclic alkyl group, optionally substituted 4 to 10-membered heterocyclic alkyl group;
R⁴ and R⁵ are each selected groups from the following groups that are optionally replaced or replaced by one or more (e.g., 1, 2, or 3) R³s: H, C₁₋₆ alkyls, C₃₋₁₀ naphthenic alkyls, 4 to 8-membered heterocyclic alkyl groups, C₆₋₁₀ aryl groups, 5 to 10-member heteroaryl groups, C₃₋₁₀ cyclene groups, and 4 to 10-member heterocyclic alkyl groups; Alternatively, R⁴ and R⁵ bind to their attached nitrogen atoms to form an optionally substituted or selected ring from the following group of R³s that are substituted by one or more (e.g., 1, 2, or 3) R³s: 4 to 10 heterocyclic alkyl groups, 4 to 10 members heterocyclic alkyl groups, or 5 to 10 member heteroaryl groups;
R³ are independently selected from the following groups: R, —OR³¹, —C₁₋₄ alky-OR³¹, —N(R³²)R³³, —C₁₋₄ alky-N(R³¹)R³²;
R³¹ is selected from the following groups: H, optionally substituted C₁₋₄ alkyl groups, R³⁴, —C₁₋₄ alkyl —R³⁴;
R³² is selected from the following group: H, optionally substituted C₁₋₄ alkyl groups;
R³³ is selected from the following groups: H, optionally substituted C₁₋₄ alkyl groups, R³⁴, —C₁₋₄ alkyl —R³⁴;
R³⁴ is selected from the following groups: C₃₋₁₀ naphthenyl, 4 to 8 members heterocyclic alkyl groups, C₆₋₁₀ aryl groups, 5 to 10 membered heteroaryl groups, C₃₋₁₀ cyclene groups, 4 to 10 membered heterocyclic alkyl groups; wherein, the naphthenic alkyl group, the aryl group, the heteroaryl group, the cyclic alkene group and the heterocyclic alkyl group are optionally replaced by one or more groups selected from the following group: —NH2, R;
R^{D} is independently selected from the following groups: H, optionally substituted C₁₋₄ alkyl groups; Alternatively, the two R^{D}s and the carbon atoms attached to them together form groups selected from the following groups: optionally substituted C₃₋₁₀ naphthenic alkyl groups, optionally substituted 4 to 10-membered heteronaphthenic alkyl groups;
W² is selected from the following group: —O—, —S—, —N(R^{s1})—;
Ring C is optionally replaced by the following groups: C₆₋₁₀ aryl groups, and 5 to 10 meta heteroaryl groups;
m3=0, 1, 2, 3 or 4;
Each R^{C} is R^{C1} or R^{s1} independently;
Each R^{C1} is independently selected from the following groups: halogens, optionally substituted C₁₋₆ alkyl groups, optionally substituted C₁₋₆ halogenated alkyl groups, hydroxyl groups and optionally substituted C₁₋₆ alkoxy groups, and optionally substituted C₁₋₆ halogenated alkoxy groups;
Ring B is the ring selected from the following group of alternatives: C3-12 naphthenic alkyl group, 4 to 12 metaphthenic alkyl groups, C₃₋₁₀ cyclene groups, 4 to 10 membered heteronaphthenic alkyl groups;
Each R^{B} is R^{B1}, R^{s1} or R^{s2} independently;
Each R^{B1} is independently selected from the following groups: halogens, hydroxyl groups, cyanos, optionally substituted C₁₋₆ alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₃₋₁₀ cyclic alkyl groups, optionally substituted 4 to 10-membered heterocyclic alkyl groups, optionally substituted C₆₋₁₀ aryl groups, and optionally substituted 5 to 10-membered heteroaryl groups;
m2=0, 1, 2, 3 or 4;
L¹ is —X—(W¹)ₙ₁—;
X is selected from the following groups: —C(O)—, —S(O)— and —S(O)₂—;
Each W¹ is independently selected from the following groups: none, —O—, —S—, —C(O)—, —S(O)—, —S(O)₂—, —N(R¹)—, —N(R^{s1})—, —CH(R⁸)—, —C(R^{s2})₂—;
subscript n1=0, 1, 2 or 3;
Ring A is optionally replaced by the following group: C₆₋₁₀ aryl; 5 to 10 membered heteroaryl groups; C₆₋₁₀ aryl groups substituted by C₃₋₁₀ naphthenic alkyl groups, 4 to 10 meta heterocyclic alkyl groups, C₃₋₁₀ cyclene groups, 4 to 10 membered heterocyclic alkyl groups, C₆₋₁₀ aryl groups or 5 to 10 membered heteroaryl groups; 5 to 10 membered heteroaryl groups replaced by C₃₋₁₀ naphthenic alkyl groups, 4 to 10 metaphnocyclic alkyl groups, C3-10 cyclene groups, 4 to 10 membered heterocyclic alkyl groups, C₆₋₁₀ aryl groups or 5 to 10 membered heteroaryl groups; C₆₋₁₀ aryl fused with C₃₋₁₀ naphthenyl, 4 to 10 heteronaphthenic alkyl groups, C3-10 cyclene groups, 4 to 10 members heterocyclic alkenyl groups, C₆₋₁₀ aryl groups or 5 to 10 member heteroaryl groups; 5 to 10 membered heteroaryl groups fused with C₃₋₁₀ naphthenyl, 4 to 10 metaphthenic alkyl groups, C₃₋₁₀ cyclene groups, 4 to 10 membered heterocyclic alkyl groups, C₆₋₁₀ aryl groups, or 5 to 10 membered heteroaryl groups;
m1=0, 1, 2, 3 or 4;
Each R^{A} is R^{A1}, R^{s1} or R^{s2} independently;
Each R^{A1} is independently selected from the following groups: halogens, optionally substituted C₁₋₆ alkyl groups, optionally substituted C₁₋₆ halogenated alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₆₋₁₀ aryl groups, optionally replaced 5 to 10 membered heteroaryl groups; Alternatively, when two R^{A}R^{A1}s are located on adjacent ring atoms, the two R^{A}R^{A1}s and their adjacent ring atoms together form a selected ring from the following group: optionally substituted C₃₋₁₀ naphthenic alkyl group, optionally substituted 4 to 10-membered heterocyclic alkyl group, optionally substituted C₃₋₁₀ cyclic alkyl group, optionally substituted 4 to 10-membered heterocyclic alkene group, optionally substituted C₆₋₁₀ aryl group, optionally substituted C₆₋₁₀ aryl group, optionally replace 5 to 10 membered heteroaryl groups;
Each R¹ and R⁸ are independently selected from the following groups: H, optionally substituted C₁₋₆ alkyl groups, optionally substituted C₃₋₆ naphthenic alkyl groups, halogens, optionally substituted C₁₋₆ halogenated alkyl groups, optionally substituted C₁₋₆ halogenated alkoxy groups (—O—C₁₋₆ halogenated alkyl groups), optionally substituted C₁₋₆ alkyl groups-O—C₁₋₆ alkyl groups, optionally substituted C₁₋₆ halogenated alkyl groups, Optionally substituted C₁₋₆ halogenated alkyl group-S—C₁₋₆ alkyl group, optionally substituted C₁₋₆ aminoalkyl group, optionally substituted C₃₋₁₀ naphthenic alkyl group, optionally substituted 4 to 10-membered heterocyclic alkyl group, optionally substituted C₆₋₁₀ aryl group, optionally substituted 5 to 10-membered heteroaryl group, optionally substituted C₃₋₁₀ halocyclic alkyl group, optionally substituted C₃₋₁₀ naphthenic alkyl group, optionally substituted 4 to 10-membered heterocyclic alkyl-C₁₋₄ alkyl groups, optionally substituted C₆₋₁₀ aryl-C₁₋₄ alkyl groups, optionally substituted 5 to 10-membered heteroaryl-C₁₋₄ alkyl groups, optionally substituted C₃₋₁₀ cyclic alkyl groups-C₁₋₄ alkyl groups, optionally substituted 4 to 10-membered heterocyclic alkyl groups-C₁₋₄ alkyl groups; Alternatively, R¹ or R⁸ and R^{s1} or R^{s2} on ring A form optionally substituted C₄₋₁₀ naphthenic or 4-10 heterocyclic alkyl groups;
R⁷ is optionally substituted groups from the following groups: none, C₁₋₆ alkyl groups, C₃₋₁₀ naphthenyl groups, 4 to 10 members heterocyclic alkyl groups, C₃₋₁₀ cyclic alkyl groups, 4 to 10 members heterocyclic alkyl groups, C₆₋₁₀ aryl groups, and 5 to 10 member heteroaryl groups.

R^{s1} is independently selected from the following groups: H, optionally substituted C₁₋₄ alkyl group, optionally substituted C₃₋₆ naphthenic group, and optionally substituted 4 to 6-membered heterocyclic group;
R^{s2} are independently selected from the following groups: H, optionally substituted C₁₋₄ alkyl groups, optionally substituted C₃₋₆ naphthenic alkyl groups, and optionally substituted 4 to 6-membered heterocyclic groups; or two R^{s2} and the carbon atoms attached to them together form an optionally substituted C₃₋₆ naphthenic group or an optionally substituted 4 to 6-membered heterocyclic group;
Unless otherwise defined, said optional substitution means an unsubstituted or group of one or more (e.g., 1, 2, 3, or 4) hydrogen substituted by an R substituent;
R are independently selected from the following groups: D, halogens, C₁₋₆ alkyls, C₁₋₆ halogenated alkyls, C₁₋₆ hydroxyalkyls, C₂₋₆ alkyl groups, C₂₋₆ alkyl groups, —CN, —OR′, —NO2, —NR′R″, —SR′, OC(O)R′, —C(O)R′, —CO2R′, —CONR′, —OC(O)NR′R″, —NR″C(O)R′, —NR″—C(O)NR′R″, —NR′C (O)₂R′, —S(O)R′, —S(O)₂R′, —S(O)₂NR′R″, NR″S(O)₂R′, any C₃₋₁₀ naphthenic alkyl group replaced by one or more R′, any 4 to 10 member heterocycloalkyl group replaced by one or more R′, any C₆₋₁₀ aryl group replaced by one or more R′, any 5 to 10 member heteroaryl group replaced by one or more R′, any —C₁₋₄ alkyl group C₃₋₁₀ naphthenic group replaced by one or more R″, any —C₁₋₄ alkyl group replaced by one or more R″, choose —C₁₋₄ alkyl-C₆₋₁₀ aryl group replaced by one or more R″″, choose —C₁₋₄ alkyl-5 to 10 aryl group replaced by one or more R″s;
Each R′ is independently selected from the following groups: H, D, C₁₋₆ alkyls, C₁₋₆ halogenated alkyls, any C₃₋₁₀ naphthenic alkyl group replaced by one or more R″s, any 4 to 10 heterocyclic alkyl group replaced by one or more R″s, any C₆₋₁₀ aryl group replaced by one or more R″s, any 5 to 10 membered heteroaryl groups replaced by one or more R″s, any —C₁₋₄ alkyl group replaced by one or more R′s-C₃₋₁₀ naphthenic alkyl groups, choose —C₁₋₄ alkyl-4 to 10-membered heterocyclic alkyl groups replaced by one or more R″″, choose —C₁₋₄ alkyl —C₆₋₁₀ aryl group replaced by one or more R″, choose —C₁₋₄ alkyl-5 to 10-member heteroaryl group replaced by one or more R″s;
Each R″ is selected from the following groups: H, D, C₁₋₄ alkyls, C₁₋₄ halogenated alkyls, and C₃₋₄ naphthenic groups;
Each R″ is independently selected from the following groups: D, halogen, hydroxyl group, nitro, CN, C₁₋₆ alkyl group, C₁₋₆ halogenated alkyl group.

In another preferred example, the compound is shown by Formula I-1 or Formula I-2

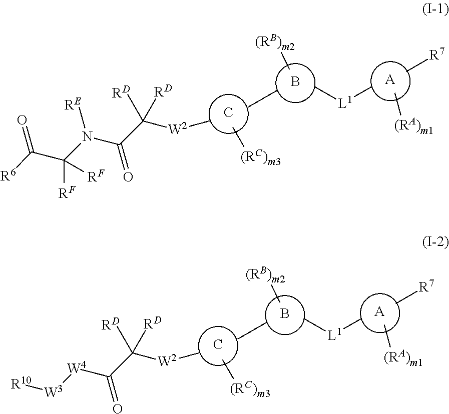

In another preferred example, R⁷ is an optional substitute group from the following groups: C₁₋₆ alkyl groups, C₃₋₁₀ naphthenic alkyl groups, 4 to 10 heterocyclic alkyl groups, C₃₋₁₀ cyclic alkyl groups, 4 to 10 member heterocyclic alkyl groups, C₁₋₁₀ aryl groups, and 5 to 10 member heteroaryl groups.

In another preferred example, R⁷ is an optional substitute for groups selected from the following groups: C₆₋₁₀ aryl groups, and 5 to 10 mixed aryl groups.

In another preferred example, in R⁷, the heteroaryl group comprises 1, 2 or 3 nitrogen heteroatoms as ring atoms, and the remaining ring atoms in the heteroaryl group are carbon atoms.

In another preferred example, R⁷ is optionally replaced by groups selected from the following groups:

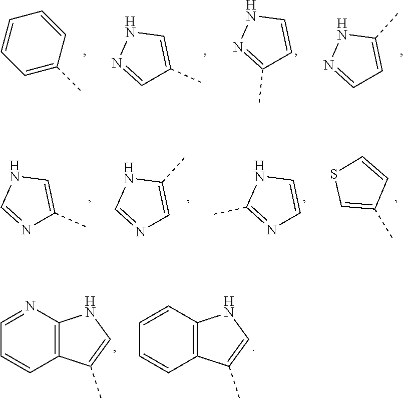

In another preferred example, R⁷ is an optionally substituted phenyl or 5-membered heteroaryl group.

In another preferred example, R⁷ is optionally replaced by groups selected from the following groups:

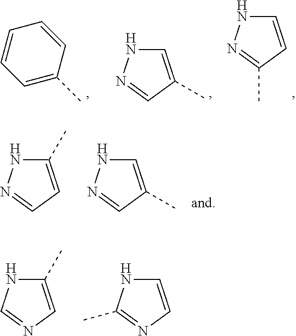

In another preferred example, R⁷, the optional substitution refers to the unsubstituted or group where 1 or 2 hydrogens in the group are replaced by the selected R substituent.

In another preferred example, R⁷ is selected from the following group:

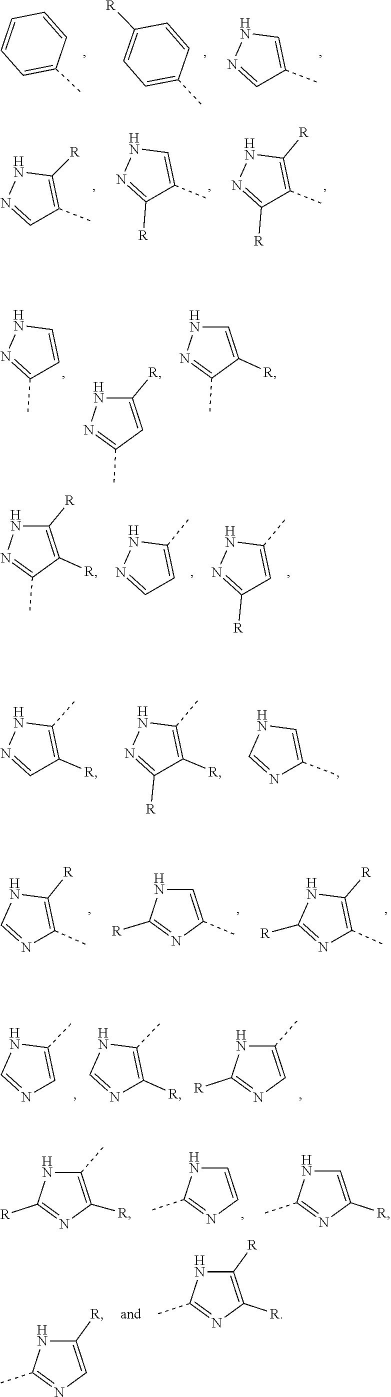

In another preferred example, in R⁷, R is independently selected from the following groups: C₁₋₆ alkyl group, —NR′R″; wherein each R′ is independently selected from the following groups: H, C₁₋₆ alkyl groups; Each R″ is selected from the following group:

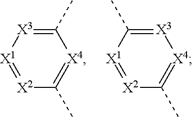

In another preferred example, ring A is the ring selected from the following group:
wherein X¹, X², X³ and X⁴ are independently selected from the following groups: CH and N.

In another preferred example, ring A is:

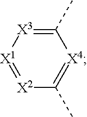

wherein X¹, X², X³ and X⁴ are independently selected from the following groups: CH and N.

In another preferred example, up to 1 or 2 of X¹, X², X³, and X⁴ are N.

In another preferred example, X¹, X², X³, and X⁴ are all CH.

In another preferred example, m1=0, 1, or 2; Better, m1=0 or 1; Better, m=0.

In another preferred case, the R^{A}s are R^{s1} or R^{A}R^{A1} independently; And R^{A}R^{A1} is selected from the following groups: halogens, optionally substituted C₁₋₆ alkyl groups, optionally substituted C₁₋₆ halogenated alkyl groups, and optionally substituted C₁₋₆ alkoxy groups (preferably, R^{A}R^{A1} is halogen).

In another preferred case, R^{A}s are H, C₁₋₄, or R^{A}R^{A1} independently; And R^{A}R^{A1} is selected from the following groups: halogens, optionally substituted C₁₋₆ alkyl groups, optionally substituted C₁₋₆ halogenated alkyl groups, and optionally substituted C₁₋₆ alkoxy groups (preferably, R^{A}R^{A1} is halogen).

In another preferred case, n1=0, 1, or 2. In another preferred case, each W¹ is independently —C(R^{s2})₂— (preferably, —CH2-).

In another preferred case, n1=0.

In another preferred case, L¹ is —X—: Better, L¹ is —C(O)— or —SO₂—. In another preferred example, L¹ is —SO₂—.

In another preferred example, ring B is optionally replaced by the following groups: C₃₋₁₂ naphthenic alkyl groups, 4 to 12-member heterocyclic alkyl groups.

In another preferred example, ring B is

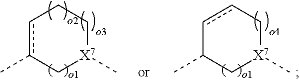

wherein - it is a single or double bond, X₇ is N or CH, o1 is 1 or 2, o2 is 0, 1, 2 or 3, o3 is 0 or 1, and o4 is 0, 1, 2 or 3.

In another preferred example, X⁷ is N.

In another preferred example, o1 is 1.

In another preferred case, when X⁷ is N, N in ring B is connected to L¹.

In another preferred example, ring B is

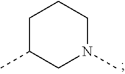

Preferably, N in ring B is connected to L¹.

In another preferred example, ring B is

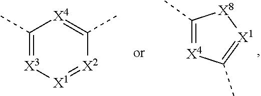

where X¹, X², X³, and X⁴ are selected independently from the following groups: CH, N, and X⁸ are S, O, or NH.

In another preferred case, R^{B}s are either R^{s1} or R^{s2}.

In another preferred example, m2=0.

In another preferred example,

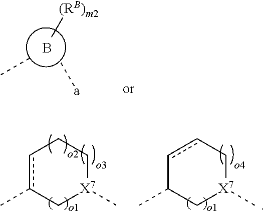

In another preferred example,

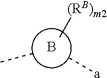

it is

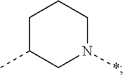

where * refers to the connection to L¹.

In another preferred example, ring C is

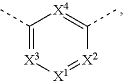

where X¹, X², X³ and X⁴ are independently selected from the following groups: CH and N.

In another preferred example, in ring C, up to 1 or 2 of X¹, X², X³, and X⁴ are N.

In another preferred example, ring C is

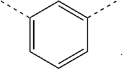

In another preferred example, m3=0. In another preferred example, m3=1, 2, 3, or 4.

In another preferred example, R^{C1} is selected from the following groups: halogens (preferably F, Cl), C₁₋₆ halogenated alkyl groups (preferably trifluoromethyl), and C₁₋₆ alkoxy groups (preferably methoxy).

In another preferred example, ring C is;

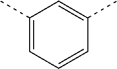

m3=0, 1 or 2; R^{C} is H or R^{C1}; R^{C1} is selected from the following groups: halogens (preferably F, Cl), C₁₋₆ halogenated alkyl groups (preferably trifluoromethyl), and C₁₋₆ alkoxyl groups (preferably methoxy groups); Better, R^{C1} is halogen.

In another preferred example, W² is —O— or —N(R^{s1})—; Preferably, W² is —O— or —NH—.

In another preferred example, W² is —O—.

In another preferred example, R^{D}s are selected independently from the following groups: H, optionally substituted C₁₋₄ alkyl groups; Alternatively, the two R^{D}s and the carbon atoms attached to them work together to form an optionally substituted C₃₋₁₀ naphthenic group.

In another preferred example, R^{D}s are selected independently from the following groups: H, optionally substituted C₁₋₄ alkyl groups.

In another preferred case, R^{D} is selected independently from the following groups: H, methyl, and ethyl.

In another preferred case, R^{D} is both methyl groups.

In another preferred example,

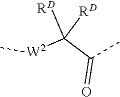

—O—C(R^{D})2-C(O)—. In another preferred example,

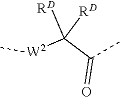

### —O—C(CH3)2-C(O)—.

In another preferred example, R^{E} is selected from the following groups: H, methyl, and ethyl.

In another preferred case, R^{E} is H.

In another preferred example, R^{F}R^{F} is independently selected from the following groups: H, methyl and ethyl.

In another preferred example, R^{F}R^{F} is both H.

In another preferred example,

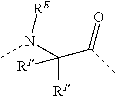

### it is —NH—CH2-C(O)—.

In another preferred example, R⁶ is optionally replaced by a group selected from the lower group: —OR² or —NR⁴R⁵. In another preferred example, R⁶ is —NR⁴R⁵.

In another preferred example, R² is selected from the following group: H, optionally substituted C₁₋₆ alkyl groups.

In another preferred example, —NR⁴R⁵ is a 4 to 10-membered heteronaphthenic alkyl group that is replaced by one or more (e.g., 1, 2, or 3) R³s.

In another preferred example, —NR⁴R⁵ is a 5- to 6-membered heterocyclic alkyl group that is replaced by one or more (e.g., 1, 2, or 3) R³s.

In another preferred example, —NR⁴R⁵ is a 5- to 6-membered heterocyclic alkyl group replaced by 1 R³.

In another preferred example, —NR⁴R⁵ is

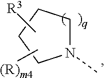

where q is 0, 1, 2, or 3.

In another preferred example, the 5 to 6-member heterocycloalkyl group contains only one N heteroatom as a ring atom.

In another preferred case, —NR⁴R⁵ is replaced by one or more R³s

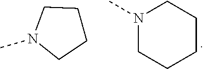

In another preferred example, —NR⁴R⁵ is selected from the following group:

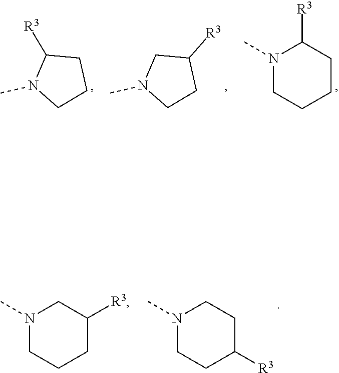

In another preferred example, —NR⁴R⁵ is

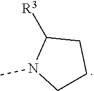

In another preferred example, —NR⁴R⁵ is selected from the following group:

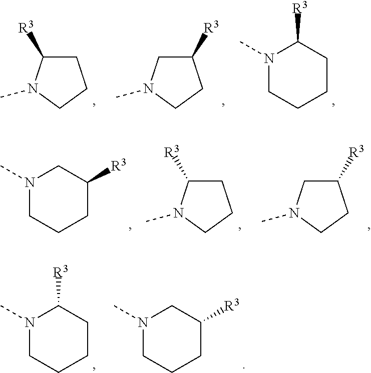

In another preferred example, —NR⁴R⁵ is

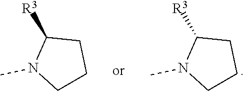

In another preferred example, R³¹ is selected from the following group: H, optionally substituted C₁₋₄ alkyl groups. In another preferred example, R³¹ is selected from the following groups: H, C₁₋₄ alkyls. In another preferred example, R³¹ is selected from the following group: H, methyl group.

In another preferred example, R³² is selected from the following groups: H, C₁₋₄ alkyls. In another preferred example, R³² is selected from the following group: H, methyl group.

In another preferred example, R³³ is selected from the following groups: H, optionally substituted C₁₋₄ alkyl group, —C₁₋₄ alkyl —R³⁴; In another preferred example, R³ is selected from the following groups: —OH, —CH₂OH, —NH2, —N(CH3)₂, —CH2-NH2, —CH2-N(CH3)₂, —CH2-CH2-NH2, —CH2-CH2-N(CH3)₂.

In another preferred example, R³ is —CH2-NH—CH2-R³⁴.

In another preferred example, R³⁴ is selected from the following groups: C₆₋₁₀ aryl groups, 5 to 10 meta heteroaryl groups.

In another preferred example, R³⁴ is selected from the following groups: phenyl, 5- or 6-membered heteroaryl.

In another preferred case, in R³⁴, the C₆₋₁₀ aryl group is replaced by at least one —NH2.

In another preferred example, in R³⁴, the 5 to 10-membered heteroaryl (preferably, 5 or 6-membered heteroaryl) is a heteroaryl group containing 1 or 2 nitrogen heteroatoms as ring atoms; Ideally, a 5 to 10-membered heteroaryl group (preferably, a 5- or 6-membered heteroaryl) comprises one or two nitrogen heteroatoms as ring atoms, and the remaining ring atoms in the heteroaryl group are carbon atoms.

In another preferred example, R³⁴ is selected from the following group:

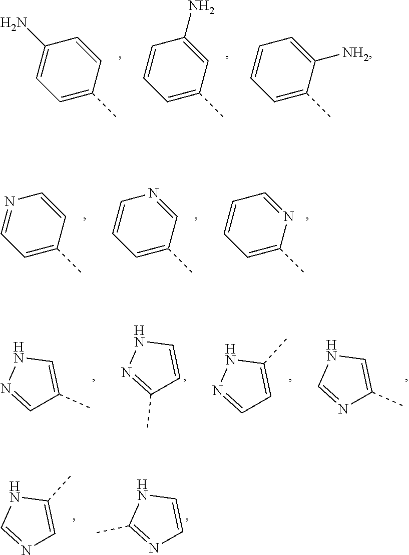

In another preferred example, R⁶ is —NR⁴R⁵.

In another preferred example, R³ is —C₁₋₄ alkylo-N(R³¹)R³²; Better, it is -methylene-N(R³¹)R³².

In another preferred example, W⁴ is —N(R^{s1})—; W⁵ is selected from the following groups: —C(O)—, —S(O)—, —S(O)₂—.

In another preferred case, R¹0 is an unsubstituted or substituted by one or more R^{H}s selected from the following groups: C₁₋₆ alkyls, C₃₋₁₀ naphthenyls, 4 to 10 heterocyclic alkyl groups, C₃₋₁₀ cyclene groups, 4 to 10 members heterocyclic alkyl groups, C₆₋₁₀ aryl groups, and 5 to 10 members heteroaryl groups.

In another preferred example, R¹0 is unsubstituted or replaced by one or more R^{H}s selected from the following groups: C₆₋₁₀ aryl, and 5 to 10 heteroaryl groups.

In another preferred case, R¹0 is a phenyl group that is replaced by one or more R^{H}s.

In another preferred example, R¹0 is a phenyl group replaced by a 4-R^{H} (R^{H} at position 4).

In another preferred example, the compound is selected from Tables A-I

TABLE A-I

| Numerator | |
|---|---|
| number | structural formula |
| 115-010 | 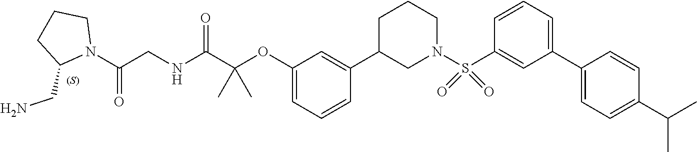 |
| 115-011 | 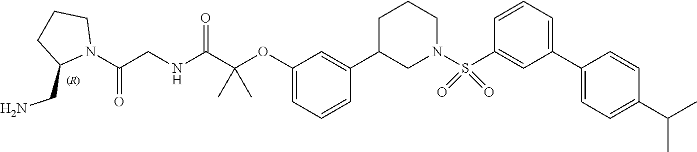 |
| 115-012 | 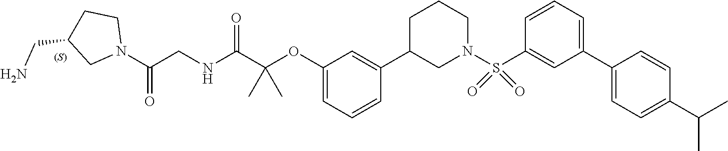 |
| 115-013 | 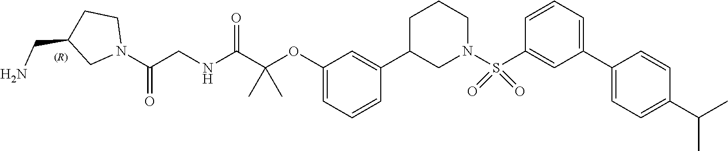 |
| 115-014 | 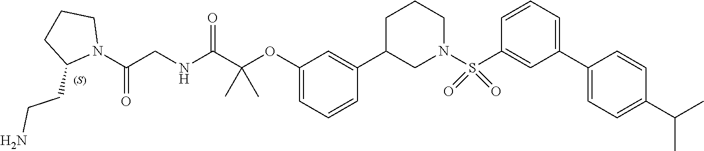 |
| 115-015 | 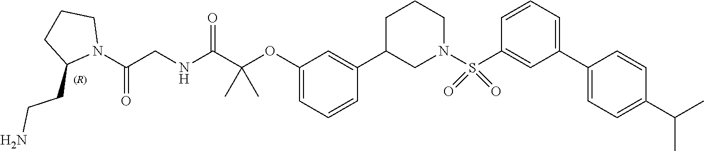 |
| 115-016 | 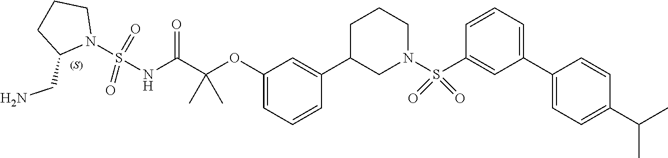 |
| 115-017 | 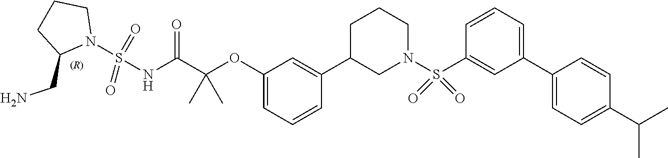 |
| 115-018 | 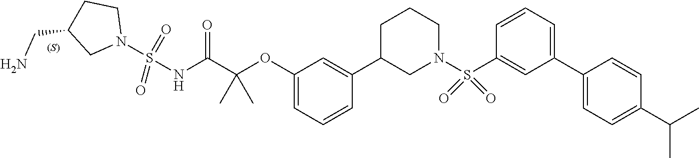 |
| 115-019 | 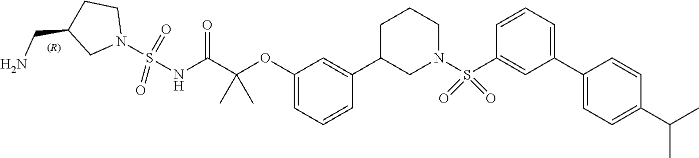 |
| 115-020 | 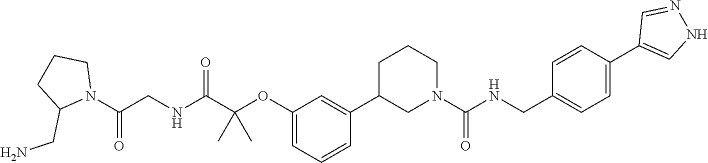 [SMILES: CC(C)(Oc1cccc(C2CCCN(C(=O)NCc3ccc(-c4cn[nH]c4)cc3)C2)c1)C(=O)NCC(=O)N1CCCC1CN] |
| 115-021 | 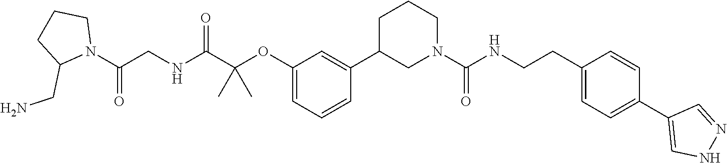 [SMILES: CC(C)(Oc1cccc(C2CCCN(C(=O)NCCc3ccc(-c4cn[nH]c4)cc3)C2)c1)C(=O)NCC(=O)N1CCCC1CN] |
| 115-022 | 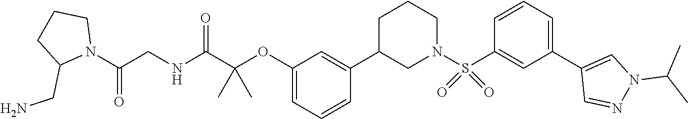 [SMILES: CC(C)n1cc(-c2cccc(S(=O)(=O)N3CCCC(c4cccc(OC(C)(C)C(=O)NCC(=O)N5CCCC5CN)c4)C3)c2)cn1] |
| 115-023 | 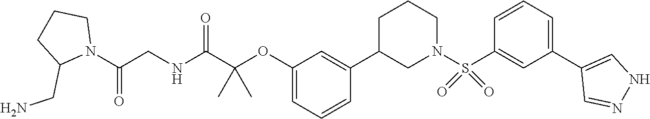 [SMILES: CC(C)(Oc1cccc(C2CCCN(S(=O)(=O)c3cccc(-c4cn[nH]c4)c3)C2)c1)C(=O)NCC(=O)N1CCCC1CN] |
| 115-024 | 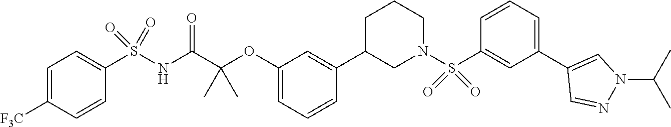 [SMILES: CC(C)n1cc(-c2cccc(S(=O)(=O)N3CCCC(c4cccc(OC(C)(C)C(=O)NS(=O)(=O)c5ccc(C(F)(F)F)cc5)c4)C3)c2)cn1] |
| 115-025 | 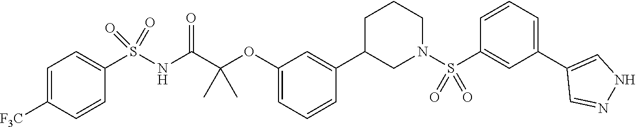 [SMILES: CC(C)(Oc1cccc(C2CCCN(S(=O)(=O)c3cccc(-c4cn[nH]c4)c3)C2)c1)C(=O)NS(=O)(=O)c1ccc(C(F)(F)F)cc1] |
| 115-026 | 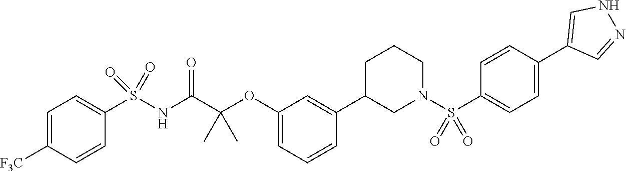 [SMILES: CC(C)(Oc1cccc(C2CCCN(S(=O)(=O)c3ccc(-c4cn[nH]c4)cc3)C2)c1)C(=O)NS(=O)(=O)c1ccc(C(F)(F)F)cc1] |
| 115-027 | 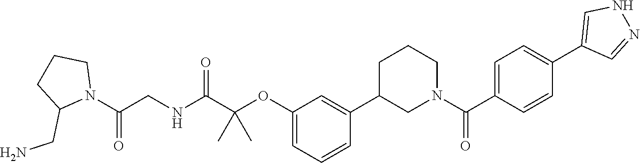 [SMILES: CC(C)(Oc1cccc(C2CCCN(C(=O)c3ccc(-c4cn[nH]c4)cc3)C2)c1)C(=O)NCC(=O)N1CCCC1CN] |
| 115-028 | 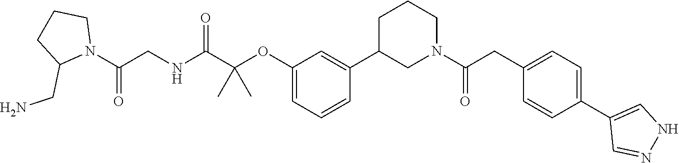 [SMILES: CC(C)(Oc1cccc(C2CCCN(C(=O)Cc3ccc(-c4cn[nH]c4)cc3)C2)c1)C(=O)NCC(=O)N1CCCC1CN] |
| 115-029 | 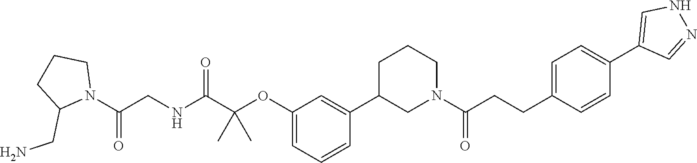 [SMILES: CC(C)(Oc1cccc(C2CCCN(C(=O)CCc3ccc(-c4cn[nH]c4)cc3)C2)c1)C(=O)NCC(=O)N1CCCC1CN] |
| 115-030 | 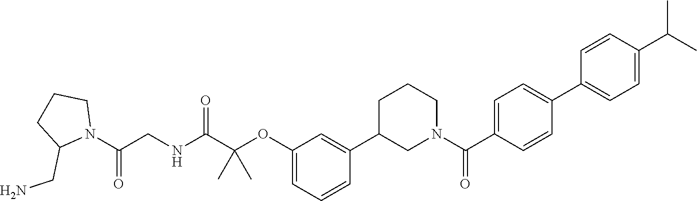 [SMILES: CC(C)c1ccc(-c2ccc(C(=O)N3CCCC(c4cccc(OC(C)(C)C(=O)NCC(=O)N5CCCC5CN)c4)C3)cc2)cc1] |
| 115-031 | 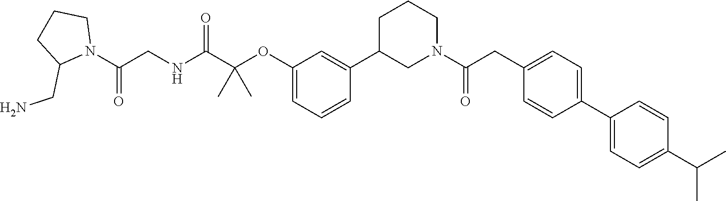 [SMILES: CC(C)c1ccc(-c2ccc(CC(=O)N3CCCC(c4cccc(OC(C)(C)C(=O)NCC(=O)N5CCCC5CN)c4)C3)cc2)cc1] |
| 115-032 | 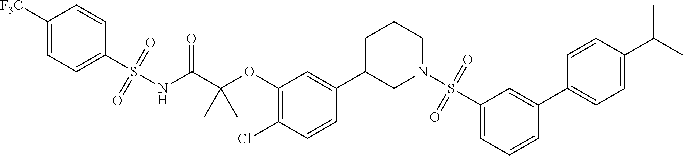 [SMILES: CC(C)c1ccc(-c2cccc(S(=O)(=O)N3CCCC(c4ccc(Cl)c(OC(C)(C)C(=O)NS(=O)(=O)c5ccc(C(F)(F)F)cc5)c4)C3)c2)cc1] |
| 115-033 | 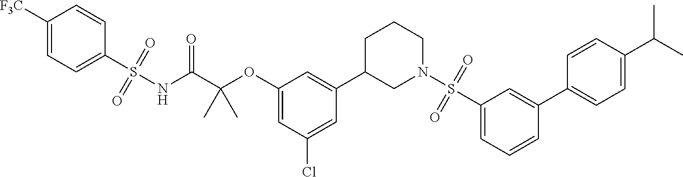 [SMILES: CC(C)c1ccc(-c2cccc(S(=O)(=O)N3CCCC(c4cc(Cl)cc(OC(C)(C)C(=O)NS(=O)(=O)c5ccc(C(F)(F)F)cc5)c4)C3)c2)cc1] |
| 115-034 | 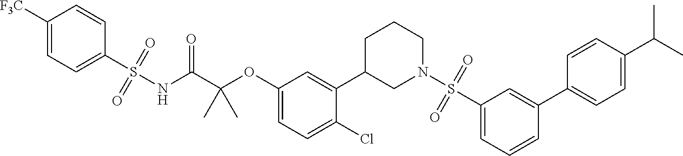 [SMILES: CC(C)c1ccc(-c2cccc(S(=O)(=O)N3CCCC(c4cc(OC(C)(C)C(=O)NS(=O)(=O)c5ccc(C(F)(F)F)cc5)ccc4Cl)C3)c2)cc1] |
| 115-035 | 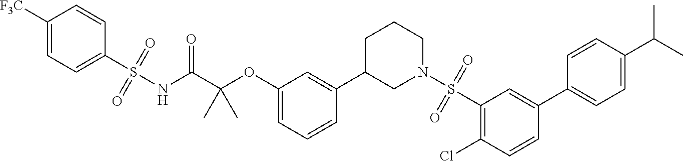 [SMILES: CC(C)c1ccc(-c2ccc(Cl)c(S(=O)(=O)N3CCCC(c4cccc(OC(C)(C)C(=O)NS(=O)(=O)c5ccc(C(F)(F)F)cc5)c4)C3)c2)cc1] |
| 115-036 | 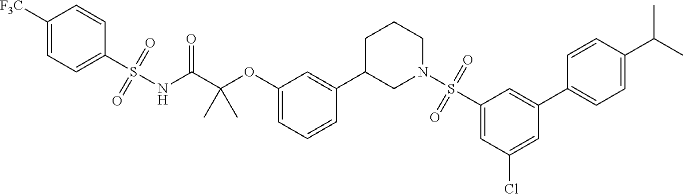 [SMILES: CC(C)c1ccc(-c2cc(Cl)cc(S(=O)(=O)N3CCCC(c4cccc(OC(C)(C)C(=O)NS(=O)(=O)c5ccc(C(F)(F)F)cc5)c4)C3)c2)cc1] |
| 115-037 | 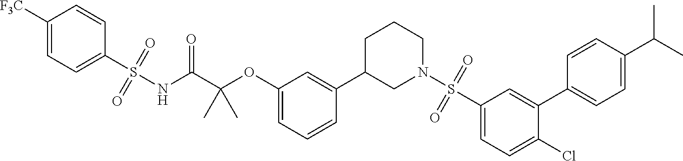 [SMILES: CC(C)c1ccc(-c2cc(S(=O)(=O)N3CCCC(c4cccc(OC(C)(C)C(=O)NS(=O)(=O)c5ccc(C(F)(F)F)cc5)c4)C3)ccc2Cl)cc1] |
| 115-038 | 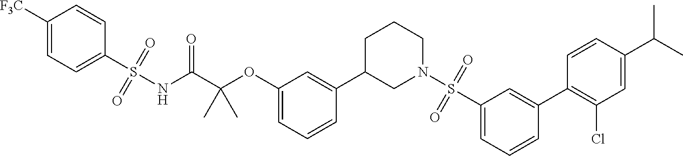 [SMILES: CC(C)c1ccc(-c2cccc(S(=O)(=O)N3CCCC(c4cccc(OC(C)(C)C(=O)NS(=O)(=O)c5ccc(C(F)(F)F)cc5)c4)C3)c2)c(Cl)c1] |
| 115-039 | 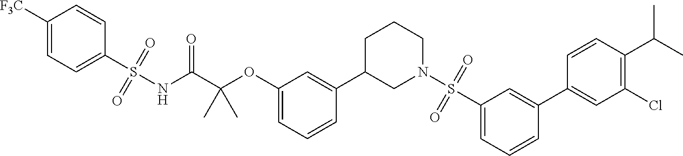 [SMILES: CC(C)c1ccc(-c2cccc(S(=O)(=O)N3CCCC(c4cccc(OC(C)(C)C(=O)NS(=O)(=O)c5ccc(C(F)(F)F)cc5)c4)C3)c2)cc1Cl] |

In another preferred example, the compound is selected from Tables B-I

TABLE B-I

| Numerator | |
|---|---|
| number | structural formula |
| 115-040 | 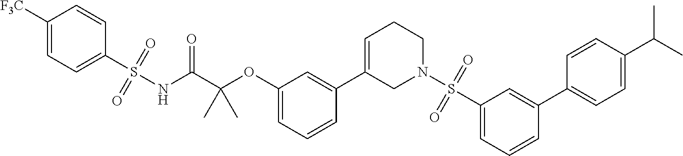 [SMILES: CC(C)c1ccc(-c2cccc(S(=O)(=O)N3CCC=C(c4cccc(OC(C)(C)C(=O)NS(=O)(=O)c5ccc(C(F)(F)F)cc5)c4)C3)c2)cc1] |
| 115-041 | 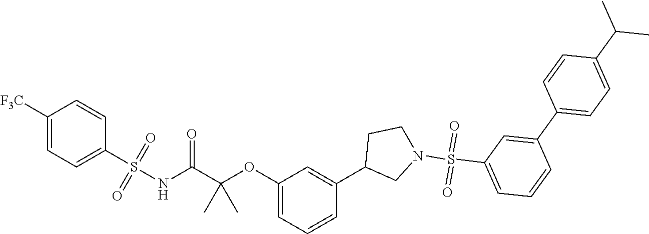 [SMILES: CC(C)c1ccc(-c2cccc(S(=O)(=O)N3CCC(c4cccc(OC(C)(C)C(=O)NS(=O)(=O)c5ccc(C(F)(F)F)cc5)c4)C3)c2)cc1] |
| 115-042 | 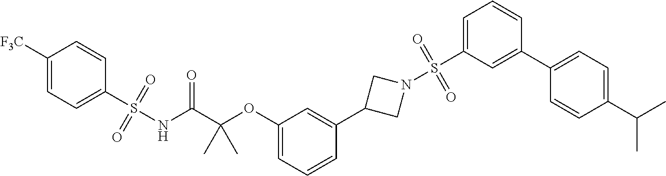 [SMILES: CC(C)c1ccc(-c2cccc(S(=O)(=O)N3CC(c4cccc(OC(C)(C)C(=O)NS(=O)(=O)c5ccc(C(F)(F)F)cc5)c4)C3)c2)cc1] |
| 115-043 | 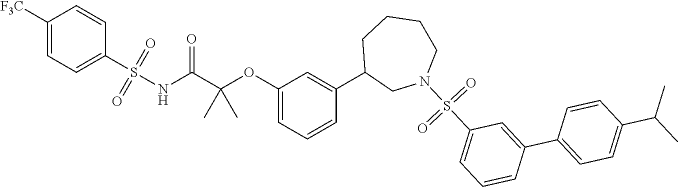 [SMILES: CC(C)c1ccc(-c2cccc(S(=O)(=O)N3CCCCC(c4cccc(OC(C)(C)C(=O)NS(=O)(=O)c5ccc(C(F)(F)F)cc5)c4)C3)c2)cc1] |
| 115-044 | 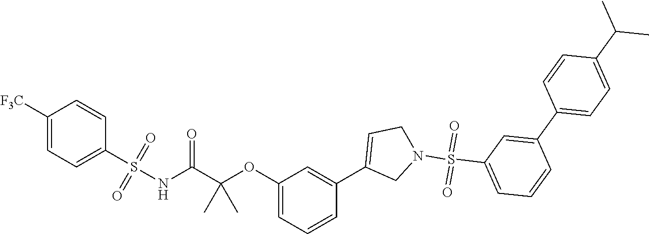 [SMILES: CC(C)c1ccc(-c2cccc(S(=O)(=O)N3CC=C(c4cccc(OC(C)(C)C(=O)NS(=O)(=O)c5ccc(C(F)(F)F)cc5)c4)C3)c2)cc1] |
| 115-045 | 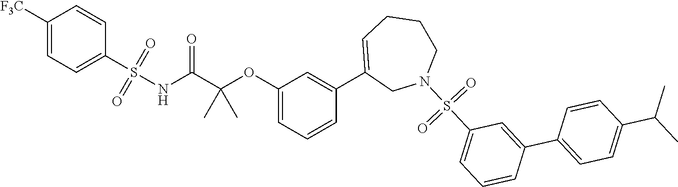 [SMILES: CC(C)c1ccc(-c2cccc(S(=O)(=O)N3CCCC=C(c4cccc(OC(C)(C)C(=O)NS(=O)(=O)c5ccc(C(F)(F)F)cc5)c4)C3)c2)cc1] |
| 115-046 | 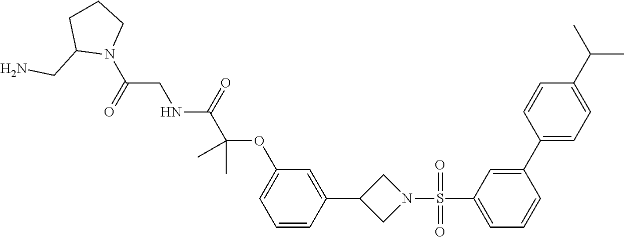 [SMILES: CC(C)c1ccc(-c2cccc(S(=O)(=O)N3CC(c4cccc(OC(C)(C)C(=O)NCC(=O)N5CCCC5CN)c4)C3)c2)cc1] |
| 115-047 | 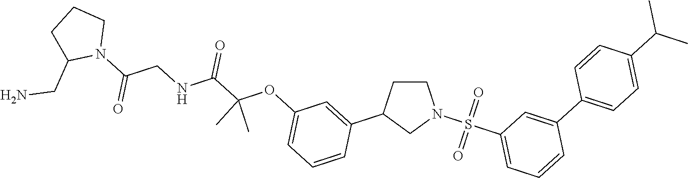 [SMILES: CC(C)c1ccc(-c2cccc(S(=O)(=O)N3CCC(c4cccc(OC(C)(C)C(=O)NCC(=O)N5CCCC5CN)c4)C3)c2)cc1] |
| 115-048 | 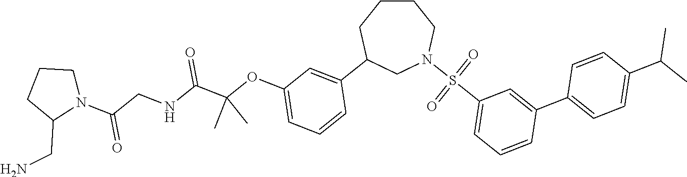 [SMILES: CC(C)c1ccc(-c2cccc(S(=O)(=O)N3CCCCC(c4cccc(OC(C)(C)C(=O)NCC(=O)N5CCCC5CN)c4)C3)c2)cc1] |
| 115-049 | 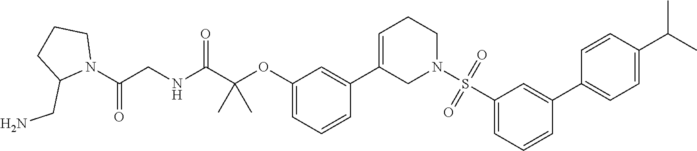 [SMILES: CC(C)c1ccc(-c2cccc(S(=O)(=O)N3CCC=C(c4cccc(OC(C)(C)C(=O)NCC(=O)N5CCCC5CN)c4)C3)c2)cc1] |
| 115-050 | 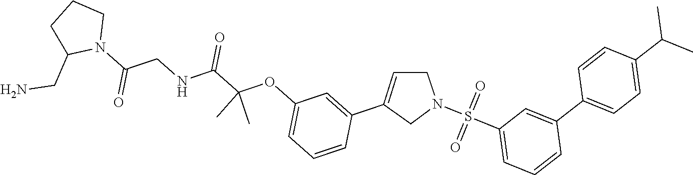 [SMILES: CC(C)c1ccc(-c2cccc(S(=O)(=O)N3CC=C(c4cccc(OC(C)(C)C(=O)NCC(=O)N5CCCC5CN)c4)C3)c2)cc1] |
| 115-051 | 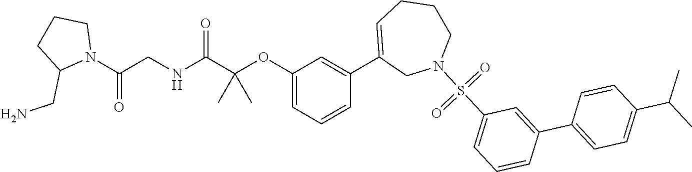 [SMILES: CC(C)c1ccc(-c2cccc(S(=O)(=O)N3CCCC=C(c4cccc(OC(C)(C)C(=O)NCC(=O)N5CCCC5CN)c4)C3)c2)cc1] |

In another preferred example, the compound is selected from Tables C-I

TABLE C-I

| Numerator | |
|---|---|
| number | structural formula |
| 115-052 | 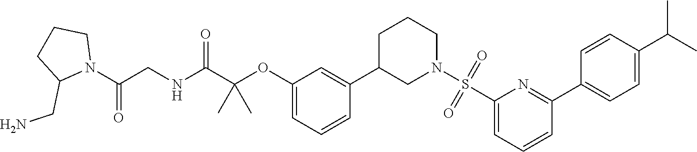 [SMILES: CC(C)c1ccc(-c2cccc(S(=O)(=O)N3CCCC(c4cccc(OC(C)(C)C(=O)NCC(=O)N5CCCC5CN)c4)C3)n2)cc1] |
| 115-053 | 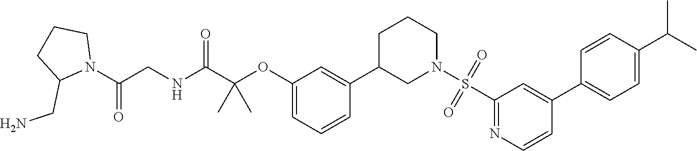 [SMILES: CC(C)c1ccc(-c2ccnc(S(=O)(=O)N3CCCC(c4cccc(OC(C)(C)C(=O)NCC(=O)N5CCCC5CN)c4)C3)c2)cc1] |
| 115-054 | 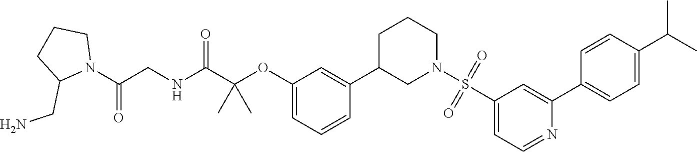 [SMILES: CC(C)c1ccc(-c2cc(S(=O)(=O)N3CCCC(c4cccc(OC(C)(C)C(=O)NCC(=O)N5CCCC5CN)c4)C3)ccn2)cc1] |
| 115-055 | 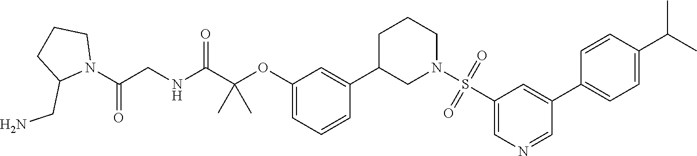 [SMILES: CC(C)c1ccc(-c2cncc(S(=O)(=O)N3CCCC(c4cccc(OC(C)(C)C(=O)NCC(=O)N5CCCC5CN)c4)C3)c2)cc1] |
| 115-056 | 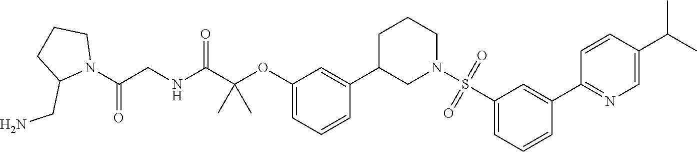 [SMILES: CC(C)c1ccc(-c2cccc(S(=O)(=O)N3CCCC(c4cccc(OC(C)(C)C(=O)NCC(=O)N5CCCC5CN)c4)C3)c2)nc1] |
| 115-057 | 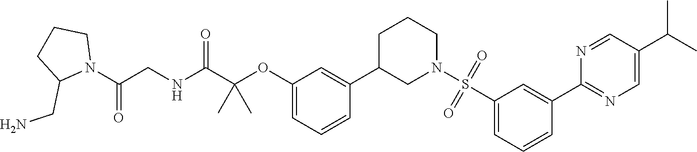 [SMILES: CC(C)c1cnc(-c2cccc(S(=O)(=O)N3CCCC(c4cccc(OC(C)(C)C(=O)NCC(=O)N5CCCC5CN)c4)C3)c2)nc1] |
| 115-058 | 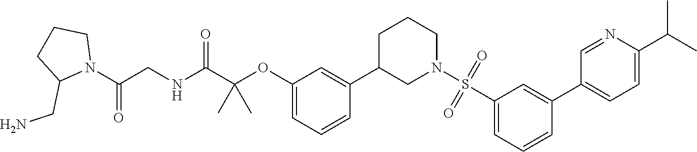 [SMILES: CC(C)c1ccc(-c2cccc(S(=O)(=O)N3CCCC(c4cccc(OC(C)(C)C(=O)NCC(=O)N5CCCC5CN)c4)C3)c2)cn1] |
| 115-059 | 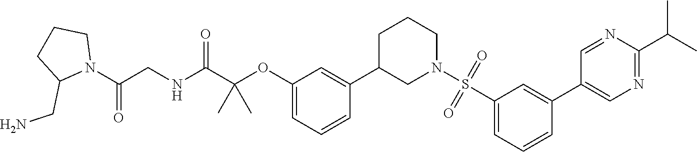 [SMILES: CC(C)c1ncc(-c2cccc(S(=O)(=O)N3CCCC(c4cccc(OC(C)(C)C(=O)NCC(=O)N5CCCC5CN)c4)C3)c2)cn1] |
| 115-060 | 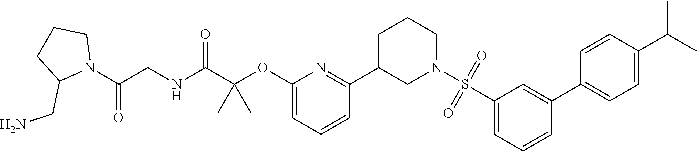 [SMILES: CC(C)c1ccc(-c2cccc(S(=O)(=O)N3CCCC(c4cccc(OC(C)(C)C(=O)NCC(=O)N5CCCC5CN)n4)C3)c2)cc1] |
| 115-061 | 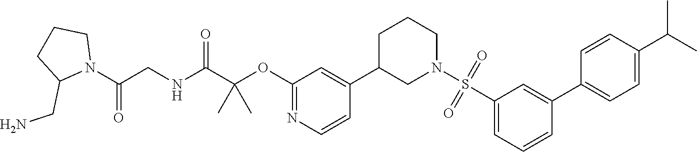 [SMILES: CC(C)c1ccc(-c2cccc(S(=O)(=O)N3CCCC(c4ccnc(OC(C)(C)C(=O)NCC(=O)N5CCCC5CN)c4)C3)c2)cc1] |
| 115-062 | 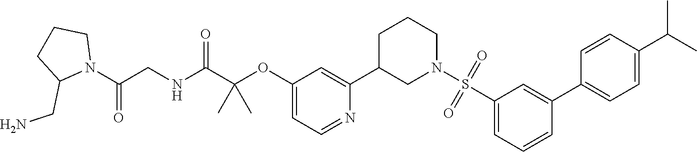 [SMILES: CC(C)c1ccc(-c2cccc(S(=O)(=O)N3CCCC(c4cc(OC(C)(C)C(=O)NCC(=O)N5CCCC5CN)ccn4)C3)c2)cc1] |
| 115-063 | 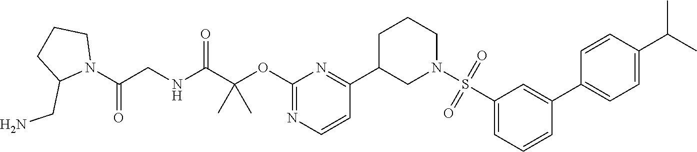 [SMILES: CC(C)c1ccc(-c2cccc(S(=O)(=O)N3CCCC(c4ccnc(OC(C)(C)C(=O)NCC(=O)N5CCCC5CN)n4)C3)c2)cc1] |
| 115-064 | 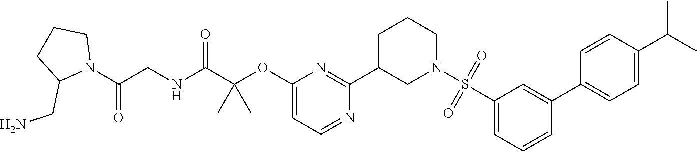 [SMILES: CC(C)c1ccc(-c2cccc(S(=O)(=O)N3CCCC(c4nccc(OC(C)(C)C(=O)NCC(=O)N5CCCC5CN)n4)C3)c2)cc1] |
| 115-065 | 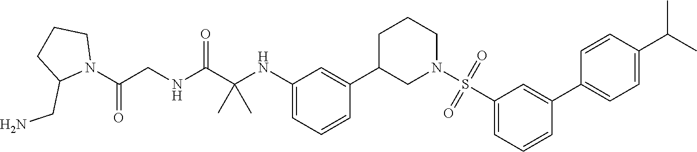 [SMILES: CC(C)c1ccc(-c2cccc(S(=O)(=O)N3CCCC(c4cccc(NC(C)(C)C(=O)NCC(=O)N5CCCC5CN)c4)C3)c2)cc1] |
| 115-066 | 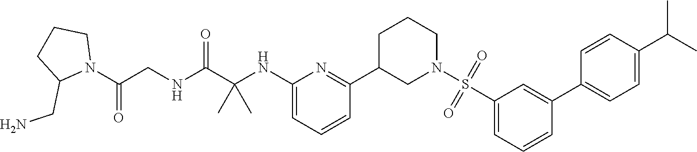 [SMILES: CC(C)c1ccc(-c2cccc(S(=O)(=O)N3CCCC(c4cccc(NC(C)(C)C(=O)NCC(=O)N5CCCC5CN)n4)C3)c2)cc1] |
| 115-067 | 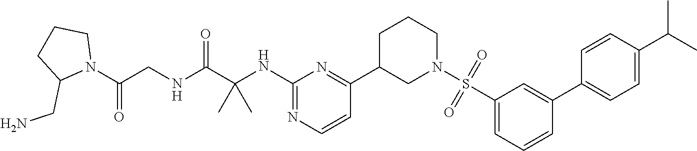 [SMILES: CC(C)c1ccc(-c2cccc(S(=O)(=O)N3CCCC(c4ccnc(NC(C)(C)C(=O)NCC(=O)N5CCCC5CN)n4)C3)c2)cc1] |
| 115-068 | 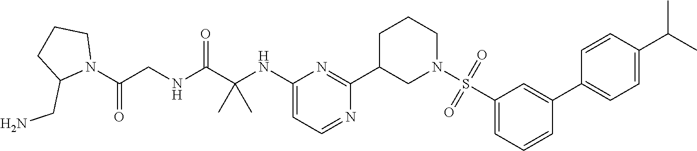 [SMILES: CC(C)c1ccc(-c2cccc(S(=O)(=O)N3CCCC(c4nccc(NC(C)(C)C(=O)NCC(=O)N5CCCC5CN)n4)C3)c2)cc1] |
| 115-069 | 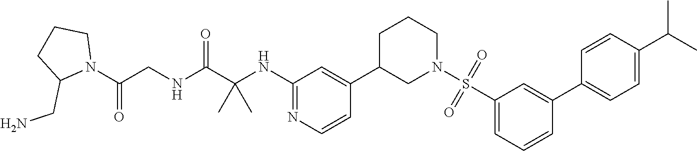 [SMILES: CC(C)c1ccc(-c2cccc(S(=O)(=O)N3CCCC(c4ccnc(NC(C)(C)C(=O)NCC(=O)N5CCCC5CN)c4)C3)c2)cc1] |

In another preferred example, ring A, ring B, ring C, L¹, W², R^{A}, R^{B}, R^{C}, R^{D}, R⁷, R9, subscript m1, subscript m2, and subscript m3 are each independently the embodiment compound or the corresponding group in the specific compound in Tables A-I, B-I, and C-I.

In another preferred example, ring A, ring B, ring C, L¹, W², R^{A}, R^{B}, R^{C}, R^{D}, R^{E}, R^{F}R^{F}, R⁷, R⁶, subscript m1, subscript m2, and subscript m3 are each independently the embodiment compound or the corresponding group in the specific compound in Tables A-I, B-I, and C-I.

In another preferred example, ring A, ring B, ring C, L¹, W², R^{A}, R^{B}, RC, R^{D}, R⁷, R¹0, W⁵, W⁴, subscript m1, subscript m2, and subscript m3 are each independently the embodiment compound or the corresponding group in the specific compound in Tables A-I, B-I, and C-I.

In another preferred example, ring A, ring B, ring C, L¹, W², R^{A}, R^{B}, RC, R^{D}, R⁷, R^{E}, R^{F}R^{F}, R⁶, subscript m1, subscript m2 and subscript m3 can also be defined as in the second aspect.

In the second aspect of the present invention, a compound or a pharmaceutically acceptable salt thereof, or its isomer, solvent complex, crystal form, or prodrug is provided, and the compound is shown in Formula II

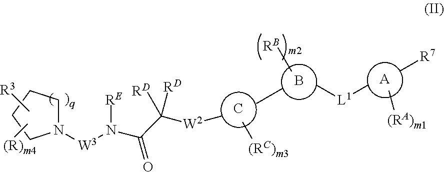

thereinto
q is 0, 1, 2 or 3;
R³ is selected from the following groups: H, —OR³¹, —C₁₋₄ alky-OR³¹, —N(R³²)R³³, —C₁₋₄ alky-N(R³¹)R³²;
m4 is 0, 1, 2, 3, 4, 5, 6 or 7;
R³¹ is selected from the following groups: H, optionally substituted C₁₋₄ alkyl groups, R³⁴, —C₁₋₄ alkyl —R³⁴;
R³² is selected from the following group: H, optionally substituted C₁₋₄ alkyl groups;
R³³ is selected from the following groups: H, optionally substituted C₁₋₄ alkyl groups, R³⁴, —C₁₋₄ alkyl —R³⁴;
R³⁴ is selected from the following groups: C₃₋₁₀ naphthenyl, 4 to 8 members heterocyclic alkyl groups, C₆₋₁₀ aryl groups, 5 to 10 membered heteroaryl groups, C₃₋₁₀ cyclene groups, 4 to 10 membered heterocyclic alkyl groups; wherein, the naphthenic alkyl group, the aryl group, the heteroaryl group, the cyclic alkene group and the heterocyclic alkyl group are optionally replaced by one or more groups selected from the following group: —NH2, R;
W3 is selected from the following groups: —C(O)—, —S(O)—, —S(O)₂—, —C(R^{F}R^{F})₂—C(O)—, —C(R^{F}R^{F}) ₂₋ₛ(O)—, —C(R^{F}R^{F})₂—S(O)₂— (Preferably, —C(R^{F}R^{F})₂ in W3- connected with N(R^{E}));
R^{F}R^{F} is independently selected from the following groups: H, optionally substituted C₁₋₄ alkyl groups, optionally substituted C₃₋₁₀ naphthenic alkyl groups, optionally substituted 4 to 10 membered heteronaphthenic alkyl groups; or two R^{F}R^{F}s and carbon atoms connected to them together form an optionally substituted C₃₋₆ naphthenic group or an optionally substituted 4 to 6-membered heterocyclic group;
R^{E} is selected from the following groups: H, optionally substituted C₁₋₄ alkyl groups, optionally substituted C₃₋₁₀ naphthenic alkyl groups, optionally substituted 4 to 10 membered heteronaphthenic alkyl groups;
R^{D} is independently selected from the following groups: H, optionally substituted C₁₋₄ alkyl groups; Alternatively, the two R^{D}s and the carbon atoms attached to them together form groups selected from the following groups: optionally substituted C₃₋₁₀ naphthenic alkyl groups, optionally substituted 4 to 10-membered heteronaphthenic alkyl groups;
W² is selected from the following group: —O—, —S—, —N(R^{s1})—;
Ring C is optionally replaced by the following groups: C₆₋₁₀ aryl groups, and 5 to 10 membered heteroaryl groups;
m3=0, 1, 2, 3 or 4;
Each R^{C} is R^{C1} or R^{s1} independently;
Each R^{C1} is independently selected from the following groups: halogens, optionally substituted C₁₋₆ alkyl groups, optionally substituted C₁₋₆ halogenated alkyl groups, hydroxyl groups and optionally substituted C₁₋₆ alkoxy groups, and optionally substituted C₁₋₆ halogenated alkoxy groups;
Ring B is the ring selected from the following group of alternatives: C₃₋₁₂ naphthenic alkyl group, 4 to 12 metaphthenic alkyl groups, C₃₋₁₀ cyclene groups, 4 to 10 membered heteronaphthenic alkyl groups;
Each R^{B} is R^{B1}, R^{s1} or R^{s2} independently;
Each R^{B1} is independently selected from the following groups: halogens, hydroxyl groups, cyanos, optionally substituted C₁₋₆ alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₃₋₁₀ cyclic alkyl groups, optionally substituted 4 to 10-membered heterocyclic alkyl groups, optionally substituted C₆₋₁₀ aryl groups, and optionally substituted 5 to 10-membered heteroaryl groups;
m2=0, 1, 2, 3 or 4;
L¹ is —X—(W¹)ₙ₁—;
X is selected from the following groups: —C(O)—, —S(O)— and —S(O)₂—;
Each W¹ is independently selected from the following groups: none, —O—, —S—, —C(O)—, —S(O)—, —S(O)₂—, —N(R^{s1})—, —C(R^{s2})₂—;
subscript n1=0, 1, 2 or 3;
Ring A is optionally replaced by the following group: C₆₋₁₀ aryl; 5 to 10 membered heteroaryl groups; C₆₋₁₀ aryl groups substituted by C₃₋₁₀ naphthenic alkyl groups, 4 to 10 meta heterocyclic alkyl groups, C₃₋₁₀ cyclene groups, 4 to 10 membered heterocyclic alkyl groups, C₆₋₁₀ aryl groups or 5 to 10 membered heteroaryl groups; 5 to 10 membered heteroaryl groups replaced by C₃₋₁₀ naphthenic alkyl groups, 4 to 10 metaphnocyclic alkyl groups, C₃₋₁₀ cyclene groups, 4 to 10 membered heterocyclic alkyl groups, C₆₋₁₀ aryl groups or 5 to 10 membered heteroaryl groups; C₆₋₁₀ aryl fused with C₃₋₁₀ naphthenyl, 4 to 10 heteronaphthenic alkyl groups, C₃₋₁₀ cyclene groups, 4 to 10 members heterocyclic alkenyl groups, C₆₋₁₀ aryl groups or 5 to 10 member heteroaryl groups; 5 to 10 membered heteroaryl groups fused with C₃₋₁₀ naphthenyl, 4 to 10 metaphthenic alkyl groups, C₃₋₁₀ cyclene groups, 4 to 10 membered heterocyclic alkyl groups, C₆₋₁₀ aryl groups, or 5 to 10 membered heteroaryl groups;
m1=0, 1, 2, 3 or 4;
Each R^{A} is R^{A}R^{A1}, R^{s1} or R^{s2} independently;
Each R^{A}R^{A1} is independently selected from the following groups: halogens, optionally substituted C₁₋₆ alkyl groups, optionally substituted C₁₋₆ halogenated alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₆₋₁₀ aryl groups, optionally replaced 5 to 10 membered heteroaryl groups; Alternatively, when two R^{A}R^{A1}s are located on adjacent ring atoms, the two R^{A}R^{A1}s and their adjacent ring atoms together form a selected ring from the following group: optionally substituted C₃₋₁₀ naphthenic alkyl group, optionally substituted 4 to 10-membered heterocyclic alkyl group, optionally substituted C₃₋₁₀ cyclic alkyl group, optionally substituted 4 to 10-membered heterocyclic alkene group, optionally substituted C₆₋₁₀ aryl group, optionally substituted C₆₋₁₀ aryl group, optionally replace 5 to 10 membered heteroaryl groups;
R⁷ is optionally substituted groups from the following groups: none, C₁₋₆ alkyl groups, C₃₋₁₀ naphthenyl groups, 4 to 10 members heterocyclic alkyl groups, C₃₋₁₀ cyclic alkyl groups, 4 to 10 members heterocyclic alkyl groups, C₆₋₁₀ aryl groups, and 5 to 10 member heteroaryl groups.

R^{s1} is independently selected from the following groups: H, optionally substituted C₁₋₄ alkyl group, optionally substituted C₃₋₆ naphthenic group, and optionally substituted 4 to 6-membered heterocyclic group;
R^{s2} are independently selected from the following groups: H, optionally substituted C₁₋₄ alkyl groups, optionally substituted C₃₋₆ naphthenic alkyl groups, and optionally substituted 4 to 6-membered heterocyclic groups; or two R^{s2} and the carbon atoms attached to them together form an optionally substituted C₃₋₆ naphthenic group or an optionally substituted 4 to 6-membered heterocyclic group;
Unless otherwise defined, said optional substitution means an unsubstituted or group of one or more (e.g., 1, 2, 3, or 4) hydrogen substituted by an R substituent;
R are independently selected from the following groups: D, halogens, C₁₋₆ alkyls, C₁₋₆ halogenated alkyls, C₁₋₆ hydroxyalkyls, C₂₋₆ alkyl groups, C₂₋₆ alkyl groups, —CN, —OR′, —NO2, —NR′R″, —SR′, OC(O)R′, —C(O)R′, —CO2R′, —CONR′, —OC(O)NR′R″, —NR″C(O)R′, —NR″—C(O)NR′R″, —NR′C (O)₂R, —S(O)R′, —S(O)₂R′, —S(O)₂NR′R″, NR″S(O)₂R′, any C₃₋₁₀ naphthenic alkyl group replaced by one or more R′, any 4 to 10 member heterocycloalkyl group replaced by one or more R′, any C₆₋₁₀ aryl group replaced by one or more R′, any 5 to 10 member heteroaryl group replaced by one or more R′, any —C₁₋₄ alkyl group C₃₋₁₀ naphthenic group replaced by one or more R″, any —C₁₋₄ alkyl group replaced by one or more R″, choose —C₁₋₄ alkyl-C₆₋₁₀ aryl group replaced by one or more R″″, choose —C₁₋₄ alkyl-5 to 10 aryl group replaced by one or more R″s;
Each R′ is independently selected from the following groups: H, D, C₁₋₆ alkyls, C₁₋₆ halogenated alkyls, any C₃₋₁₀ naphthenic alkyl group replaced by one or more R′″, any 4 to 10 heterocyclic alkyl group replaced by one or more R′″, any C₆₋₁₀ aryl group replaced by one or more R′″, any 5 to 10 membered heteroaryl groups replaced by one or more R″s, any —C₁₋₄ alkyl group replaced by one or more R″—C₃₋₁₀ naphthenic alkyl groups, choose —C₁₋₄ alkyl-4 to 10-membered heterocyclic alkyl groups replaced by one or more R′″, choose —C₁₋₄ alkyl —C₆₋₁₀ aryl group replaced by one or more R′″, choose —C₁₋₄ alkyl-5 to 10-member heteroaryl group replaced by one or more R′″;
Each R″ is selected from the following groups: H, D, C₁₋₄ alkyls, C₁₋₄ halogenated alkyls, and C₃₋₄ naphthenic groups;
Each R′″ is independently selected from the following groups: D, halogen, hydroxyl group, nitro, CN, C₁₋₆ alkyl group, C₁₋₆ halogenated alkyl group.

In another preferred example, W3 is —C(R^{F}R^{F})₂—C(O)—, and —C(R^{F}R^{F})₂—is connected to N(R^{E}).

In another preferred example, ring B is optionally replaced by the following groups: C₃₋₁₂ naphthenic alkyl groups, 4 to 12-member heterocyclic alkyl groups.

In another preferred example, ring B is

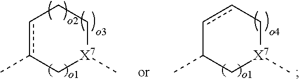

wherein [symbol] it is a single or double bond, X⁷ is N or CH, o1 is 1 or 2, o2 is 0, 1, 2 or 3, o3 is 0 or 1, and o4 is 0, 1, 2 or 3.

In another preferred example, X⁷ is N.

In another preferred example, o1 is 1.

In another preferred case, when X⁷ is N, N in ring B is connected to L¹.

In another preferred example, ring B is

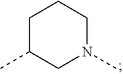

Preferably, N in ring B is connected to L¹.

In another preferred case, R^{B}s are either R^{s1} or R^{s2}.

In another preferred example, m2=0.

In another preferred example,

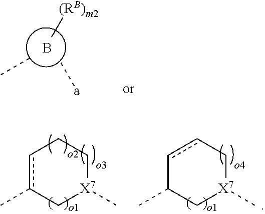

In another preferred example,

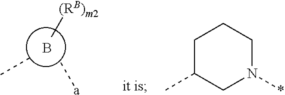

where * refers to the connection to L¹.

In another preferred case, n1=0.

In another preferred case, n1=0, 1, or 2. In another preferred case, each W¹ is independently —C(R^{s2})₂—(preferably, —CH₂—).

In another preferred example, L¹ is —X—; Preferably, L¹ is —CO— or —SO2-. In another preferred example, L¹ is —SO₂—.

In another preferred example, ring C is

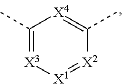

where X¹, X², X³ and X⁴ are independently selected from the following groups: CH and N.

In another preferred example, in ring C, up to 1 or 2 of X¹, X², X³, and X⁴ are N.

In another preferred example, ring C is

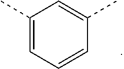

In another preferred example, m3=0. In another preferred example, m3=1, 2, 3, or 4.

In another preferred example, R^{C1} is selected from the following groups: halogens (preferably F, Cl), C₁₋₆ halogenated alkyl groups (preferably trifluoromethyl), and C₁₋₆ alkoxy groups (preferably methoxy).

In another preferred example, ring C is

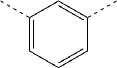

m3=0, 1 or 2; R^{C} is H or R^{C1}; R^{C1} is selected from the following groups: halogens (preferably F, Cl), C₁₋₆ halogenated alkyl groups (preferably trifluoromethyl), and C₁₋₆ alkoxyl groups (preferably methoxy groups); Better, R^{C1} is halogen.

In another preferred example, the compound is shown in Formula II-1

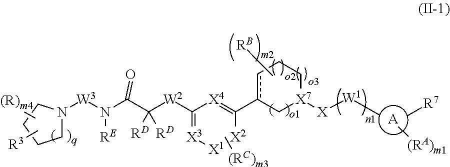

thereinto
[symbol] is single or double bond; X⁷ is N or CH; o1 is 1 or 2, o2 is 0, 1, 2 or 3, o3 is 0 or 1;
X¹, X², X³ and X⁴ are independently selected from the following groups: CH and N.

In another preferred example, the compound is shown in Formula II-2

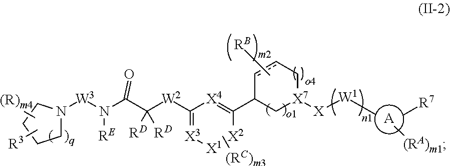

thereinto
[symbol] is single or double bond; X⁷ is N or CH; o1 is 1 or 2, o4 is 0, 1, 2 or 3; X¹, X², X³ and X⁴ are independently selected from the following groups: CH and N.

In another preferred example, the compound is shown in Equations II-3 or II-4

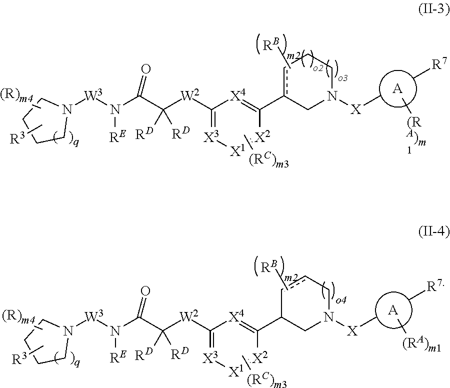

In another preferred example, q is 1 or 2.

In another preferred example,

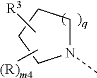

selected from the following group:

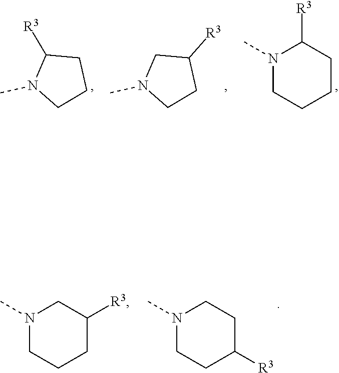

In another preferred example,

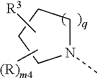

selected from the following group:

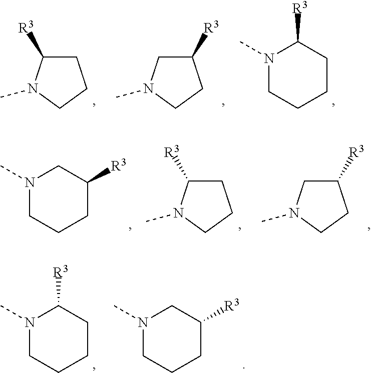

In another preferred example, the compound is shown in Equation A

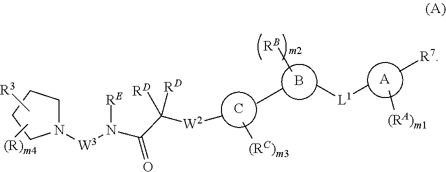

In another preferred example,

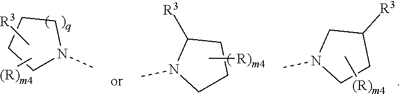

In another preferred example, m4=0.

In another preferred example,

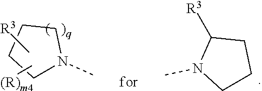

In another preferred example,

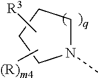

select from the following group:

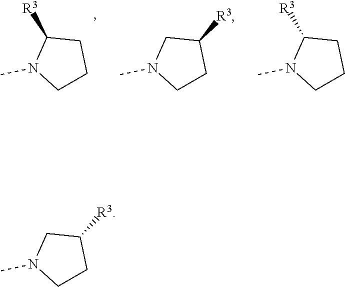

In another preferred example,

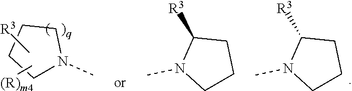

In another preferred example, R is selected from the following group: H, optionally substituted C₁₋₄ alkyl groups. In another preferred example, R³¹ is selected from the following groups: H, C₁₋₄ alkyls. In another preferred example, R³¹ is selected from the following group: H, methyl group.

In another preferred example, R³² and R³³ are independently selected from the following groups: H, C₁₋₄ alkyls.

In another preferred example, both R³² and R³³ are H.

In another preferred example, R³ is —N(R³²)R³³ or —C₁₋₄ alkylo-N(R³¹)R³² In another preferred example, R³ is —C₁₋₄ alkylo-N(R³¹)R³² In another preferred case, R³ is selected from the following groups: —NH₂, —CH₂—NH₂, —CH₂—N(CH3)₂, —CH₂—CH₂—NH₂, —CH₂—CH₂—N(CH₃)₂.

In another preferred example, R⁷ is an optional substitute group from the following groups: C₁₋₆ alkyl groups, C₃₋₁₀ naphthenic alkyl groups, 4 to 10 heterocyclic alkyl groups, C₃₋₁₀ cyclic alkyl groups, 4 to 10 member heterocyclic alkyl groups, C₆₋₁₀ aryl groups, and 5 to 10 member heteroaryl groups.

In another preferred example, R⁷ is an optional substitute for groups selected from the following groups: C₆₋₁₀ aryl groups, and 5 to 10 mixed aryl groups.

In another preferred example, in R⁷, the heteroaryl group comprises 1, 2 or 3 nitrogen heteroatoms as ring atoms, and the remaining ring atoms in the heteroaryl group are carbon atoms.

In another preferred example, R⁷ is optionally replaced by groups selected from the following groups:

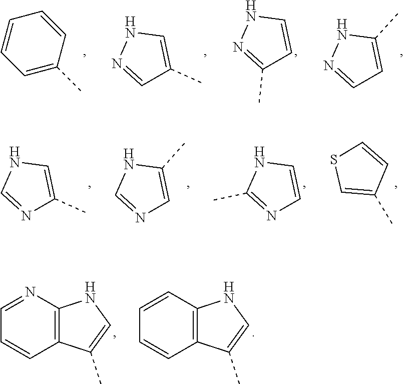

In another preferred example, R⁷ is an optionally substituted phenyl or 5-membered heteroaryl group.

In another preferred example, R⁷ is optionally replaced by groups selected from the following groups:

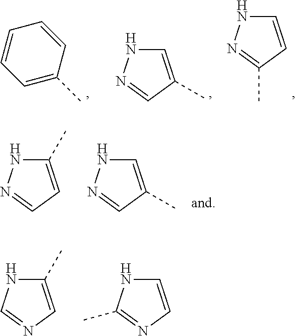

In another preferred example, R⁷, the optional substitution refers to the unsubstituted or group where 1 or 2 hydrogens in the group are replaced by the selected R substituent.

In another preferred example, R⁷ is selected from the following group:

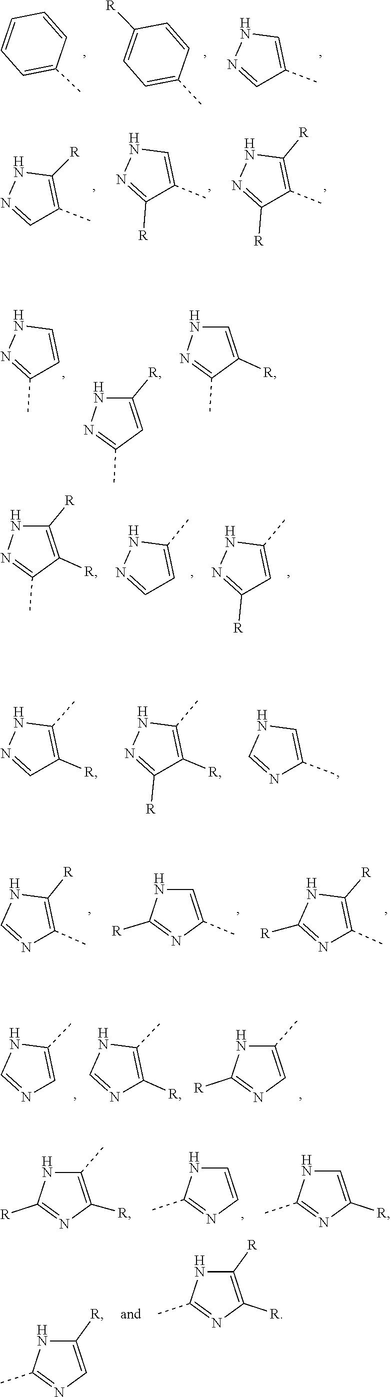

In another preferred example, in R′, R is independently selected from the following groups: C₁₋₆ alkyl group, —NR′R″; wherein each R′ is independently selected from the following groups: H, C₁₋₆ alkyl groups; Each R″ is selected from the following group: H, C₁₋₄ alkyls.

In another preferred example, ring A is the ring selected from the following group:

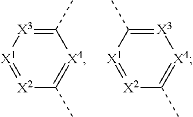

wherein X¹, X², X³ and X⁴ are independently selected from the following groups: CH and N.

In another preferred example, ring A is:

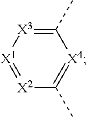

wherein X¹, X², X³ and X⁴ are independently selected from the following groups: CH and N.

In another preferred example, up to 1 or 2 of X¹, X², X³, and X⁴ are N.

In another preferred example, X¹, X², X³, and X⁴ are all CH.

In another preferred example, m1=0, 1, or 2; Better, m1=0 or 1; Better, m=0.

In another preferred case, the R^{A}s are R^{s1} or R^{A}R^{A1} independently; And R^{A}R^{A1} is selected from the following groups: halogens, optionally substituted C₁₋₆ alkyl groups, optionally substituted C₁₋₆ halogenated alkyl groups, and optionally substituted C₁₋₆ alkoxy groups (preferably, R^{A}R^{A1} is halogen).

In another preferred case, R^{A}s are H, C₁₋₄, or R^{A}R^{A1} independently; And R^{A}R^{A1} is selected from the following groups: halogens, optionally substituted C₁₋₆ alkyl groups, optionally substituted C₁₋₆ halogenated alkyl groups, and optionally substituted C₁₋₆ alkoxy groups (preferably, R^{A}R^{A1} is halogen).

In another preferred case, W² is —O— or —N(R⁸)—; Preferably, W² is —O— or —NH—.

In another preferred example, W² is —O—.

In another preferred example, R^{D}s are selected independently from the following groups: H, optionally substituted C₁₋₄ alkyl groups; Alternatively, the two R^{D}s and the carbon atoms attached to them work together to form an optionally substituted C₃₋₁₀ naphthenic group.

In another preferred example, R^{D}s are selected independently from the following groups: H, optionally substituted C₁₋₄ alkyl groups.

In another preferred case, R^{D} is selected independently from the following groups: H, methyl, and ethyl.

In another preferred case, R^{D} is both methyl groups.

In another preferred example,

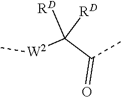

—O—C(R^{D})₂—C(O)—. In another preferred example,

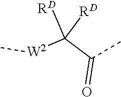

—O—C(CH₃)₂—C(O)—.

In another preferred example, R^{E} is selected from the following groups: H, methyl, and ethyl.

In another preferred case, R^{E} is H.

In another preferred example, R^{F}R^{F} is independently selected from the following groups: H, methyl and ethyl.

In another preferred example, R^{F}R^{F} is both H.

In another preferred case,

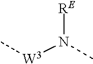

—C(O)—CH₂—NH—or —S(O)₂—NH—.

In another preferred example, in Formula I, ring A, ring B, ring C, L¹, W², W3, R^{A}, R^{B}, RC, R^{D}, R^{E}, R³, R⁷, R, subscript m1, subscript m2, subscript m3 and subscript m4 are each independently the corresponding groups in the example compounds or specific compounds in Tables A-II, B-II and C-II.

In another preferred example, in Equations I-P and G-2, rings A, X, X¹, X², X³, X⁴, X⁷, subscript o, subscript o2, subscript o3, subscript o4, W¹, W², W3, R^{A}, R^{B}, RC, R^{D}, R^{E}, R³, R′, R, subscript n1, subscript m1, subscript m2, Subscript m3 and subscript m4 are each independently the corresponding groups in the example compound or the specific compound in Tables A-II, B-II and C-II.

In another preferred example, in Equations I-3 and 1-4, rings A, X, X¹, X², X³, X⁴, subscript o1, subscript o2, subscript o3, subscript o4, W², W3, R^{A}, R^{B}, R^{C}, R^{D}, R^{E}, R³, R⁷, R, subscript m1, subscript m2, Subscript m3 and subscript m4 are each independently the corresponding groups in the example compound or the specific compound in Tables A-II, B-II and C-II.

In another preferred example, ring A, ring B, ring C, L¹, W², R^{A}, R^{B}, RC, R^{D}, R^{E}, R³, R⁷, R, subscript m1, subscript m2, subscript m3 can also be defined as in the first aspect.

In another preferred example, the compound is selected from Tables A-II

TABLE A-II

| Numerator | |
|---|---|
| number | structural formula |
| 115-010 | 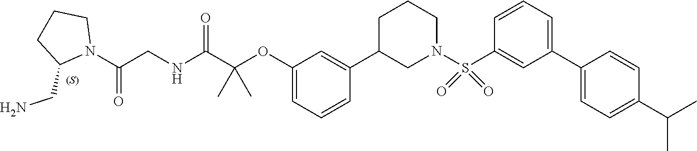 |
| 115-011 | 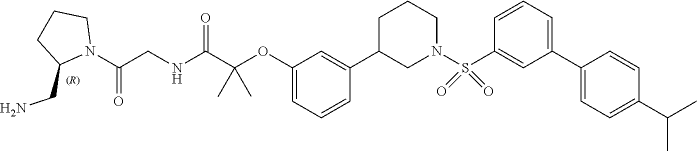 |
| 115-012 | 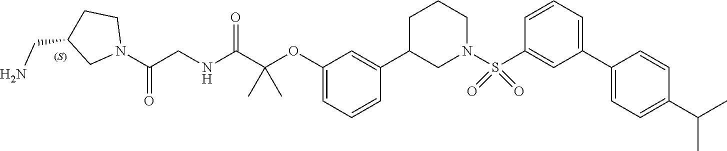 |
| 115-013 | 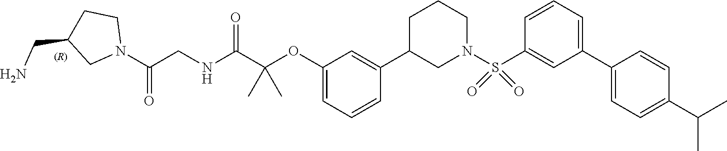 |
| 115-014 | 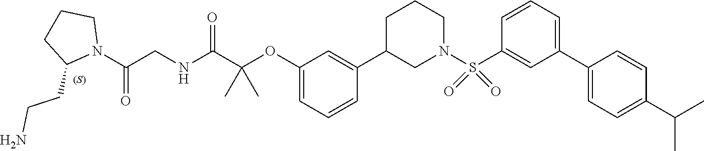 |
| 115-015 | 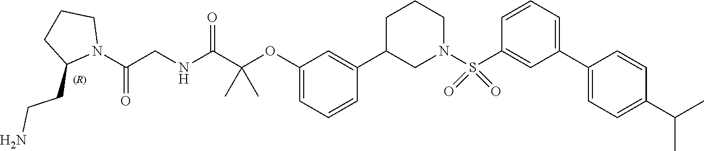 |
| 115-016 | 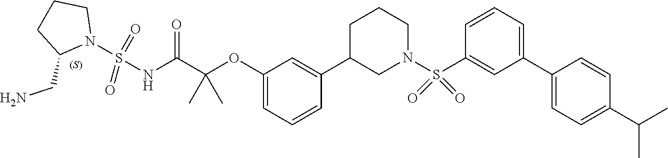 |
| 115-017 | 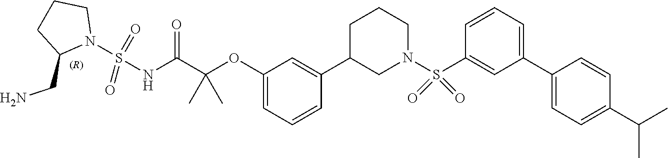 |
| 115-018 | 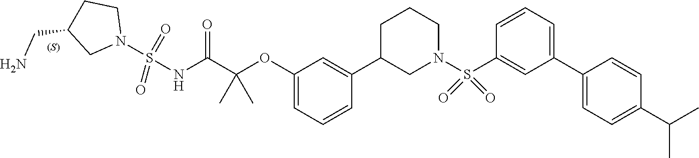 |
| 115-019 | 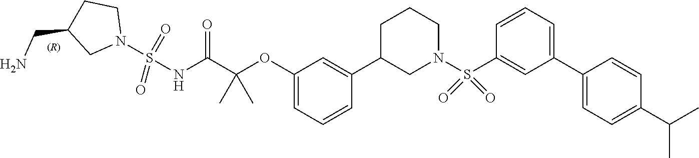 |
| 115-020 | 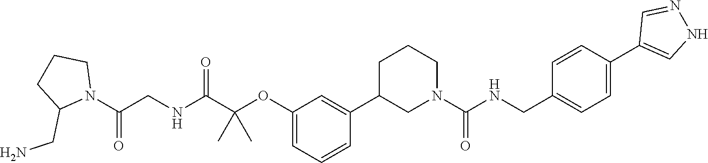 [SMILES: CC(C)(Oc1cccc(C2CCCN(C(=O)NCc3ccc(-c4cn[nH]c4)cc3)C2)c1)C(=O)NCC(=O)N1CCCC1CN] |
| 115-021 | 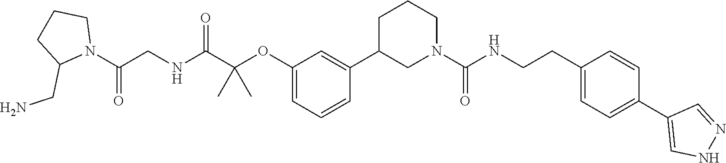 [SMILES: CC(C)(Oc1cccc(C2CCCN(C(=O)NCCc3ccc(-c4cn[nH]c4)cc3)C2)c1)C(=O)NCC(=O)N1CCCC1CN] |
| 115-022 | 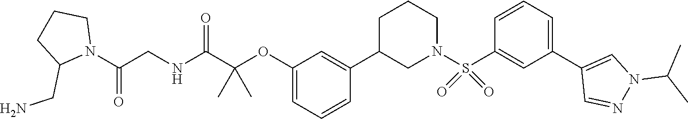 [SMILES: CC(C)n1cc(-c2cccc(S(=O)(=O)N3CCCC(c4cccc(OC(C)(C)C(=O)NCC(=O)N5CCCC5CN)c4)C3)c2)cn1] |
| 115-023 | 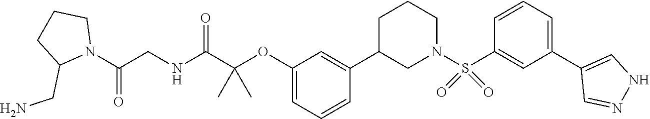 [SMILES: CC(C)(Oc1cccc(C2CCCN(S(=O)(=O)c3cccc(-c4cn[nH]c4)c3)C2)c1)C(=O)NCC(=O)N1CCCC1CN] |
| 115-027 | 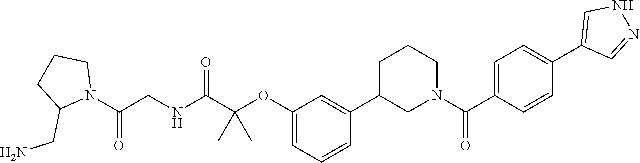 [SMILES: CC(C)(Oc1cccc(C2CCCN(C(=O)c3ccc(-c4cn[nH]c4)cc3)C2)c1)C(=O)NCC(=O)N1CCCC1CN] |
| 115-028 | 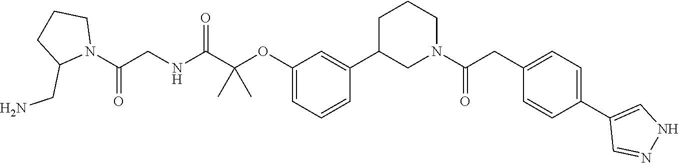 [SMILES: CC(C)(Oc1cccc(C2CCCN(C(=O)Cc3ccc(-c4cn[nH]c4)cc3)C2)c1)C(=O)NCC(=O)N1CCCC1CN] |
| 115-029 | 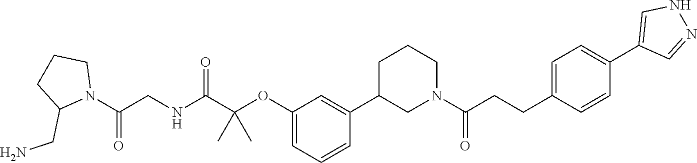 [SMILES: CC(C)(Oc1cccc(C2CCCN(C(=O)CCc3ccc(-c4cn[nH]c4)cc3)C2)c1)C(=O)NCC(=O)N1CCCC1CN] |
| 115-030 | 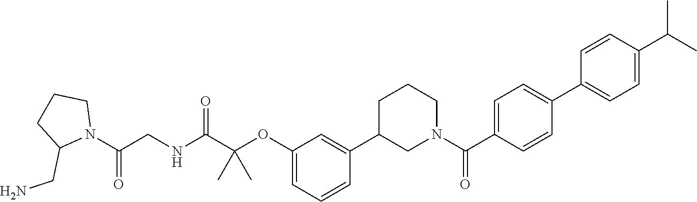 [SMILES: CC(C)c1ccc(-c2ccc(C(=O)N3CCCC(c4cccc(OC(C)(C)C(=O)NCC(=O)N5CCCC5CN)c4)C3)cc2)cc1] |
| 115-031 | 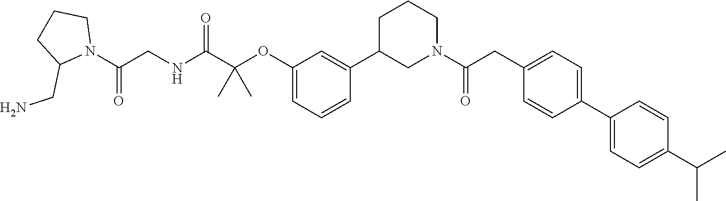 [SMILES: CC(C)c1ccc(-c2ccc(CC(=O)N3CCCC(c4cccc(OC(C)(C)C(=O)NCC(=O)N5CCCC5CN)c4)C3)cc2)cc1] |

In another preferred example, the compound is selected from Tables B-II

TABLE B-II

| Numerator | |
|---|---|
| number | structural formula |
| 115-046 | 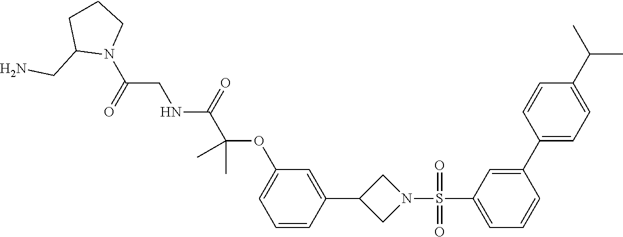 [SMILES: CC(C)c1ccc(-c2cccc(S(=O)(=O)N3CC(c4cccc(OC(C)(C)C(=O)NCC(=O)N5CCCC5CN)c4)C3)c2)cc1] |
| 115-047 | 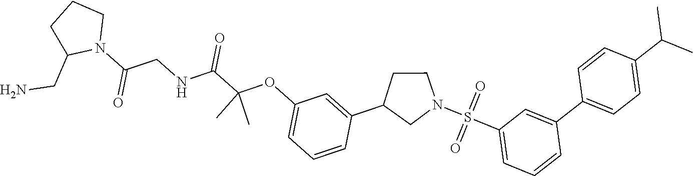 [SMILES: CC(C)c1ccc(-c2cccc(S(=O)(=O)N3CCC(c4cccc(OC(C)(C)C(=O)NCC(=O)N5CCCC5CN)c4)C3)c2)cc1] |
| 115-048 | 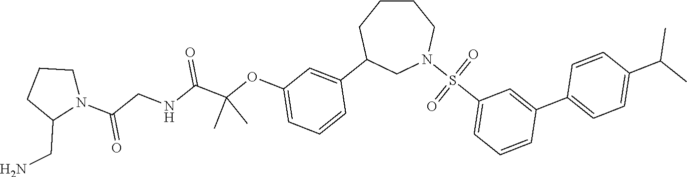 [SMILES: CC(C)c1ccc(-c2cccc(S(=O)(=O)N3CCCCC(c4cccc(OC(C)(C)C(=O)NCC(=O)N5CCCC5CN)c4)C3)c2)cc1] |
| 115-049 | 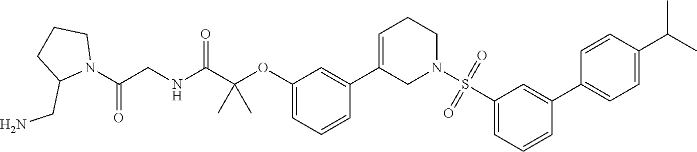 [SMILES: CC(C)c1ccc(-c2cccc(S(=O)(=O)N3CCC=C(c4cccc(OC(C)(C)C(=O)NCC(=O)N5CCCC5CN)c4)C3)c2)cc1] |
| 115-050 | 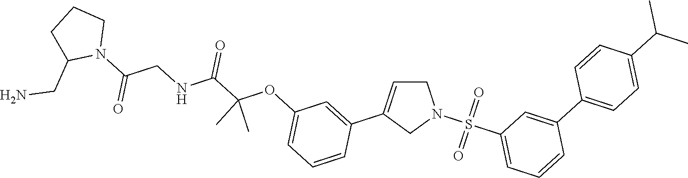 [SMILES: CC(C)c1ccc(-c2cccc(S(=O)(=O)N3CC=C(c4cccc(OC(C)(C)C(=O)NCC(=O)N5CCCC5CN)c4)C3)c2)cc1] |
| 115-051 | 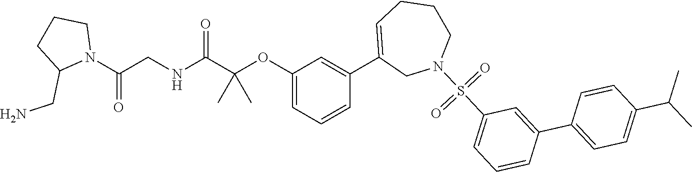 [SMILES: CC(C)c1ccc(-c2cccc(S(=O)(=O)N3CCCC=C(c4cccc(OC(C)(C)C(=O)NCC(=O)N5CCCC5CN)c4)C3)c2)cc1] |

In another preferred example, the compound is selected from Tables C-II

TABLE C-II

| Numera | |
|---|---|
| number | structural formula |
| 115-052 | 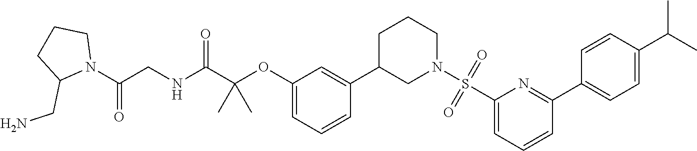 [SMILES: CC(C)c1ccc(-c2cccc(S(=O)(=O)N3CCCC(c4cccc(OC(C)(C)C(=O)NCC(=O)N5CCCC5CN)c4)C3)n2)cc1] |
| 115-053 | 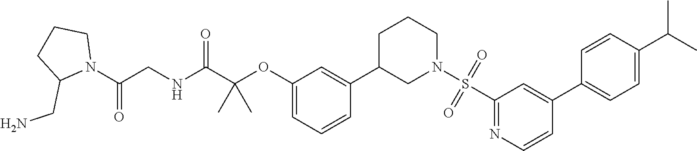 [SMILES: CC(C)c1ccc(-c2ccnc(S(=O)(=O)N3CCCC(c4cccc(OC(C)(C)C(=O)NCC(=O)N5CCCC5CN)c4)C3)c2)cc1] |
| 115-054 | 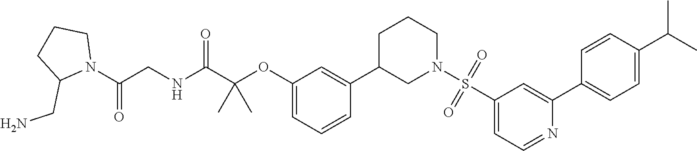 [SMILES: CC(C)c1ccc(-c2cc(S(=O)(=O)N3CCCC(c4cccc(OC(C)(C)C(=O)NCC(=O)N5CCCC5CN)c4)C3)ccn2)cc1] |
| 115-055 | 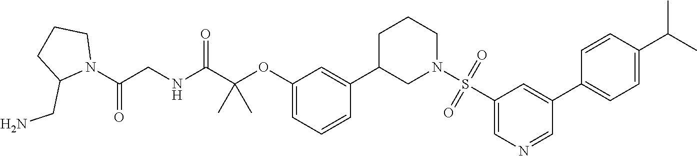 [SMILES: CC(C)c1ccc(-c2cncc(S(=O)(=O)N3CCCC(c4cccc(OC(C)(C)C(=O)NCC(=O)N5CCCC5CN)c4)C3)c2)cc1] |
| 115-056 | 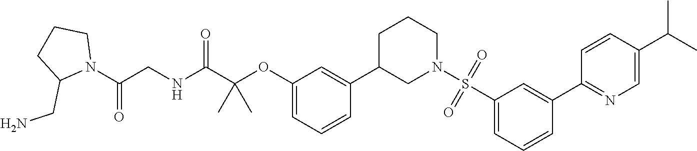 [SMILES: CC(C)c1ccc(-c2cccc(S(=O)(=O)N3CCCC(c4cccc(OC(C)(C)C(=O)NCC(=O)N5CCCC5CN)c4)C3)c2)nc1] |
| 115-057 | 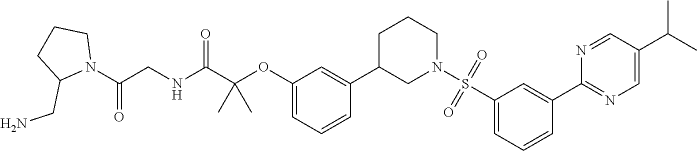 [SMILES: CC(C)c1cnc(-c2cccc(S(=O)(=O)N3CCCC(c4cccc(OC(C)(C)C(=O)NCC(=O)N5CCCC5CN)c4)C3)c2)nc1] |
| 115-058 | 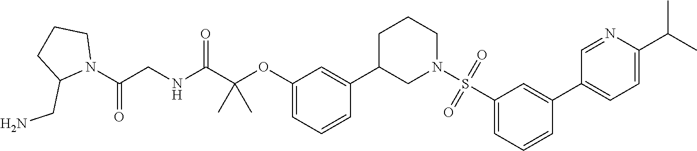 [SMILES: CC(C)c1ccc(-c2cccc(S(=O)(=O)N3CCCC(c4cccc(OC(C)(C)C(=O)NCC(=O)N5CCCC5CN)c4)C3)c2)cn1] |
| 115-059 | 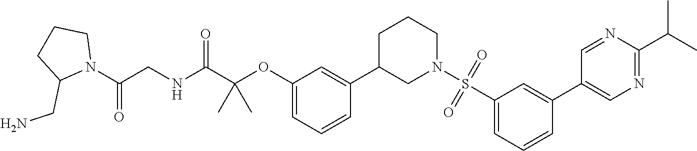 [SMILES: CC(C)c1ncc(-c2cccc(S(=O)(=O)N3CCCC(c4cccc(OC(C)(C)C(=O)NCC(=O)N5CCCC5CN)c4)C3)c2)cn1] |
| 115-060 | 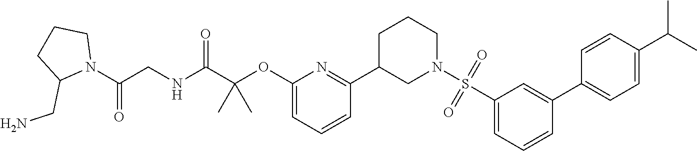 [SMILES: CC(C)c1ccc(-c2cccc(S(=O)(=O)N3CCCC(c4cccc(OC(C)(C)C(=O)NCC(=O)N5CCCC5CN)n4)C3)c2)cc1] |
| 115-061 | 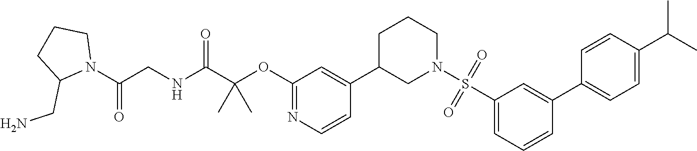 [SMILES: CC(C)c1ccc(-c2cccc(S(=O)(=O)N3CCCC(c4ccnc(OC(C)(C)C(=O)NCC(=O)N5CCCC5CN)c4)C3)c2)cc1] |
| 115-062 | 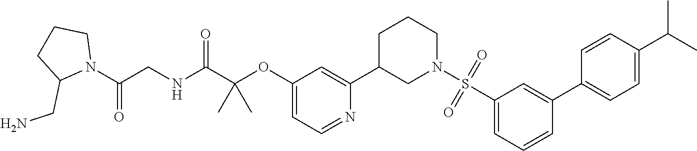 [SMILES: CC(C)c1ccc(-c2cccc(S(=O)(=O)N3CCCC(c4cc(OC(C)(C)C(=O)NCC(=O)N5CCCC5CN)ccn4)C3)c2)cc1] |
| 115-063 | 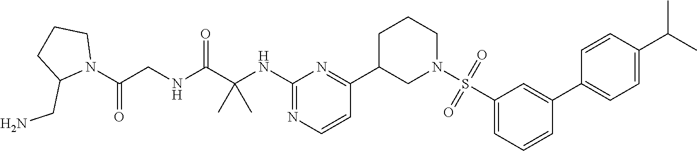 [SMILES: CC(C)c1ccc(-c2cccc(S(=O)(=O)N3CCCC(c4ccnc(NC(C)(C)C(=O)NCC(=O)N5CCCC5CN)n4)C3)c2)cc1] |
| 115-064 | 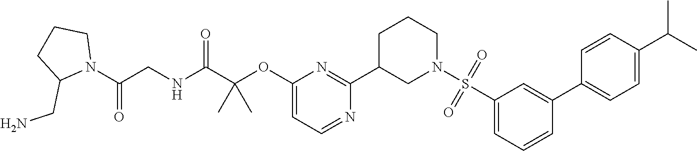 [SMILES: CC(C)c1ccc(-c2cccc(S(=O)(=O)N3CCCC(c4nccc(OC(C)(C)C(=O)NCC(=O)N5CCCC5CN)n4)C3)c2)cc1] |
| 115-065 | 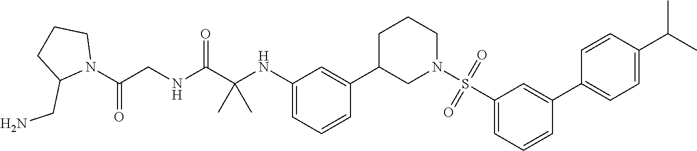 [SMILES: CC(C)c1ccc(-c2cccc(S(=O)(=O)N3CCCC(c4cccc(NC(C)(C)C(=O)NCC(=O)N5CCCC5CN)c4)C3)c2)cc1] |
| 115-066 | 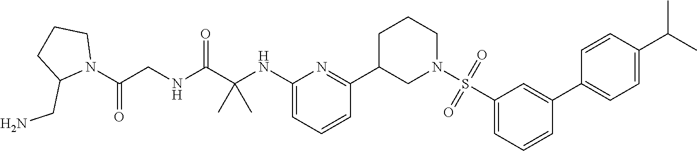 [SMILES: CC(C)c1ccc(-c2cccc(S(=O)(=O)N3CCCC(c4cccc(NC(C)(C)C(=O)NCC(=O)N5CCCC5CN)n4)C3)c2)cc1] |
| 115-067 | 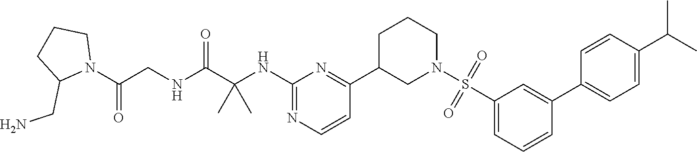 [SMILES: CC(C)c1ccc(-c2cccc(S(=O)(=O)N3CCCC(c4ccnc(NC(C)(C)C(=O)NCC(=O)N5CCCC5CN)n4)C3)c2)cc1] |
| 115-068 | 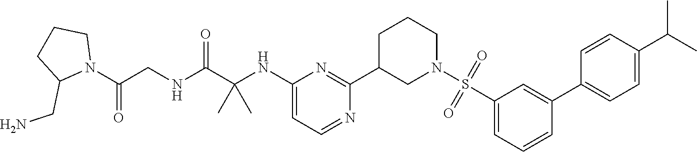 [SMILES: CC(C)c1ccc(-c2cccc(S(=O)(=O)N3CCCC(c4nccc(NC(C)(C)C(=O)NCC(=O)N5CCCC5CN)n4)C3)c2)cc1] |
| 115-069 | 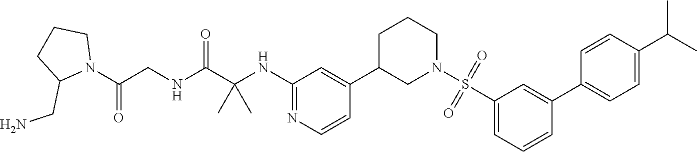 [SMILES: CC(C)c1ccc(-c2cccc(S(=O)(=O)N3CCCC(c4ccnc(NC(C)(C)C(=O)NCC(=O)N5CCCC5CN)c4)C3)c2)cc1] |

In the third aspect of the present invention, a drug composition is provided, comprising:
(i) the compound or its pharmaceutically acceptable salts, or its isomers, solvate, crystal forms or prodrugs as described in Section 1 or 2; With
(ii) Pharmaceutically acceptable carriers or excipients.

In the fourth aspect of the present invention, a compound or a pharmaceutically acceptable salt thereof, as described in the first or second aspect, or its isomers, solvate, crystal forms, or prodrugs are provided for the preparation of drugs for the treatment or prevention of diseases related to BCL9/β-catenin interactions.

In another preferred case, the diseases related to BCL9/β-catenin interactions include: cancer, tumors.

In the fifth aspect of the present invention, a method for treating or preventing diseases related to the interaction of BCL9/β-catenin is provided, comprising a procedure: administering a therapeutic effective amount of the compound or its pharmaceutically acceptable salt, or its isomer, solvent complex, crystal form or prodrug thereof, or a drug composition as described in the third side, to the object requiring it.

In another preferred case, the diseases related to BCL9/β-catenin interactions include: cancer, tumors.

In the sixth aspect of the present invention, a method for treating or preventing cancer is provided, comprising a procedure: administering a therapeutic effective dose of a compound or a pharmaceutically acceptable salt thereof as described in the first or second aspects, or its isomers, solvate, crystal forms or prodrugs, or a drug composition as described in the third side.

In the seventh aspect of the present invention, a compound or its pharmaceutically acceptable salt, or its isomer, solvent complex, crystal form, or prodrug used in the preparation of drugs for the treatment or prevention of fibrosis or related diseases, as described in the first or second aspects, is provided.

In another preferred case, fibrosis or related disorders include: pulmonary fibrosis, liver fibrosis, nonalcoholic hepatic steatohepatitis, bone fibrosis, or a combination thereof.

In the eighth aspect of the present invention, a method for treating or preventing fibrosis-related diseases is provided, comprising a procedure: administering a therapeutic effective amount of a compound or a pharmaceutically acceptable salt thereof as described in the first or second aspects, or an isomer thereof, a solvent complex, a crystal form or a prodrug, or a drug composition as described in the third side.

In another preferred case, fibrosis or related disorders include: pulmonary fibrosis, liver fibrosis, nonalcoholic hepatic steatohepatitis, bone fibrosis, or a combination thereof.

In the ninth aspect of the present invention, a binding of BCL9 to β-catenin in the object is provided; and/or modulate Wnt/β-catenin signaling in the subject; and/or reduced regulatory T cell survival in the subject; and/or reduce the expression of VEGF in the tumor in the subject; and/or increase the infiltrating CD4+ T cells and CD8+ T cells in the tumor in the subject; and/or increase T helper 17 (Th17) cells in tumors entering the subject; and/or reduce dendritic cells in tumors in the subject; and/or when administered to the subject, make the half-life (T 112) greater than 2 hours; and/or induce a tumor microenvironment conducive to an immune response in the subject; and/or inhibits tumor growth in the subject; and/or inhibition of the proliferation of cancer stem cells in the subject; and/or methods of inhibiting tumor metastasis in the subject, including the procedure: administering to the subject the compound described in Aspect 1 or Aspect 2 or its pharmaceutically acceptable salts, or its isomers, solvate, crystal forms, or prodrugs, or drug compositions as described in Aspect 3, or exposing the subject to the compounds described in Aspects 1 or 2 or their pharmaceutically acceptable salts, or their isomers, solvate, crystal forms, or prodrugs.

In another preferred example, the object is a mammal, preferably a human.

In another preferred example, the object is a cell.

In another preferred case, the method described is non-therapeutic in vitro.

It should be understood that within the scope of the present invention, the above technical features of the present invention and the technical features specifically described below (such as embodiments) can be combined with each other to form a new or preferred technical solution. Due to space limitations, I will not repeat them one by one here.

### Illustrated Description

not

### Specific Embodiment

After extensive and in-depth research, the inventor unexpectedly discovered a class of small molecule compounds with novel structures that superior to the activity of inhibiting the interaction between BCL9 and β-catenin. In addition, the inventor found that this compound has excellent therapeutic and preventive effects in fibrosis and related diseases. Based on this, the inventor completed the invention.

### TERM

Unless otherwise specified, terms or abbreviations in this document have a general meaning as understood by those skilled in the field.

Unless otherwise noted, in this article, a single bond in the compound structure is represented by a dotted line ( [symbol] ) to represent the junction position to the rest of the molecule.

As used herein, the terms “contains”, “contains”, or “includes” indicate that the various components may be applied together to a mixture or composition of the invention. Therefore, the term “is mainly made up of . . . composed” and “composed of . . . composition” is included in the term “contains”.

Unless otherwise noted, the term “alkyl”, by itself or as part of another substituent, refers to a linear or branched-chain hydrocarbon group with a specified number of carbon atoms (i.e., C₁₋₆ represents 1-6 carbons). Examples of alkyl groups include methyl groups, ethyl groups, n-propyl groups, isopropyl groups, n-butyl groups, tert-butyl groups, isobutyl groups, secondary butyl groups, n-pentyl groups, n-hexyl groups, n-heptyl groups, n-octyl groups, etc.

The term “alkenyl” refers to an unsaturated alkyl group with one or more double bonds. Similarly, the term “alkyl group” refers to an unsaturated alkyl group with one or more triple bonds. In general, the alkene group has 1-6 carbon atoms (i.e., C₁₋₆ alkene group), and the alkyne group has 1-6 carbon atoms (i.e., C₁₋₆ alkyne group). Examples of such unsaturated alkyl groups include: vinyl, 2-propylyl, croton, 2-isoprenyl, 2-(butadieenyl), 2,4-pentadienyl, 3-(1,4-pentadienyl), acetylene, 1- and 3-propynyl, 3-butynyl, and more advanced homologues and isomers.

The terms “alkoxy”, “alkylamino” and “alkyl-sulfide” (or thioalkoxy) are used in their regular sense to refer to those alkyl groups that are attached to the rest of the molecule by oxygen, amino or sulfur atoms, respectively. In addition, for dialkyl aminos, the alkyl moiety can be the same or different, and can also be combined with nitrogen atoms linked to each alkyl group to form a 3-7 membered ring. Therefore, the group representation of —NRaRb includes piperidine groups, pyrrolide groups, morpholine groups, azetidinyl, etc.

As used herein, the term “alkyl group”, either by itself or as part of another substituent, refers to a divalent group derived from an alkane, such as —CH₂—, —CH₂CH₂—.

As used herein, the term “aminoalkyl group” refers to the replacement of 1 or 2 hydrogens by an amino group in an alkyl group defined above with a specified number of carbon atoms. For example, —(CH2)_{2NH2}.

As used herein, the term “naphthenic group” refers to a saturated hydrocarbon ring with a specified number of ring atoms (e.g., C₃₋₁₀ naphthenyl, preferably C₃₋₆ naphthenic group). “Naphthenic groups” can be monocyclic (such as cyclopropyl, cyclobutyl, cyclohexyl, etc.), or bicyclic and polycyclic hydrocarbon rings (including parallel rings, spiral rings, bridge rings, etc.), such as cyclopropylene, cyclobutene, cyclopentene, cyclopentadiene, etc. The term “heterocyclic alkyl group” refers to a naphthenic group containing one to five (preferably 1, 2, 3, or 4) heteroatoms selected from N, O, and S, where nitrogen and sulfur atoms are optionally oxidized and nitrogen atoms optionally quaternalized. Heterocyclic alkyl groups can be single-ring, double-ring, or multi-cyclic systems (including parallel rings, spiral rings, bridge rings, etc.). In general, heterocyclic groups usually include 4-10 ring atoms (i.e., 4 to 10-membered heterocyclic groups), preferably 4-7 (e.g., 4, 5, 6) ring atoms (i.e., 4 to 7-membered heterocyclic groups, or 4 to 6-membered heterocyclic groups) and contain 1, 2, 3, or 4 (preferably 1 or 2) heterocyclic atoms. Non-restrictive examples of heteronacyclic alkyl groups include pyrroldine, imidazolidine, pyrazodine, butyrolactam, penolactam, imidazolidone, hydantoin, dioxenimide, piperidine, 1,4-dioxane, morpholine, thiomorpholine, thiomorpholine-S-oxide, thiomorpholine-S, S-oxide, piperazine, pyran, pyridinone, 3-pyrroline, thianosulfon, pyranone, tetrahydrofuran, tetrahydrothiophene, quinine ring, etc. Heterocyclic alkyl groups can be attached to the rest of the molecule by ring carbon or heteroatoms such as cyclic nitrogen.

As used herein, the term “cyclene group”, used alone or as part of a group, refers to a cyclic hydrocarbon with a specified number of cyclic atoms (e.g., C₃₋₁₀ cyclene group, or C₃₋₆ cyclene group) with 1 or 2 double bonds (preferably only 1 double bond) between the ring vertices. “Cyclogenic group” can be a single ring, or it can refer to bicyclic and polycyclic hydrocarbon rings (including parallel rings, spiral rings, bridge rings, etc.). Examples of cyclopentene groups include, for example, cyclopropylene, cyclobene, cyclopentene, cyclopentadiene, etc. Similarly, the term “heterocyclic alkene group” refers to a cyclene group containing 1 to 5 (preferably 1, 2, 3, or 4) heteroatoms selected from N, O, and S, where nitrogen and sulfur atoms are optionally oxidized and nitrogen atoms optionally quaternalized. Heterocyclic alkene groups can be single-ring, bi-ring, or multi-cyclic systems (including parallel rings, spiral rings, bridge rings, etc.). In general, heterocyclic alkyl groups usually include 4-10 cyclic atoms (i.e., 4 to 10 heterocyclic groups), preferably 4-7 (e.g., 4, 5, 6) cyclic atoms (i.e., 4 to 7 heterocyclic groups, or 4 to 6 heterocyclic groups) and contain 1, 2, 3, or 4 (preferably 1 or 2) heterocyclic atoms.

For terms such as naphthenic alkyl (alkyl groups) and heterocycloalkyl alkyl groups (alkyl groups), they refer to naphthenic or heterocyclic alkyl groups connected to the rest of the molecule by alkyl or alkyl linkers. For example, cyclobumylmethyl—is a cyclobyan-butyl ring attached to the methylene linker group attached to the rest of the molecule.

Unless otherwise noted, the term “aryl” denotes a polyunsaturated (usually aromatic) hydrocarbon group that can be monocyclic or multicyclic (up to tricyclics) bonded together or covalently linked. In general, aryl groups have 6-10 ring atoms. The term “heteroaryl” refers to an aryl group (or ring) containing 1 to 5 heteroatoms selected from N, O, and S, in which nitrogen and sulfur atoms are optionally oxidized and nitrogen atoms are optionally quaternally ammonized. Generally, heteroaryl groups have 5-10 cyclic atoms, i.e., 5-10 heteroaryl groups, preferably, 5-6 cyclic atoms, i.e., 5-6 heteroaryl groups, and contain 1, 2, 3, or 4 heteroatoms. Heteroaryl groups can be attached to the rest of the molecule by heteroatoms. Non-restrictive examples of aryl groups include phenyl, naphthalene, and biphenyl, while non-restrictive examples of heteroaryl groups include pyridine, dyazine, pyrazine, pyrimidine, triazine, quinoline, quioline, quinozoline, isoline, phthalazine, benzotriazinyl, purine, benzimidazole, benzopyrazole, benzotriazole, benzoisooxazole, isobenzofuryl, isoindole, mesazatonyl, benzotriazine, thiophenoxypyridyl, thiophenoxypyrimidine, pyrazoxypyrimidyl, imidazopyridine, benzothiazole, benzofuranyl, benzothiophenyl, indoleyl, quinoline, isoquinoline, isothiazoyl, pyrazoleyl, indiazolyl, episidyl, imidazolyl, triazoleyl, tetraazoleyl, oxazoleyl, isooxazoleyl, thiadiazolyl, pyrroyl, thiazolyl, furanyl, thiophene group, etc.

The substituents of the above aryl and heteroaryl ring systems are selected from the following groups of acceptable substituents.

For brevity, the term “aryl” is used in combination with other terms (e.g., aryl groups, aryl sulfur groups, aryl groups) to include aryl and heteroaryl rings as defined above. Thus, the term “aryl group” refers to those groups in which the aryl group is attached to an alkyl group attached to the rest of the molecule (e.g. benzyl group, phenylethyl group, pyridine methyl, etc.).

In some embodiments, the terms described above (e.g., “alkyls”, “aryls” and “heteroarylgroups”) will include substituted and unsubstituted forms of the designated group. The preferred substituents for each type of group are provided below. For brevity, the terms aryl and heteroaryl groups will refer to the substituted or unsubstituted forms provided below, while the terms “alkyls” and related aliphatic groups refer to unsubstituted forms, unless substituted are specified.

Substituents of alkyl groups (including those commonly called alkyl groups, alken groups, alkyl groups, and naphthenic alkyl groups) can be selected from various groups of the following groups: -halogens, —OR′, —NR′R′, —SR′, —SiR′R″, OC(O)R′, —C(O)R′, —CO₂R′, —CONR′R″, —OC(O)NR′R′, —NR′, —NR′—C(O)NR″, —NR′, —NR′C(O)₂R′, —S(O)R′, —S(O)₂R′, —S(O)₂NR′R″, NR′S(O)₂R″, —CN and —NO₂, from zero to (2m′+1), where m′ is the total number of carbon atoms in this group. R′, R′, and R″ each independently represent hydrogen, unsubstituted Cl-8 alkyl groups, unsubstituted heteroalkyl groups, unsubstituted aryl groups, aryl groups substituted by 1-3 halogens, unsubstituted C₁₋₈ alkyl groups, C₁₋₈ alkoxy groups or C₁₋₈ thioalkoxy groups, or unsubstituted aryl-C₁₋₄ alkyl groups. When R′ and R′ are attached to the same nitrogen atom, they can combine with the nitrogen atom to form a 3-, 4-, 5-, 6-, or 7-ring. For example, “—NR′R” refers to the inclusion of 1-pyrrolil and 4-morpholine groups.

The term “acyl”, used alone or as part of another group, refers to the substituent ═O substitution of two substituents on the carbon closest to the junction point of that group (e.g., —C(O)CH₃, —C(O)CH₂CH₂OR′, etc.).

Similarly, the substituents of aryl and heteroaryl groups are diverse and are often selected from: -halogens, —OR′, —OC(O)R′, —NR′R″, —SR′, —R′, —CN, —NO₂, —CO₂R′, CONR′R′, C(O)R′, —OC(O)NR′, —NR″C(O)R′, —NR″C(O)₂R′, —NR′—C(O)NR″, —S(O) R′, —S(O)₂R′, S(O)₂NR′R″, NR′S(O)₂R″, N3, perfluoro(C₁-C₄) alkoxy and perfluoro(C₁-C₄) alkyl groups, from zero to the total number of open valences on the aromatic ring system; R′, R′ and R″ are independently selected from hydrogen, C₁₋₈ alkyl group, C₃₋₆ naphthenic alkyl group, C₂₋₈ alkyl group, C₂₋₈ alkyl group, unsubstituted aryl group and heteroaryl group, (unsubstituted aryl group)-C₁₋₄ alkyl group and unsubstituted aryl group-C₁₋₄ alkyl group. Other suitable substituents include each of the above aryl substituents attached to the ring atom by an alkyl chain of 1-4 carbon atoms.

As used herein, the term “heteroatom” is intended to include oxygen (O), nitrogen (N), sulfur (S), and silicon (Si).

As used in this article, “halogens” refer to F, Cl, Br, and I. Better, halogen atoms are selected from F, Cl, and Br.

For the compounds presented herein, the bond from the substituent (usually the R group) to the center of the aromatic ring (e.g., benzene, pyridine, etc.) will be understood to refer to the bond that provides a connection at any available vertex of the aromatic ring. In some embodiments, the description also includes connections on the rings of the aromatic ring. For example, a key drawn to the center of the indole benzene portion will represent a bond connected to any available vertices in the six- or five-membered ring portion of indole.

Unless otherwise specified, all compounds appearing in the present invention are intended to include all possible optical isomers, such as a single chiral compound, or a mixture of various different chiral compounds (i.e., racemic). Among all compounds of the present invention, each chiral carbon atom can be optionally R or S or a mixture of R and S configurations. Preferably, in this article, unless specifically stated, when a single bond is represented in a compound structure/, the compound includes compounds with a single configuration of the S or R configurations, or a mixture of the S and R configurations (such as racemic).

### Active Ingredients:

As used herein, the term “compound of the present invention” refers to the compound described in the first aspect of the present invention. The term also includes various crystal forms of compounds described in the first aspect of the present invention, pharmaceutically acceptable salts, hydrates or solvate.

As used herein, the term “pharmaceutically acceptable” ingredient refers to a substance that is suitable for use in humans and/or animals without undue adverse reactions (e.g., toxicity, irritation, and allergic reactions), i.e., with a reasonable benefit/risk ratio.

As used herein, the term “therapeutic effective dose” refers to any amount of a drug that, when used alone or in combination with another therapeutic agent, promotes resolution of the disease, which manifests as a decrease in the severity of disease symptoms, an increase in the frequency and duration of symptom-free periods, or prevention of disability or disability caused by the disease. The “effective therapeutic dose” of the drug of the present invention also includes the “effective dose for prevention”, which is any amount of the drug described below, which can inhibit the occurrence or recurrence of the disease when administered alone or in combination with another therapeutic agent to an object at risk of developing disease or suffering from disease recurrence.

The term “pharmaceutically acceptable salts” is intended to include salts prepared from active compounds and relatively non-toxic acids or bases, depending on the specific substituents on the compounds described herein. When the compound of the present invention contains a relatively acidic functional group, the alkali addition salt can be obtained by contacting such compounds in neutral form with a sufficient amount of the desired base (either solvent-free or in a suitable inert solvent). Examples of salts derived from pharmaceutically acceptable inorganic bases include aluminum, ammonium, calcium, copper, iron, ferrous iron, lithium, magnesium, manganese, manganese, manganese, potassium, sodium, zinc, etc. Salts derived from pharmaceutically acceptable organic bases include salts of primary, secondary and tertiary amines, including substituted amines, cyclic amines, naturally occurring amines, etc., such as arginine, betaine, caffeine, choline, N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hybamine, Isopropylamine, lysine, meglucosamine, morpholine, piperazine, piperidine, polyamine resin, procaine, purine, theobromine, triethylamine, trimethylamine, tripropylamine, aminobutamine, etc. When the compound of the present invention contains a functional group of relatively alkaline, acid-added salts can be obtained by contacting such compounds in neutral form with a sufficient amount of the desired acid (either solvent-free or in a suitable inert solvent). Examples of pharmaceutically acceptable acid-added salts include those derived from inorganic acids, such as hydrochloric acid, hydrobromic acid, nitric acid, carbonic acid, monohydrocarbonic acid, phosphoric acid, monohydrophosphoric acid, dihydrophosphoric acid, sulfuric acid, monohydrosulfuric acid, hydroiodic acid, or phosphoric acid, etc.; and salts derived from relatively non-toxic organic acids, such as acetic acid, propionic acid, isobutyric acid, malonic acid, benzoic acid, succinic acid, caprylic acid, transbutylene acid, mandelic acid, benzene sulfonic acid, p-toluene sulfonic acid, citric acid, tartaric acid, mesysulfonic acid, etc. It also includes salts of amino acids, such as arginine, and salts of organic acids, such as glucuronic acid or galactunoric acid (see, e.g., Berge, S. M., et al., “Pharmaceutical Salts”, Journal of Pharmaceutical Science, 1977, 66, 1-19). Some specific compounds of the present invention contain both basic and acidic functional groups, so that the compounds can be converted into alkali-added salts or acid-added salts.

The neutral form of the compound can be regenerated by contacting salts with bases or acids and separating the parent compound in a conventional manner. The parent form of a compound differs from the various salt forms in certain physical properties (e.g., solubility in polar solvents), but nevertheless, for the purposes of the present invention, those salts are equivalent to the parent form of the compound.

In addition to the salt form, the present invention provides a compound in the form of a prodrug. Prodrugs for compounds described herein are those that are readily subject to chemical changes under physiological conditions to provide the compound of the present invention. In addition, the prodrug can be transformed into a compound of the present invention by chemical or biochemical methods in an ex vivo environment. For example, when placed in a transdermal patch container containing a suitable enzyme or chemical agent, the prodrug can be slowly transformed into a compound of the present invention.

Some compounds of the present invention can exist in non-solvable form as well as solvated form (i.e., solvate), including hydrated forms (i.e., hydrates). The solvable form is usually equivalent to the non-solvable form and should be included in the scope of the invention. Some compounds of the present invention can exist in polymorphic or amorphous form. In general, all physical forms are equivalent for the application considered by the present invention and should be included in the scope of the present invention.

Some compounds of the present invention possess asymmetric carbon atoms (optical centers) or double bonds; Racemoners, amaniomers, geometric isomers, regional isomers, and separate isomers (e.g., isolated enantiomers) shall be included within the scope of the invention. When the compounds presented herein have a definite stereochemistry (denoted as R or S, or indicated by a dotted line or wedge bond), those compounds will be understood by experts in the field to be substantially free of other isomers (e.g., at least 80%, 90%, 95%, 98%, 99% and up to 100% without other isomers).

The compound of the present invention may also contain an unnatural proportion of atomic isotopes at one or more isotope atoms constituting such compounds. The unnatural ratio of an isotope can be defined as the amount from the amount naturally found in the atom in question to the amount of 100% of that atom. For example, compounds can be incorporated with radioactive isotopes such as tritium (³H) iodine-125 (¹²⁵I) or carbon-14 (¹⁴C) or non-radioactive isotopes such as deuterium (²H) or carbon-13 (¹³C) In addition to those used in this application, such isotope variants may provide additional uses. For example, isotopic variants of the compound of the present invention may have additional uses, including but not limited to as diagnostic and/or imaging reagents, or as cytotoxic/radiotoxic therapeutic agents. In addition, isotopic variants of the compounds of the present invention may have altered pharmacokinetic and pharmacodynamic profiles that contribute to increasing safety, tolerability, or efficacy during treatment. All isotopic variants of the compound of the present invention shall be included in the scope of the present invention, regardless of whether they are radioactive or not.

### Drug Composition and Method of Administration

Due to the excellent interaction between BCL9/β-catenin and egg BCL9/β-catenin (BCL9/-catenin PPI). Therefore, the compound of the present invention and its various crystal forms, pharmaceutically acceptable inorganic or organic salts, hydrates or solvate, and pharmaceutical compositions containing the compound of the present invention as the main active ingredient may be used to treat, prevent, and alleviate diseases caused by the interaction of BCL9/β-catenin. According to the prior art, the compound of the present invention can be used to treat the following diseases: cancer, tumors, etc., for example, familial adenomatous polyposis (FAP), eye cancer, rectal cancer, colon cancer, colorectal cancer, cervical cancer, prostate cancer, breast cancer, bladder cancer, oral cancer, benign and malignant tumors, stomach cancer, liver cancer, pancreatic cancer, lung cancer, uterine body, Ovarian Cancer, Prostate Cancer, Testicular Cancer, Kidney Cancer, Brain/CNS Cancer, Laryngeal Cancer, Multiple Myeloma, Cutaneous Melanoma, Acute Lymphoblastic Leukemia, Acute Myeloid Leukemia, Ewing's Sarcoma, Kaposi's Sarcoma, Basal Cell Carcinoma and Squamous Cell Carcinoma, Small Cell Lung Cancer, Chorionic Carcinoma, Rhabdomyosarcoma, Hemangiosarcoma, Angioendothelioma, Wilm's Tumor, Neuroblastoma, Oral/Pharyngeal Cancer, Esophageal Cancer, Laryngeal Cancer, Lymphoma, Neurofibromatosis, tuberous sclerosis, hemangioma, gastric cancer, ovarian cancer, hepatocellular carcinoma, lymphatic vessels, etc.

In addition, the compound of the present invention also has excellent ability to treat fibrosis, therefore, the compound of the present invention and its various crystal forms, pharmaceutically acceptable inorganic or organic salts, hydrates or solvate, and pharmaceutical compositions containing the compound of the present invention as the main active ingredient may be used to treat, prevent, and alleviate fibrosis and various diseases associated with fibrosis. Fibrosis can occur in a variety of organs, and the main pathological changes are an increase in fibrous connective tissue and a decrease in parenchymal cells in organ tissues.

Example diseases of fibrosis and its associated diseases are as follows:

| Major organs | Typical diseases and syndromes |
|---|---|
| 1 Lungs | Diseases with known causes: inorganic dust occupational diseases (silicosis, asbestosis, coal |
| | lung, etc.); organic dust and hypersensitivity pneumonia (farmers' lungs, air-conditioning |
| | humidifier lungs, pigeon feeder's lungs, bagasse pneumoconiosis, etc.); |
| | Drug/treatment-related diseases (antibiotics, NSAIDs, cardiovascular drugs, antineoplastic |
| | drugs, oral hypoglycemic drugs, oxygen, morphine, etc.); Infectious diseases (tuberculosis, |
| | viral pneumonia, pneumocystis infection, etc.) Secondary lung diseases (left heart failure, |
| | congenital heart disease, adult respiratory distress syndrome, chronic cardiac |
| | insufficiency-related, transplant rejection-related lung diseases, etc.); Diseases of unknown |
| | etiology: pulmonary primary diseases (idiopathic interstitial pneumonia, bronchiolitis |
| | obliterans with organizing pneumonia, pulmonary lymphangioleiomyoma, etc.); Lung |
| | diseases associated with collagen vascular disease (systemic lupus erythematosus, |
| | rheumatoid arthritis, progressive system sclerosis, polymyositis, dermatomyositis, mixed |
| | connective tissue disease, etc.); Alveolar filling disease (diffuse alveolar hemorrhage |
| | syndrome, alveolar proteinosis, eosinophilic pneumonia, pulmonary vasculitis, lymphocytic |
| | interstitial pneumonia, necrotizing nodular granulomas, familial pulmonary fibrosis, etc.). |
| 2. | ischemic heart disease (replacement and interstitial fibrosis after myocardial infarction); |
| Cardiovascular | hypertensive heart disease; inflammatory cardiomyopathy (viral myocarditis); Metabolic |
| system | cardiomyopathy (hemochromatous cardiomyopathy, amyloidosis cardiomyopathy, glycogen |
| | accumulation cardiomyopathy, diabetic cardiomyopathy, etc.); Keshan disease; Dilated |
| | cardiomyopathy; hypertrophic cardiomyopathy, restrictive cardiomyopathy; arrhythmogenic |
| | right ventricular cardiomyopathy, etc |
| 3. | viral cirrhosis (hepatitis B, C and D viral hepatitis); schistosomiasis cirrhosis; alcoholic |
| Liver | cirrhosis; Biliary cirrhosis (primary biliary cirrhosis, secondary gallstones, periportaltitis); |
| | metabolic cirrhosis (hepatolenticular degeneration, hemochromatosis); toxic cirrhosis |
| | (organophosphorus poisoning, carbon tetrachloride poisoning, hepatotoxic drugs such as |
| | isoniazid, tetracycline, chlorpromazine poisoning, etc.); dystrophic cirrhosis; Cardiogenic |
| | cirrhosis (chronic congestive heart failure). |
| 4 | acute pancreatitis; Pancreatic duct obstruction; chronic alcoholism; Oddi sphincter |
| Pancreas | dysfunction; pancreatic ischemia, etc. |
| 5 | vascular (hypertension); Immune (glomerulonephritis, systemic lupus erythematosus, |
| Kidneys | scleroderma, renal transplant rejection) infectious (pyelonephritis, kidney stones) metabolic |
| | (hyperlipidemia, diabetes, hyperuriuria, hypercalciuria etc |
| 6. Spleen | Splenic fibroproliferative disease |
| 7 Eyes | After eye trauma and surgery, diabetic retinotic membrane fibrous hyperplasia |
| 8 Nervous | Post-spinal cord trauma, stroke scarring, Alzheimer's disease |
| system | |
| 9 Bone | Idiopathic and drug-induced myelofibrosis, polycythemia vera, chronic myeloid leukemia, |
| marrow | Hodgkin's disease |

The drug composition of the present invention comprises the compound of the present invention within the safe and effective amount range or its pharmacologically acceptable salt and pharmacologically acceptable excipients or carriers. The “safe and effective amount” refers to the amount of the compound that is sufficient to significantly improve the condition without serious side effects. Typically, the drug composition contains 1-2000 mg of the compound/dose of the invention, and better, 10-500 mg of the compound/dose of the invention. Preferably, the “one dose” is a capsule or tablet.

“Pharmaceutically acceptable carrier” means one or more compatible solid or liquid fillers or gel substances that are suitable for human use and must be of sufficient purity and low enough toxicity. “Compatibility” here refers to the ability of the components in the composition to be mixed with the compounds of the present invention and between them without significantly reducing the efficacy of the compounds. Some examples of pharmaceutically acceptable carriers are cellulose and its derivatives (e.g., sodium carboxymethylcellulose, sodium ethylcellulose, cellulose acetate, etc.), gelatin, tale, solid lubricants (e.g., stearic acid, magnesium stearate), calcium sulfate, vegetable oils (such as soybean oil, sesame oil, peanut oil, olive oil, etc.), polyols (such as propylene glycol, glycerin, mannitol, sorbitol, etc.), emulsifiers (such as Tween R), wetting agents (such as sodium lauryl sulfate), colorants, flavorings, stabilizers, antioxidants, preservatives, non-pyrogenic water, etc.

There are no special restrictions on the mode of administration of the compound or drug composition of the present invention, and representative administration methods include (but are not limited to): oral, intratumoral, rectal, parenteral (intravenous, intramuscular, or subcutaneous), and topical administration.

Solid dosage forms for oral administration include capsules, tablets, pills, powders, and granules. In these solid dosage forms, the active compound is mixed with at least one conventional inert excipient (or carrier), such as sodium citrate or dicalcium phosphate, or with the following components: (a) fillers or volume enhancers, such as starch, lactose, sucrose, glucose, mannitol, and silicic acid; (b) Adhesives, e.g., hydroxymethylcellulose, alginate, gelatin, polyvinylpyrrolidone, sucrose, and gum arabic; (c) humectants, e.g., glycerin; (d) Disintegrating agents, such as agar, calcium carbonate, potato starch or tapioca starch, alginic acid, certain complex silicates, and sodium carbonate; (e) Slow-solvents, such as paraffin; (f) Absorption accelerators, e.g., quaternary amine compounds; (g) Wetting agents such as cetyl alcohol and glyceryl monostearate; (h) Sorbents, e.g., kaolin; and (i) lubricants, e.g., tale, calcium stearate, magnesium stearate, solid polyethylene glycol, sodium lauryl sulfate, or a mixture thereof. In capsules, tablets and pills, the dosage form can also contain buffers.

Solid dosage forms such as tablets, sugar pills, capsules, pills, and granules can be prepared from coatings and shells, such as casings and other materials known in the field. They may contain opaques, and the release of active compounds or compounds in this composition can be released in a delayed manner in a certain part of the digestive tract. Examples of embedding components that can be used are polymeric and waxy substances. If necessary, the active compound may also be microencapsulated with one or more of the excipients mentioned above.

Liquid dosage forms for oral administration include pharmaceutically acceptable lotions, solutions, suspensions, syrups, or tinctures. In addition to active compounds, liquid dosage forms may contain inert diluents commonly used in this field, such as water or other solvents, solubilizers and emulsifiers, such as ethanol, isopropyl alcohol, ethyl carbonate, ethyl acetate, propylene glycol, 1,3-butanediol, dimethylformamide and oils, especially cottonseed oil, peanut oil, corn germ oil, olive oil, castor oil and sesame oil or a mixture of these substances.

In addition to these inert diluents, the composition may also contain additives such as wetting agents, emulsifiers and suspension agents, sweeteners, flavor correctors and fragrances.

In addition to active compounds, suspensions may contain suspension agents such as ethoxylated isooctadecanol, polyoxyethylene sorbitol and sorbita, microcrystalline cellulose, aluminum methanol and agar, or mixtures of these substances.

Compositions for parenteral injection may include physiologically acceptable sterile aqueous or anhydrous solutions, dispersions, suspensions, or emulsions, and sterile powders for resolving into sterile injectable solutions or dispersions. Suitable aqueous and non-aqueous carriers, diluents, solvents or excipients include water, ethanol, polyols and their suitable mixtures.

Dosage forms of the compound of the present invention for topical administration include ointments, powders, patches, jets, and inhalations. The active ingredient is mixed under sterile conditions with a physiologically acceptable carrier and any preservatives, buffers, or propellants that may be required if necessary.

The compound of the present invention may be administered alone or in combination with other pharmaceutically acceptable compounds.

In some embodiments, a pharmaceutical composition comprising a compound of the present invention may further include at least one additional agent. In some embodiments, at least one additional agent is selected from one or more of the checkpoint inhibitors, EGFR inhibitors, VEGF inhibitors, VEGFR inhibitors, and anticancer drugs.

In some embodiments, the drug composition described herein can include checkpoint inhibitors.

In one embodiment, the checkpoint inhibitor is an anti-PD-1 antibody, an anti-PD-L¹ antibody, or an anti-CTLA4 antibody. In one embodiment, the checkpoint inhibitor targets a checkpoint molecule that stimulates a checkpoint molecule, for example, CD27, CD40, OX⁴0, GITR, or CD138. In yet another embodiment, the checkpoint inhibitor targets to stimulate checkpoint molecules, such as A2AR, B7-H3, B7-H4, B and T lymphocyte attenuation factor (BTLA), indolamide 2,3-dioxygenase (IDO), killer cell immunoglobulin-like receptor (KIR), lymphocyte activation gene-3 (LAG3), T cell immunoglobulin and mucin domain protein 3 (TIM-3), VISTA (C10orf54) or T cell activation V-domain Ig inhibitor.

In some embodiments, the drug composition described herein includes EGFR inhibitors. In one embodiment, the EGFR inhibitors are erlotinib, gefitinib, lapatinib, panitumumab, vandetanib, or cetuximab.

In some embodiments, the drug composition described herein can include VEGF or VEGFR inhibitors. In one embodiment, the VEGF or VEGFR inhibitors are pazopanib, avitin, sorafenib, sunitinib, axitinib, punatinib, cancerreger, vandetanib, cabozantinib, ramucirumab, lenvatinib, or aflibercept.

In some embodiments, the drug composition described in this article includes anticancer drugs. Anticancer drugs can be selected from: cyclophosphamide, methotrexate, 5-fluorouracil (5-FU), doxorubicin, mustine, vincristine, methylbenzhydrazine, penicoritol, dacarbazine, bleomycin, etoposide, cisplatin, epirubicin (epirubicin). capecitabine, leucovorin, actinomycin, all-trans tretinoin, azacitidine, azathioprine, bortezomib, carboplatin, chlorambucil, cytarabine, donomycin, paclitaxel, deoxyfluorouridine, fluorouracil, gemcitabine, hydroxyurea, idarubicin, imatinib, elenodecan, nitrogen mustard (mechlorethamine)., mercaptopurine, mitoxantrone, paclitaxel, pemetrexed, teniposide, thioguanine, toploken, valrubicin (valrubicin), vinblastine, vincristine, vinpodisin, vinnopine, and oxaliplatin.

When using a drug composition, a safe and effective amount of the compound of the present invention is applied to mammals (such as humans) that require treatment, where the dose is considered effective by pharmaceutical use, and for a person weighing 60 kg, the daily dosage is usually 1~2000 mg, preferably 20-500 mg. Of course, the specific dosage should also consider factors such as the route of administration and the patient's health status, which are within the skill of a skilled physician.

### BCL-9, β-Catenin, and Wnt Signaling

Aberrant activation of Wnt signaling involves a wide variety of cancers, as tumors can rely on Wnt signaling to grow and survive. Up to 90% of all sporadic colorectal cancer cases are associated with constitutive activation of Wnt signaling.

β-catenin is a protein that can participate in protein-protein interactions, and the protein-protein interactions stimulate Wnt signal transduction, resulting in changes in transcriptional activation that may cause tumor growth and development. β-catenin is normally phosphorylated and targeted degraded by axon complexes. If stimulation of the Wnt signaling pathway is present, unphosphorylated β-catenin accumulates and binds to lymphoenhancing factor/T cytokines (LEF/TCF) and is transferred to the nucleus to stimulate transcription of Wnt target genes. The Wnt target gene contains c-myc and CD44, which are upregulated genes in tumor models. BCL9 is a protein required for efficient β-catenin-mediated transcription in mammalian cells.

“Classical” Wnt/β-catenin signaling is a pathway activated by the binding of Wnt ligands to the Frizzled family of cell surface receptors, which then regulate the expression and intracellular localization of β-catenin. In the absence of Wnt ligands, β-catenin is absent by adenomatous colonic polyposis (APC), glycogen synthase kinase-3 (GSK-3), casein kinase-1 (CK1). and axonin disruption complexes are phosphorylated and ubiquitinated and targeted for degradation in a proteasome-dependent manner. In the presence of Wnt ligands, the ubiquitination of β-catenin in the complex is inhibited, resulting in saturation of phosphorylated β-catenin, and the phosphorylated β-catenin is then stabilized and transferred to the nucleus. There, phosphorylated β-catenin is involved in nuclear T cytokine (TCF) transcription factors, such as lymphoenophonic factor/3 (LEF/TCF), to induce the expression of genes that promote cell proliferation, migration, and survival, including c-Myc and Cyclin D.

Several molecules containing BCL9 and its congener B-cell lymphoma 9-like (B9L) have been shown to be coactivators of Wnt/β-catenin transcription. The formation of a complex consisting of TCF, β-catenin, and BCL9 (or B9L) enhances β-catenin-dependent Wnt transcriptional activity. In normal cells, this transcriptional pathway is turned off when the Wnt ligand is uncoupled from its receptor. However, various loss-of-function mutations in APCs and axonins, as well as activating mutations in β-catenin itself, allow β-catenin to escape disruption of the complex and accumulate in the nucleus. Such inappropriate persistence of β-catenin has contributed to the development of a wide range of common human epithelial carcinomas, including hepatocellular carcinoma, breast cancer, colorectal cancer, and hematologic malignancies such as multiple myeloma. In addition, active β-catenin signaling leads to T cell rejection, specifically CD8+ T cell rejection, which leads to therapy resistance and reduces patient survival. Therefore, blocking Wnt signaling by targeting β-cat may provide a robust pathway for the treatment of CRC, potentially halting tumor development and metastasis.

Similar to other transcription factors, the development of selective and non-toxic β-catenin inhibitors and their clinical translation has proven to be a considerable challenge because β-catenin interacts with most of its protein partners through the same binding surface. Therefore, Wnt pathway inhibitors targeting this co-binding surface have shown significant adverse effects in animal and clinical trials. There are only a few drugs targeting β-catenin in clinical trials, including PRI-724 (Eisai Pharmaceuticals; Phase II), LGK974 (Novartis; Phase I), and OMP-54F28 and OMP-18R⁵ (OncoMed/Bayer; Phase I). In addition, disruption of LEF/TCF interactions by small molecule and β-cat peptide inhibitors can have serious side effects, including the treatment of severe bone marrow dysplasia, anemia, and general wasting in mice-which may be the result of disrupting homeostatic Wnt signaling in normal hematopoietic stem cells and intestinal stem cells. Such therapeutic limitations may arise from disruption of the interaction between β-catenin-TCF and β-catenin-E-cadherin, which may affect the integrity of epithelial tissue. In addition, biologics targeting the Frizzled receptors (OMP-54F28 and OMP-18R⁵) showed significant bone marrow toxicity during clinical trials. Wnt ligands are required for Wnt/β-cat activation, but APC and β-catenin mutations in cancer cells can induce downstream transcription without Wnt ligand activation, so blocking Wnt secretion cannot inhibit endogenous oncogenic Wnt activity caused by APC and β-catenin mutations inducing downstream gene transcription. As identified by certain biomarkers, LGK974 targets only a small subset of patients. PRI-724, a small molecule inhibitor, is being used in phase II trials utilizing daily infusions, but intravenous (IV) doses more than once a week have shown undesirable and untenable properties for clinical development.

Traditionally, the Wnt signal transduction pathway has encompassed three different types of signal transduction: the canonical Wnt signal transduction pathway, in which Wnt regulates various transcription target genes in a β-catenin-dependent manner; The non-canonical Wnt signal transduction pathway mainly involves the polarity of planar cells, in which Wnt can function independently of β-catenin; and the non-classical Wnt/calcium pathway that regulates intracellular calcium levels. In the present application, “classical Wnt signal transduction” is interchangeably referred to as “classical Wnt/β-catenin signal transduction” or “Wnt signal transduction”. As described herein, classical Wnt/β-catenin signaling can refer to a pathway component that controls the amount of β-catenin in a patient or sample by transmodulating the stability of β-catenin. In some embodiments, classical Wnt/β-catenin signaling involves transcriptional transduction of one or more genes such as c-myc, ccnd1, cd44, LGR5, VEGFA, and morePathway components of AXIN2 and LEF1. In some embodiments, classical Wnt/β-catenin signaling includes pathway components that are transduced through the interaction between β-catenin and BCL9. In some embodiments, classical Wnt/β-catenin signaling involves transcription of one or more genes controlled by the interaction between β-catenin and BCL9. One or more genes controlled by the interaction between β-catenin and BCL9 can contain c-myc, ccnd1, cd44, LGR5, VEGFA, AXIN2 and LEF1. In some embodiments, classical Wnt/β-catenin signaling includes one or more proteins, and the transcriptional expression of one or more proteins is transduced by the interaction between β-catenin and BCL9. These components can include, for example, c-Myc, Cyclin D1, CD44, LGR5, VEGFA, AXIN2, and LEF1.

How to use: In some embodiments, the compound of the present invention is applied to the object to inhibit Wnt signal transduction in the object. In some embodiments, the compound of the present invention is administered to inhibit the binding of BCL9 to β-catenin. In some embodiments, classical Wnt/β-catenin signaling of the compound of the present invention is administered. In some embodiments, the compound of the present invention is administered to treat the disease in the subject.

In some embodiments, the compound of the present invention is capable of inhibiting the binding of BCL9 to β-catenin in vitro and/or in vivo. In some embodiments, the compound of the present invention has one or more improved effects. One or more of the effects can be selected from one or more of the following: (1) inhibiting the binding of BCL9 to β-catenin; (2) inhibition of classical Wnt signal transduction; (3) reduce regulatory T cell survival; (4) Reduce the expression of VEGF in tumors; (5) increase the infiltration of CD4+ T cells and CD8+ T cells into the tumor; (6) increase T-helper 17 (Th17) cells entering the tumor; (7) Reduce intratumoral dendritic cells; (8) The half-life (T1/2) is greater than 2 hours when applied to the subject; (9) induce a tumor microenvironment conducive to immune response; and (10) inhibition of tumor growth, tumor stem cell proliferation, and/or tumor metastasis.

In some embodiments, the compound of the present invention exhibits favorable biological functions in some or each of the categories listed above, for example, in various biochemical and cellular bioassays containing potency in cell-based Wnt and/or β-catenin transcription assays.

### BCL9 Binds to β-Catenin

Pygopus (Pygo) and Legless (Lgs) are found in Drosophila as new components of Wnt signaling necessary for armadillo-mediated transcription during normal development. Pygo and BCL9/Legless transduce Wnt signaling by promoting the transcriptional activity of β-catenin/armadillo in normal and malignant cells. The ability of compounds to inhibit BCL9 binding to β-catenin can be evaluated in a variety of assays for inhibition in this field. In some embodiments, the ability of the compound of the invention to inhibit the binding of BCL9 to β-catenin can be evaluated using a homogeneous time-resolved fluorescence (HTRFRF) binding assay. In this assay, the compound/small molecule binds to a marker that recognizes another marker attached to another marker (i.e., β-catenin) attached to another marker. When a compound/small molecule binds to the target protein and therefore the two markers are in close proximity, the signal is generated and can be read quantitatively to calculate the binding affinity of the compound/small molecule. In some embodiments, the binding affinity of the compound/small molecule in this assay is compared to the binding affinity of the control to detect the improved binding affinity compared to the binding affinity of the control.

In some embodiments, the ability of the compound of the invention to inhibit the binding of BCL9 to β-catenin can be evaluated in amplified luminescence proximity homogeneous analysis (ALPHA). In this assay, the compound is conjugated to the donor bead and its target protein (i.e., β-catenin) is attached to the receptor bead. When two beads are close to each other due to the compound's binding to the target protein, a signal is generated and the binding affinity of the compound can be quantitatively calculated. In some embodiments, the binding affinity of the compounds in this assay is compared to the binding affinity of the medium or control to detect the improved binding affinity compared to the binding affinity of the medium or control.

In each embodiment, the ability of the compound of the present invention to inhibit the binding of BCL9 to β-catenin can be evaluated in the Wnt transcription assay. In some embodiments, the Wnt transcription assay is a cell-based assay. In some embodiments, the cell-based Wnt transcription assay is a β-lactamase (BLA) reporter assay. Various cell lines, transformed cell lines, or primary cells derived from healthy or diseased subjects can be used for this assay. Cell lines known to rely on classical Wnt/β-catenin signaling for their survival can also be used. In some embodiments, CellSensor™ LEF/TCF-bla HCT-116 cells and Cignal Wnt reporter are used for this reporter assay. These cells contain β-lactamase (BLA) reporter genes under the control of β-lactamase/LEF/TCF response elements stably integrated into HCT-116 cells. Due to the constitutive expression of β-lactamase by cells, the inclusion of a compound that inhibits BCL9 binding to β-catenin in this assay reduces β-lactamase production. Therefore, the potency of compounds in inhibiting Wnt transcription can be quantitatively calculated in this assay.

In some embodiments, the ability of the compound of the invention to inhibit the binding of BLC9 to β-catenin can be evaluated in cell viability assays. In some embodiments, the cell viability assay is the CellTiterGlo luminescence assay, in which cell viability is quantitatively measured. Various cell lines, transformed cell lines, or primary cells derived from healthy or diseased subjects can be used for this assay.

### Classical Wnt/β-Catenin Signaling

In some embodiments, the ability of the compound of the present invention to inhibit classical Wnt/β-catenin signal transduction can be evaluated in various in vitro and/or bulk assays. In some embodiments, the effect of the compound of the present invention on classical Wnt/β-catenin signaling is evaluated in a cell-based Wnt transcription assay, such as the β-lactamase (bla) reporter assay. The β-lactamase (BLA) reporter assay measures the strength of classical Wnt/β-catenin signaling through its ability to control β-catenin/LEF/TCF response elements and can therefore be used to assess whether the test agent can attenuate or increase classical Wnt/β-catenin signal transcription controls its transcriptional targeting.

The ability of the compound of the present invention to inhibit classical Wnt/β-catenin signaling can also be assessed by measuring gene expression and/or protein expression of target genes controlled by classical Wnt/β-catenin signal transduction transcription. The expression of target genes can be assessed in transcriptional cells in contact with compounds of the present invention or in subjects administered with these compounds. Target genes include, for example, CMYC, CCND1, CD44, LGR5, VEGFA, AXIN2, and LEF1. The expression levels of one or more target genes associated with classical Wnt/β-catenin signaling can be analyzed using methods known in this field, such as cell staining, flow cytometry, western blotting, and/or real-time quantitative PCR (rt-qPCR) analysis.

### Regulatory T Cells Survive

Various markers such as CD4, FOXP3, and CD25 are known to be expressed on regulatory T cells. The ability of the compound of the present invention to reduce the survival of regulatory T cells can be assessed by counting the total number of regulatory T cells present in the blood and/or specific tissues (such as tumors). For example, samples obtained from objects in contact with compounds of the invention can be stained with antibodies that detect markers associated with regulatory T cells. Samples can also be processed and labeled with antibodies that detect such markers and analyzed by flow cytometry. Gene and/or protein expression of such markers can be determined in samples and analyzed by, for example, Western blotting and/or rt-qPCR.

### VEGF Expression in Tumors

Various assays can be used to measure the gene expression and/or protein expression of VEGF in tumor samples. For example, after exposing the subject to the compound, tumor cells can be collected and stained with anti-VEGF antibodies to detect VEGF proteins. Cells can also be analyzed by, for example, rt-qPCR to determine gene expression of VEGF. Other assays that indicate changes in VEGF expression may be used. For example, tumor samples from subjects in contact with the compound of the invention can be analyzed to detect various generative vascular markers controlled by VEGF. In some embodiments, the compound of the present invention is more effective at reducing the expression of VEGF than the medium or control.

CD4+ and/or CD8+ T Cells Infiltrate into the Tumor

The infiltration of CD4+ T cells and/or CD8+ T cells into the tumor can be assessed by counting the total number of CD4+ T cells and/or CD8+ T cells present in or from the tumor sample (e.g., biopsy). Various markers are known to be expressed on CD4+ T cells, also known as helper T cells, for example, CD4 and CD45. Various markers are known to be expressed on CD8+ T cells (also known as cytotoxic T cells), e.g., CD8 and CD45. The ability of compounds to increase the infiltration of CD4+ and/or CD8+ T cells into tumors can be assessed in vivo by administering the compound to subjects with tumors. Tumor samples can be collected from subjects and stained with antibodies that detect markers associated with CD4+/CD8+ T cells. Samples can also be processed and labeled with, for example, antibody labels for the detection of such markers, and analyzed, for example, by flow cytometry. Gene and/or protein expression of such markers can also be determined in samples and analyzed, for example, by Western blotting and/or rt-qPCR.

### T-Assisted 17 Cells Infiltrate the Tumor

In some embodiments, the compound of the invention is able to increase the infiltration of T-helped 17 cells into the tumor when administered to a tumor-carrying subject. The entry of T-helper 17 cells into the tumor can be assessed by counting the total number of T helper 17 cells present in the tumor. Various markers are known to be expressed on T-helper 17 cells, e.g., IL-17. The ability of compounds to increase the infiltration of T-assisted 17 cells into tumors can be assessed by administering the compound in vivo to subjects with tumors. Tumor samples can be collected from the subject and stained, for example, with antibodies that detect markers associated with T-helper 17 cells. Samples can also be processed and labeled with antibodies that detect such markers and analyzed by flow cytometry. Gene and/or protein expression of such markers can also be determined in samples and analyzed, for example, by Western blotting and/or rt-qPCR. Samples can be analyzed to detect the amount of IL-17 present in the sample.

### Dendritic Cells in Tumors

In some embodiments, the compound of the invention is capable of transducing dendritic cells present in tumors when administered to tumor-bearing subjects. The number of dendritic cells present in the tumor can be assessed, for example, by staining the tumor with antibodies that recognize one or more markers associated with dendritic cells. Various markers are known to be expressed on dendritic cells, e.g., CD11c. The ability of compounds to reduce dendritic cells in tumors can be assessed in vivo by administering the compound to the subject. Tumor samples can be collected from the subject and stained with antibodies that detect markers associated with dendritic cells. Samples can also be processed and labeled, for example, with antibodies that detect such markers, and analyzed, for example, by flow cytometry. Gene and/or protein expression of such markers is analyzed by, for example, Western blotting and/or rt-qPCR.

### Biomarkers

This disclosure also covers methods for measuring at least one biomarker used to monitor the therapeutic efficacy of a compound or drug composition of the invention or for the selection of subjects to be treated with such compounds or drug compositions. In some embodiments, the biomarkers are BCL9, CD44, Axin2, cMyc, LGR5, VEGFA, Sox2, Oct4, Nanog, and/or one or more of the active β-catenins. As used herein, active β-catenin refers to the non-phosphorylated form of β-catenin.

Various known methods can be used to measure gene expression levels and/or protein levels for such biomarkers. For example, samples from subjects treated with compounds or drug compositions, such as tumors, blood, plasma, serum, urine, amniotic fluid, synovial fluid, endothelial cells, white blood cells, monocytes, other cells, organs, tissues, bone marrow, lymph nodes, or spleen, can be obtained. In some embodiments, the sample is a tumor biopsy in the subject. Samples obtained from subjects can be stained with one or more antibodies or other detection reagents that detect such biomarkers. Samples can also or alternatively be processed to detect the presence of nucleic acids encoding biomarkers (such as mRNA) by, for example, rt-qPCR methods.

In some embodiments, BCL9, CD44, Axin2, cMyc, LGR5, VEGFA, Reduced gene expression levels and/or protein levels of Sox2, Oct4, Nanog, and/or active β-catenin indicate the therapeutic potency of the compounds or drug compositions described herein. The expression levels of such biomarkers can be measured, for example, after 1 day, 2 days, 3 days, 4 days, 5 days, one week, or two weeks after administration of a compound or drug composition, or any time period in between. In some embodiments, a method is disclosed which consists of measuring the level of one or more biomarkers after one or more rounds of use of the compound or drug composition of the invention. In some embodiments, the described method further includes the continued administration of a compound or drug composition if the biomarker level decreases. In some embodiments, the described method further includes administering an increasing dose of the invention compound or drug composition if the biomarker level is not reduced, or increasing the frequency of subsequent administration. In some embodiments, treatment is discontinued if biomarker levels do not decrease after initial application. In each embodiment, the marker level is also measured before the first use of the compound or drug composition of the invention and compared with the level after one or more rounds of administration, where the therapeutic efficacy and continued treatment steps are determined based on the change in the level of one biomarker compared to one or more levels prior to administration.

In some embodiments, BCL9, CD44, Axin2, cMyc, LGR5, VEGFA, Increased gene expression levels and/or protein levels of Sox2, Oct4, Nanog, and/or active β-catenin indicate a higher level of gene expression than no increased gene expression levels and/or proteins or protein level subjects who will benefit from treatment with compounds or drug compositions of the invention. In some embodiments, treatments are disclosed, which include the selection of patients with increased levels of biomarkers and the administration of compounds or drug compositions of the invention.

In some embodiments, BCL9, CD44, Axin2, cMyc, LGR5, VEGFA, Sox2, Subjects with elevated levels of expression of the genes and/or proteins of Oct4, Nanog, and/or active β-catenin for treatment with compounds or drug compositions of the present invention. In some embodiments, tumor samples are obtained from subjects and identified with BCL9, CD44, Axin2, cMyc, LGR5, VEGFA, Tumor subjects are selected for treatment after expression of elevated genes and/or proteins in Sox2, Oct4, Nanog, and/or active β-catenin. In some embodiments, subjects with tumors are selected for treatment after obtaining tumor samples from subjects and identifying BCL9-elevated gene and/or protein expression. In some embodiments, subjects with tumors are selected for treatment after obtaining tumor samples from subjects and identifying CD44-elevated gene and/or protein expression. In some embodiments, subjects with tumors are selected for treatment after obtaining a tumor sample from the subject and identifying the expression of the gene and/or protein with elevated active β-catenin.

### Half-Life in the Receptor

In some embodiments, the compound of the invention has one or more improved pharmacokinetic parameters compared to the medium or control. Such pharmacokinetic parameters can include, for example, maximum observed concentration (Cmax), time to maximum concentration (Tmax), final half-life (T1/2), systemic clearance (CL), volume of distribution (Vz)., area under the curve from application time to the last measurable concentration (AUC0-t), area under the curve extrapolated from application time to infinity (AUC0-inf), and bioavailability.

Methods used to evaluate the pharmacokinetics of agents are known in this field. For example, it can be 5 minutes after administration, 1, 2, 4, 6, 8, Obtain blood samples from subjects administered at 12 and 24 h for the compounds described herein. The concentration of compounds in blood samples can be analyzed by various analytical tools, such as LC/MS. Pharmacokinetic parameters are calculated based on compound concentrations at each time point. As used herein, the term “maximum observed concentration (Cmax)” refers to the maximum serum concentration achieved by the compound after application. Related to the concept of Cmax, the time to maximum concentration (Tmax) is the time when a compound reaches its maximum serum concentration. The terms “terminal half-life (T1/2)” and “half-life (T1/2)” are used interchangeably and refer to the time at which a compound loses half of its serum concentration. Systemic clearance (CL) indicates the amount of blood that completely clears a compound per unit of time. The term “volume of distribution (Vz)” refers to the theoretically calculated volume that needs to contain the total amount of compound administered to the subject at the same concentration observed in the blood. The term “bioavailability” refers to the extent and rate at which a drug is absorbed into a biological system or is available at a physiologically active site. Bioavailability can be a function of several of the properties previously described, including stability, solubility, immunogenicity, and pharmacokinetics, and can be assessed using methods known to those skilled in the field.

It can be found in mammals (including, for example, mice, rats, or humans). Pharmacokinetic parameters of the compound are evaluated. Parameters can also be assessed using various routes of administration, such as intravenous, intraperitoneal, subcutaneous, and intramuscular routes. In some embodiments, the pharmacokinetic parameters of the compound of the present invention are evaluated in mice. In some embodiments, the pharmacokinetic parameters of the compounds described here are evaluated in mice administered subcutaneously. In some embodiments, the pharmacokinetic parameters of the compound of the present invention are evaluated in the human sacrum. In some embodiments, the pharmacokinetic parameters of the compound of the invention are evaluated in humans after subcutaneous administration.

### Tumor Microenvironment Conducive to Immune Response

In each embodiment, the compound of the present invention induces a tumor microenvironment conducive to an immune response. In each embodiment, the compound of the present invention induces a tumor microenvironment that is more conducive to the immune response than the medium or control.

Various parameters can be used to assess the tumor microenvironment. For example, an increased ratio between cytotoxic T cells in and/or around tumor tissue and regulatory T cells can indicate that the tumor microenvironment favors the immune response. A decrease in the number of dendritic cells and/or regulatory T cells in and/or around tumor tissue may also indicate a favorable tumor microenvironment for the immune response. Other parameters include an increase in circulating T cells in peripheral blood and an increase in the ratio between T helper 17 cells in and/or surrounding tumor tissue and regulatory T cells. These parameters can indicate that the tumor microenvironment is conducive to the immune response.

In some embodiments, the compound of the present invention can increase the ratio of the amount of cytotoxic T cells to the amount of regulatory T cells in the tumor microenvironment. In some embodiments, the rate change caused by the compound is greater than the rate change caused by the medium or control.

Tumor Growth, Tumor Stem Cell Proliferation, and/or Tumor Metastasis

Since Wnt signaling is a modulator of tumor growth, the therapeutic potency of compounds affecting BCL9 binding to β-catenin can be evaluated in animal models.

In vivo potency of the invention can be evaluated in human cancer models using e.g., BALB/c nude mice, as xenografts of human cancer cells will grow into tumors in these mice. For example, Colo320DM tumor cells, a commercially available cell line derived from human colon cancer tissue, can be used to form tumors in BALB/c nude mice. Additional in vivo models can also be used to evaluate the in vivo potency of the compounds published herein. For example, human DLD-1 colon cancer cells can be implanted into nude mice to assess tumor growth. A CT26 isogeneic mouse model of colon cancer can also be used, as the described model allows for the assessment of tumor growth in the context of an intact immune system. Other types of cancer cells, such as B16 melanoma, 4T1 breast cancer, human kidney cancer, and Lewis lung cancer cells, can also be used in these known animal models to evaluate the in vivo potency of the compounds published herein.

By administering the compound of the invention to one or more animal models, the role of the compound in reducing tumor growth in vivo can be evaluated. Based on animal data treated with stable BCL9 peptides, the ability of peptides to inhibit Wnt signaling can be assessed by, for example, staining tissue samples with markers of Wnt signaling. These downstream markers of Wnt signal transduction include, for example, Axin2 and CD44.

In situ mouse models can be used to evaluate the effects of the compounds described herein on tumor metastasis. For example, orthotropic animal models can be injected with cells carrying luciferase constructs and then administered with their assigned treatment. The presence of injected cells can be detected by administering fluorescein substrate to each treated animal. The intensity of the bioluminescence signal can be quantitatively measured and used as an indicator of cell growth.

In some embodiments, the effect of the compound of the present invention on cancer stem cell proliferation can be assessed by measuring biomarkers of various cancer stem cells. For example, the expression level of CD44 and/or LGR5 can indicate the amount of cancer stem cells present in the sample. Tumor samples can be collected from the subject and stained with antibodies that detect markers associated with cancer stem cells. Samples can also be processed and labeled, for example, with antibodies that detect such markers, and analyzed, for example, by flow cytometry. Gene and/or protein expression of such markers can be detected and analyzed, for example, by Western blotting and/or rt-qPCR.

Diseases with Abnormal Wnt/β-Catenin Signaling

Abnormal Wnt/β-catenin signaling is associated with malignant transformation of normal cells into cancer cells. Activation of Wnt signaling and nuclear localization of β-catenin is associated with tumor phenotypes in multiple models.

This disclosure covers the composition used and the method of using the compound disclosed herein to inhibit the binding of BCL9 to β-catenin in the subject by administering a compound to the subject or a drug composition containing the compound. This publication also covers inhibition of classical Wnt/β-catenin signaling in subjects by administering compounds or drug compositions published herein. This disclosure further covers methods for treating the subject's disease by administering the compound or drug composition of the invention to the subject. The disease may be a cancer or other neoplastic disease associated with abnormal classical Wnt/β-catenin signaling.

In some embodiments, a disease, condition, or condition can be a disease that benefits from inhibition of typical Wnt/β-catenin signaling. In some embodiments, such diseases, conditions, or medical conditions are cancer. In some embodiments, the cancer is a cancer with high expression of BCL9 and/or β-catenin. In some embodiments, cancer is cancer co-localized by BCL9 and β-catenin in the nucleus of cancer cells. In some embodiments, the cancers are selected from: familial adenomatous polyposis (FAP), eye cancer, rectal cancer, colon cancer, colorectal cancer, cervical cancer, prostate cancer, breast cancer, bladder cancer, oral cancer, benign and malignant tumors, stomach cancer, liver cancer, pancreatic cancer, lung cancer, uterine body, ovarian cancer, prostate cancer, testicular cancer, kidney cancer, brain cancer/CNS Cancer, Laryngeal Cancer, Multiple Myeloma, Cutaneous Melanoma, Acute Lymphoblastic Leukemia, Acute Myeloid Leukemia, Ewing's Sarcoma, Kaposi's Sarcoma, Basal Cell Carcinoma and Squamous Cell Carcinoma, Small Cell Lung Cancer, Chorionic Carcinoma, Rhabdomyosarcoma, Hemangiosarcoma, Hemangiosarcoma, Hemangioendothelioma, Wilm's Tumor, Neuroblastoma, Oral/Pharyngeal Cancer, Esophageal Cancer, Laryngeal Cancer, Lymphoma, Neurofibromatosis, Tuberous Sclerosis, Hemangioma, Gastric Cancer (gastric cancer), ovarian cancer, hepatocellular carcinoma, and lymphangiogenesis. In some embodiments, said cancer is colorectal cancer. In some implementations, said cancer is stomach cancer. In some embodiments, said cancer is ovarian cancer. In some embodiments, said cancer is hepatocellular carcinoma. In some embodiments, said cancer is breast cancer. In some embodiments, the cancer in question is prostate cancer. In some embodiments, said cancer is cutaneous melanoma. In some implementations, said cancer is lung cancer.

In some embodiments, any compound or variant of the compound or variant published herein or a drug composition that includes such compounds may be used to treat diseases, such as cancer listed above.

Treatment and measured therapeutic parameters can be evaluated after administration alone or in combination with one or more additional therapeutic agents (e.g., as a single bolus or separate sequential administration) of a compound or drug composition. The additional agent may be any additional therapeutic agent mentioned herein or known to a person skilled in the field. Depending on the chosen protocol, the compound or drug composition including the compound and/or additional agents can be administered once or more times.

The present invention also covers compounds or drug compositions disclosed herein for the treatment of diseases in the object. In some embodiments, the disease can benefit from inhibition of the canonical Wnt/β-catenin signaling. In some implementations, the disease is cancer.

This disclosure further covers the use of compounds or drug compositions disclosed herein in the manufacture of drugs for the treatment of diseases in the subject. In some embodiments, the disease can benefit from inhibition of the canonical Wnt/β-catenin signaling. In some implementations, the disease is cancer.

In another embodiment, the disease being treated is a disease other than cancer. In some embodiments, the diseases are bone density defects, eye vascular defects, familial exudative vitreoretinopathy, early coronary heart disease, Alzheimer's disease, autosomal dominant oligodontism, retinal angiogenesis, osteogenesis imperfecta, Tetra-Amelia syndrome, Mullerian duct degeneration and virilization regression andvirilization), SERKAL syndrome, type II diabetes mellitus, Fuhrmann syndrome, nail dermal dysplasia, obesity, cleft hand and foot deformity, caudal duplication, dental hypoplasia, skeletal dysplasia, local dermal hypoplasia, autosomal recessive scleroderma, Neural tube defects or sclerosing ossification and Van Buchem disease.

### Combination Therapy

In some embodiments, the compounds or drug compositions disclosed herein are administered with at least one additional agent. This means that the compound and additional agents can be administered to patients continuously or simultaneously in separate dosage forms as described herein. In some embodiments, at least one additional agent is selected from a checkpoint inhibitor, an EGFR inhibitor, a VEGF inhibitor, a VEGFR inhibitor, an anticancer drug (e.g., any therapeutic agent in the additional therapeutic agent described herein), a peptide, and an additional agent may be administered at therapeutically effective doses.

In some embodiments, subjects administering compounds or drug compositions disclosed herein are treated with radiation therapy and/or chemotherapy before, after, or at the same time as the administration of the compound or drug composition.

### Kits

The present invention also comprises a drug kit that can be used for, for example, the treatment of the conditions, diseases and conditions described herein, and the drug kit comprises one or more containers containing a drug composition, and the drug composition includes a compound of the present invention for the treatment of an effective amount. If desired, such kits can also contain a variety of conventional drug kit components, e.g., one or more of containers with one or more pharmaceutically acceptable carriers, additional containers, etc. The kit may also include an insert or as a label instruction sheet stating the amount of component to be administered, application guidelines, and/or guidelines for mixing components.

The kit used to perform the method described in this article is also exposed. In each embodiment, a kit for the manufacture of the compound of the present invention is provided. In some embodiments, the kit includes compounds capable of undergoing reactions used to form one or more hydrocarbon linking groups. In some embodiments, the kit includes a metal catalyst for performing metal-mediated shut-off ring metathesis.

In some embodiments, the kit includes a test for BCL9, CD44, Axin2, cMyc, LGR′, VEGFA, Sox2, Oct4, Nanog, and/or agents that express genes and/or proteins that are active β-catenin.

The present invention is further elaborated in combination with specific embodiments. It should be understood that these embodiments are used only to illustrate the present invention and not to limit the scope of the present invention. The experimental methods in which specific conditions are not specified in the following embodiments are usually in accordance with the conventional conditions or according to the conditions recommended by the manufacturer. Unless otherwise noted, the percentage and number of servings are the percentage by weight and the number of copies by weight.

### Preparation Examples

### Universal Synthesis Method

The compound of the present invention may be prepared, isolated, or obtained by any method obvious to a person skilled in the field. The compound of the present invention may also be prepared according to the exemplary preparation scheme provided below, such as the method in the embodiment. The reaction conditions, steps, and reactants not provided in the exemplary preparation protocol are obvious and known to those skilled in the field. As used herein, the symbols and conventions used in these processes, schemes and embodiments, whether or not specific abbreviations are specifically defined, have meanings well known to people skilled in the field.

Specifically, but not limited to, the following abbreviations may be used in embodiments and throughout the specification: R.T. (room temperature); g (g); mg (mg); mL (milliliters); L (microliters); millimeters (mmol); M (micromolars); MHz (Hertz); MHz (megahertz); mmol (millimoles); hr (hours); min; MS) (mass spectrometry); ESI (Electrospray Ionization); TLC (thin layer chromatography); HPLC (High Performance Liquid Chromatography); BOC (tert-butoxycarbonyl); tBu (tert-butyl); HATU (2-(7-azabenzotriazole)-N,N,N′,N′-tetramethylurea hexafluorophosphate); TFA (trifluoroacetic acid); PD2(DBA)₃ (tris(diabenzyl acetone)dipalladium); DIPEA (N,N-diisopropylethylamine).

### Preparation Example 1: Synthesis of Compounds 115-010, 115-011, 115-012, 115-013, 115-014, 115-015, 115-022, 115-023

### (1) Synthesis of Compound 115-010

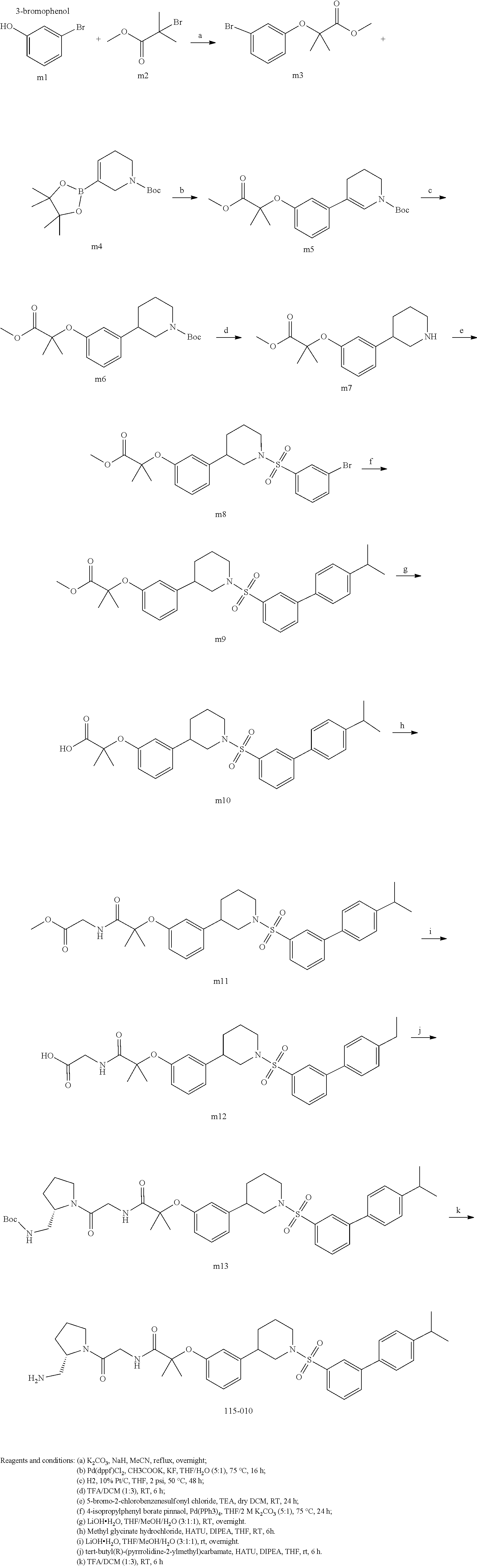

### Specific Steps

Synthesis of intermediate m3: raw materials m1 (2.00 g, 1 eq), m2 (6.28 g, 3 eq), K₂CO₃ (6.30 g, 4 eq), and catalyst NaH (0.04 g, 0.1 eq).) to MeCN (60 mL), heat to 85 °C reflux, stir the reaction overnight. TLC is monitored until the reaction is completed, and the concentrated filtrate is evaporated under reduced pressure. The resulting residue was extracted with water and ethyl acetate, and the organic layer was washed with saturated NaCl aqueous solution, and then dried with anhydrous Na₂SO₄, and the crude product was obtained after steaming. The crude product was purified by silica column chromatography (petroleum ether/ethyl acetate=30:1), and the intermediate product m3 was obtained as a colorless oil, 2.83 g, with a yield of 89.56%.

Synthesis of intermediate m5: The raw materials m3 (0.94 g, leg), m4 (0.78 g, 1.1 eq), potassium acetate (1.35 g, 4 eq), potassium fluoride (0.60 g, 3 eq) catalyst Pd (dppf) were combined C₁₂ (0.24 g, 0.1 eq) was added to THF/H₂O (48 mL) and heated to 75 °C for 16 h under the protection of nitrogen. TLC is monitored until the reaction is completed, and the concentrated filtrate is evaporated under reduced pressure, and the crude product is obtained after evaporation. The crude product was purified by silica column chromatography (petroleum ether/ethyl acetate=6:1), and the intermediate product m5 was a light yellow oily substance, 0.88 g, with a yield of 90.7%.

Synthesis of the intermediate m6: 20% Pt/C (6 g), m5 (0.88 g, leg) was added to THF (15 mL) solution, heated to 50 °C and stirred under 2 psi hydrogen (hydrogen balloon) for 48 hours. After HPLC monitoring until the reaction is 80% complete, the insoluble substances are filtered out, and the concentrated filtrate is evaporated under reduced pressure. The intermediate product M6 (crude product) is a light yellow oil, 1.04 g, with a content of about 75% (0.78 g).

Synthesis of intermediate m7: m6 (crude product) is added to DCM (15 mL) solution, then 5 mL TFA is added to the reaction solution, stirred at room temperature for 6 h, TLC monitored until the reaction is completed, evaporated and concentrated filtrate under reduced pressure, and the crude product m7 (crude product) is obtained as a light yellow oily substance after evaporation and drying. 1.47 g, about 40% content.

Synthesis of intermediate m8: Intermediate products m7 (0.57 g, 1 eq), 5-bromo-2-chlorobenzenesulfonyl chloride (0.65 g, 1.1 eq), TEA (0.62 g, 3 eq), anhydrous DCM solution (15 ml) was added to a 50 ml round-bottom flask and stirred at room temperature under nitrogen protection. TLC monitoring until the completion of the reaction, evaporation of the concentrated filtrate under reduced pressure, dilution with water, and extraction of ethyl acetate. The organic layer was dried with anhydrous Na₂SO₄ solids, and the crude product was obtained by evaporating and concentrating the filtrate under reduced pressure. The crude product was purified by silica column chromatography (petroleum ether/ethyl acetate=5:1), and the intermediate product m8 was a colorless oil with a yield of 0.77 g and a yield of 70.64%.

Synthesis of the intermediate m9: m8 (0.30 g, 1 eq), pinnaol 4-isopropyl phenylborate (0.15 g, 1.1 eq) and Pd(PPh3)4 (21609 mg, 0.19 mmol) Add to THF/2M K₂CO₃ (5:1) (15:3 mL) solution (75 °C) and stir the reaction under nitrogen protection for 12 h. TLC monitoring until the reaction is complete, decompression evaporation of the concentrated filtrate, and the resulting residue is added with water and ethyl acetate for extraction. The organic layer was washed with saturated NaCl solution, then dried with anhydrous Na₂SO₄ solids, and the filtrate was evaporated and concentrated under reduced pressure to obtain a crude product.

The crude product was purified by silica gel column chromatography (petroleum ether/ethyl acetate=10:1), and the intermediate product m9 was obtained as a colorless oil with a yield of 74.19% of 0.23 g.

Synthesis of intermediate m10: m9 (0.23 g, leg), LiOH H₂O (69 mg, 4 eq) was added to a solution of THF/MeOH/H₂O (3:1:1) (12:4:4 mL) and stirred overnight at room temperature. TLC is monitored until the reaction is complete, and the mixture is depressurized and concentrated. The resulting residue was dissolved in ethyl acetate, adjusted to pH=2 with 1 M HCl, and then extracted with ethyl acetate. The organic layer was dried with anhydrous Na₂SO₄ solid, and the reaction liquid was evaporated and concentrated under reduced pressure, and the crude product m10 was white solid, 0.26 g, which was directly used for the next reaction.

Synthesis of intermediate m11: Add intermediate m10 (0.26 g, 0.57 mmol) to 100 ml dry reaction flask, dissolve with 40 ml THF, and add methyl glycinate hydrochloride (0.12 g, 0.93 mmol).), DIPEA (0.40 g, 3.09 mmol), HATU (0.32 g, 0.85 mmol) at room temperature for 6 h. TLC plate (petroleum ether:ethyl acetate=1:1) monitored the reaction, 30 ml of water was added to the reaction solution, DCM (30 ml×3) was extracted and separated, the organic phase was collected, washed with 40 ml saturated saline, dried with anhydrous Na₂SO₄, and the residue was chromatized by silica gel column[V (petroleum ether): V (ethyl acetate)=1: 1 as eluent] purified yellowish viscous solid product m11 (0.37 g, yield 81%).

Synthesis of the intermediate m12: Add the compound m11 (0.37 g, 0.63 mmol) in a 50 ml dry reaction flask, dissolve with 20 ml THF, and add lithium hydroxide (0.12 g, 2.52 mmol). Dissolve in 5 ml of water, then add 5 ml of methanol and mix well, and finally add it to the reaction solution and stir the reaction at room temperature for 4 h. TLC plate (petroleum ether: ethyl acetate=1:1) to monitor the reaction, concentrate the reaction solution, add 20 ml of water to the residual solution, adjust the PH of about 4-5 with 1 M HCl, extract with ethyl acetate (30 ml×3), merge with organic phases, and wash with 40 ml saturated saline, The yellowish solid compound m12 (0.33 g, yield 99%) was obtained by drying the anhydrous Na₂SO₄ three times to concentrate the organic phase.

Synthesis of intermediate m13: Add intermediate m12 (0.32 g, 0.57 mmol) to 100 ml dry reaction flask, dissolve with 15 ml THF, and add tert-butyl(R)-(pyrrolidine-2-ylmethyl group Carbamate (0.14 g, 0.69 mmol), DIPEA (0.30 g, 2.30 mmol HATU (0.24 g, 0.63 mmol) stirred at room temperature for 6 h. TLC plate (ethyl acetate) monitored the reaction, concentrated the reaction solution, added 20 ml of water to the residual solution, extracted with DCM (30 ml×3), merged with organic phases, washed with 40 ml saturated saline, dried with anhydrous Na₂SO₄, and the residue was chromatized by silica gel column [ethyl acetate is the eluent The yellowish solid product m13 (0.32 g, yield 74%) was purified. 50 Synthesis of 115-010: m13 (100 mg) was added to DCM (15 mL) solution, then 5 mL TFA was added to the reaction solution, stirred at room temperature for 6 h, TLC was monitored until the reaction was completed, the concentrated filtrate was evaporated under reduced pressure, and the crude product was obtained after evaporation, water was added, and DCM was extracted. The organic layer was washed with saturated NaCl aqueous solution, then dried with anhydrous Na₂SO₄ solids, and the reaction solution was 55 evaporated and concentrated under reduced pressure to obtain a crude product. The crude product was purified by reversed-phase preparation (water/acetonitrile/trifluoroacetic acid) to obtain product 115-010 preparation solution, and frozen in the refrigerator at −80 °C for 6 h Then the white solid end product was 115-010, 46 mg, and the yield was 54.14%. HPLC >95%.

### (2) Compounds 115-011, 115-012, 115-013, 115-014, 115-015, 115-022, 115-023 Was Prepared by a Synthesis Method Similar to the Compound 115-010

### Preparation Example 2: Synthesis of Compounds 115-016, 115-017, 115-018, 115-019

### (1) Synthesis of Compound 115-016

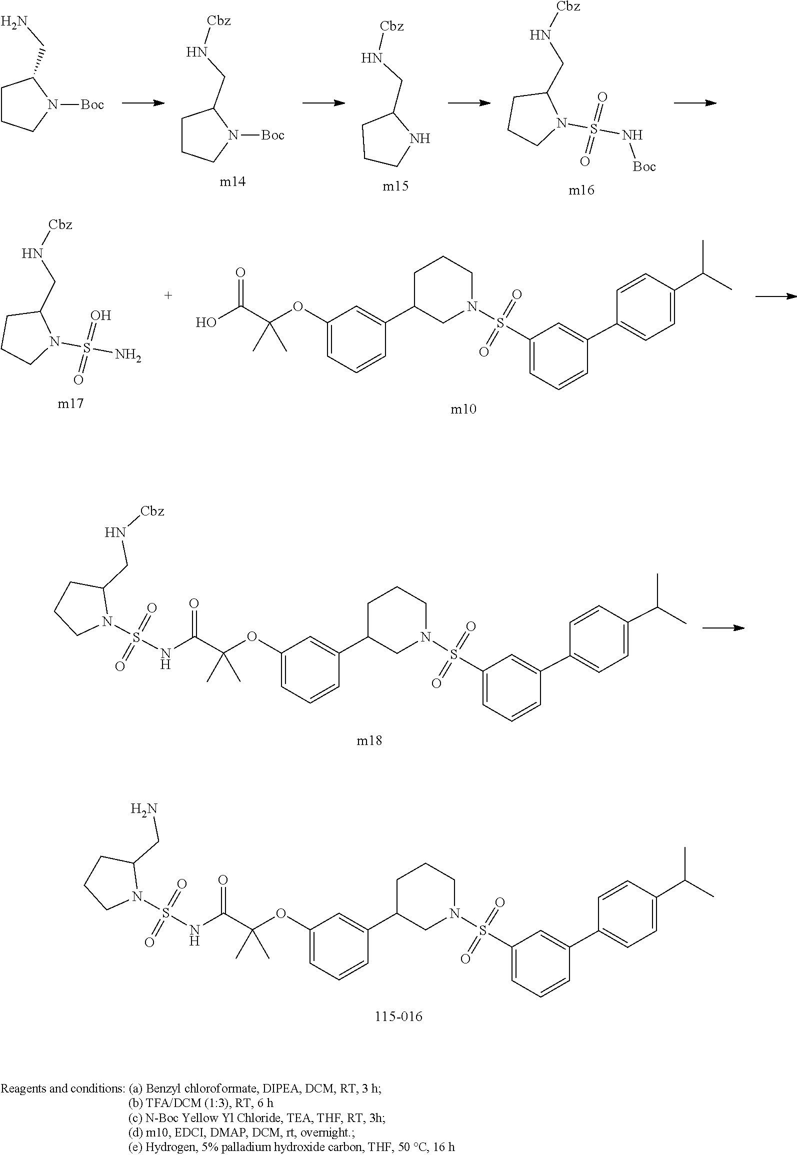

### Specific Steps

Synthesis of the intermediate m14: In a dry reaction flask, add tert-butyl(R)-2-(aminomethyl)pyrrolidin-1-carboxylate (0.5 g, 1 eq), DIPEA (1.29 g, 4 eq), DCM, then stir at room temperature for 10 minutes, then add benzyl chloroformate (0.47, 1.1 eq), stir the reaction at room temperature for 3 h, monitor with TLC until the reaction is completed, add water, and extract with DCM. The organic layer was washed with saturated NaCl aqueous solution, then dried with anhydrous Na₂SO₄ solid, and the reaction solution was evaporated and concentrated under reduced pressure to obtain the crude product, and the intermediate m14 (0.71 g) eluted by silica gel column 10:1 (petroleum ether: ethyl acetate) yield was 85.5% Synthesis of intermediate m15: In a dry reaction flask, add m14 (0.71 g, leg), 15 ml DCM, 5 ml TFA and stir at room temperature for 6 h, TLC monitoring until the reaction is completed, add water, and extract with DCM. The organic layer was washed with saturated NaCl aqueous solution, then dried with anhydrous Na₂SO₄ solid, and the crude product m15 (0.67 g) was evaporated under reduced pressure to obtain the crude product m15 (0.67 g) with a yield of 100%.

Synthesis of intermediate m16: In a dry reaction flask, add m15 (0.67 g, leg), TEA (0.84 g, 4 eq), THF to dissolve, then stir at room temperature for 30 min and then add N-Boc yellowyl chloride (1.2 eq) droplets, and continue the stirring reaction at room temperature for 6 h, TLC monitoring until the reaction is completed, evaporating and concentrating the reaction solution under reduced pressure, adding water, and extracting by DCM. The organic layer was washed with saturated NaCl aqueous solution, then dried with anhydrous Na₂SO₄ solids, and the reaction liquid was evaporated under reduced pressure to obtain a crude product, and the intermediate m16 (0.31 g) eluted by silica gel column 5:1 (petroleum ether: ethyl acetate) was obtained, with a yield of 31%.

Synthesis of intermediate m17: In a dry reaction flask, add m15 (0.31 g, leg), 15 ml DCM, 5 ml TFA, then stir at room temperature for 6 h, monitor TLC until the reaction is completed, add water, and extract with DCM. The organic layer was washed with saturated NaCl aqueous solution, then dried with anhydrous Na₂SO₄ solid, and the crude product m15 (0.38 g) yield was 100%.

Synthesis of intermediate m18: add m17 (0.14 g, 1.1 eq), m10 (0.21 g, leg), EDCI (2 eq), DMAP (2 eq), DCM in a dry reaction flask, then stir at room temperature for 6 h, monitor with TLC until the reaction is completed, add water, and extract by DCM. The organic layer was washed with saturated NaCl aqueous solution, then dried with anhydrous Na₂SO₄ solid, and the reaction liquid was evaporated and concentrated under reduced pressure to obtain the crude product, which was eluted by silica gel column 3:1 (petroleum ether: ethyl acetate) intermediate m17 (0.23 g), with a yield of 69%.

Synthesis of intermediate 115-016: In a dry reaction flask, add m18 (0.1 g, leg), 5% palladium hydroxide carbon (20% W, 0.02 g), THF, and hydrogen (2-3 Psi)., then stirred at 50 °C for 16 h, HPLC monitoring until the reaction is completed, water is added, the reaction liquid is evaporated under reduced pressure, and the crude product is extracted by DCM. The organic layer was washed with saturated NaCl aqueous solution, then dried with anhydrous Na₂SO₄ solids, and the reaction liquid was evaporated and concentrated under reduced pressure to obtain the crude product m15 (0.67 g). The crude product was purified by reversed-phase preparation (water/acetonitrile/trifluoroacetic acid) to obtain product 115-016 preparation solution, and was frozen in the refrigerator at −80 °C for 6 h Then the white solid end product was 115-016, 21 mg, and the yield was 23.14%. HPLC >95%.

### (2) Compounds 115-017, 115-018, and 115-019 were Prepared by Synthesis Method Similar to Compound 115-016

### Preparation Example 3: Synthesis of Compounds 115-020 and 115-021

### (1) Synthesis of Compound 115-020

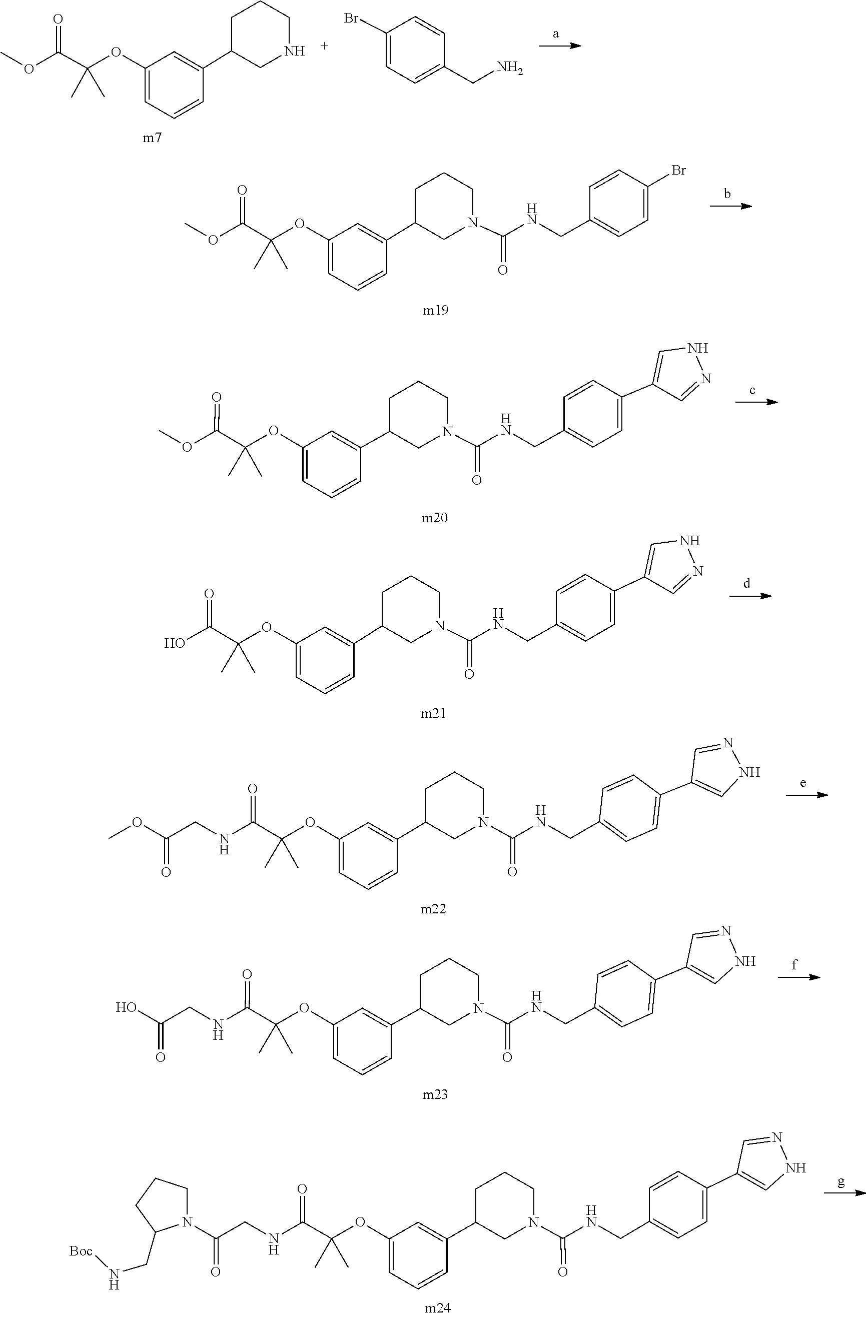

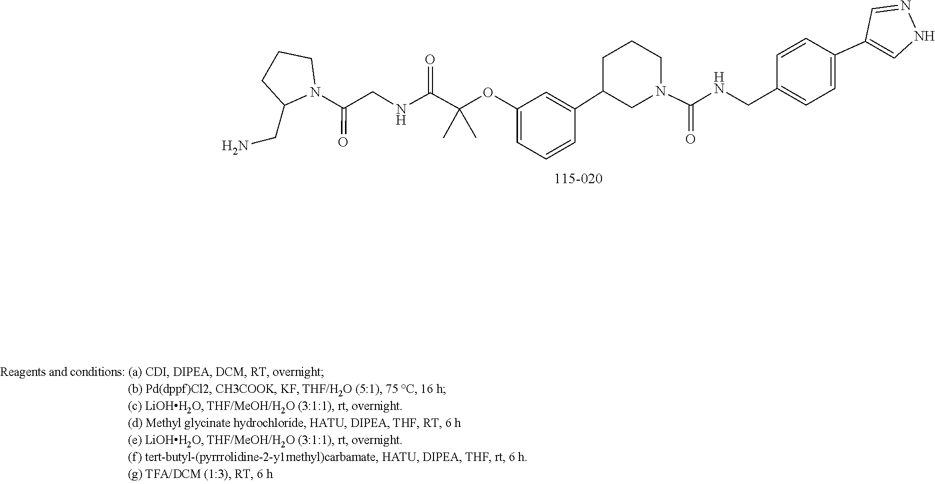 [SMILES: CC(C)(Oc1cccc(C2CCCN(C(=O)NCc3ccc(-c4cn[nH]c4)cc3)C2)c1)C(=O)NCC(=O)N1CCCC1CN]

### Specific Steps

Synthesis of the intermediate m19: 4-bromobenzylamine (0.41 g, 1 eq) was added to a 100 ml dry reaction flask, dissolved in 20 ml anhydrous DCM, then DIPEA (1.14 g, 4 eq), Stir the reaction at room temperature for 1 h, then add the intermediate m7 (0.61 g, leg) drops dissolved in 10 ml DCM to the reaction solution and continue the stirring reaction overnight at room temperature for TLC plates (petroleum ether: ethyl acetate=5:1). Monitor the response. 30 ml of water was added to the reaction solution, DCM (30 ml×3) was extracted and separated, the organic phase was collected, washed with 40 ml saturated saline, dried with anhydrous Na₂SO₄, and the residual solution was chromatized by silica gel column [V (petroleum ether): V (ethyl acetate)=5: 1 Purified intermediate m19 (0.66 g, yield 62%).

Synthesis of the intermediate m20: Add the intermediate m19 (0.66 g, 1 eq) in a 100 ml dry reaction flask, dissolve with 30 ml of dioxane, then add potassium acetate (0.53 g, 4 eq), 31 (1-Boc-pyrazole-pinnaol borate, 0.47 g, 1.2 eq), Pd(dppf)C₁₂ (0.07 g, 0.1 eq), then add 5 ml of water, stir the reaction at 80 °C under the protection of N2 for 36 h, monitor the reaction with TLC plate (petroleum ether: ethyl acetate=1:1), then cool the reaction solution to room temperature, filter to remove insoluble substances, and concentrate the filtrate The residue was purified by silica column chromatography [V (petroleum ether): V (ethyl acetate)=1:1 as eluent] to obtain the intermediate m20 (0.48 g, yield 75%).

Synthesis of the intermediate m21: Add the compound m20 (0.48 g, 1 eq) in a 50 ml dry reaction flask, dissolve with 20 ml THF, dissolve lithium hydroxide (0.17 g, 4 eq) in 5 ml of water, and add 5 ml methanol was mixed well, and finally added to the reaction solution, and the reaction was stirred at room temperature for 4 h. TLC plate (petroleum ether: ethyl acetate=1:1) monitored the reaction, concentrated the reaction solution, added 20 ml of water to the residual solution, adjusted the PH by about 4-5 with 1 M HCl, and then extracted with ethyl acetate (30 ml×3), and the organic phase was combined, washed with 40 ml saturated saline, dried with anhydrous Na₂SO₄ three times, concentrated organic phase, and obtained the yellowish solid compound m21 (0.46 g, yield 99%).

Synthesis of intermediate m22: add intermediate m21 (0.46 g, leg) in a 100 ml dry reaction flask, dissolve in 40 ml THF, and add methyl glycinate hydrochloride (0.15 g, 1.2 eq), DIPEA (0.52 g, 4 eq), HATU (0.45 g, 12 eq Room temperature stirring reaction for 6 h. TLC plate (petroleum ether:ethyl acetate=1:1) monitored the reaction, 30 ml of water was added to the reaction solution, DCM (30 ml×3) was extracted and separated, the organic phase was collected, washed with 40 ml saturated saline, dried with anhydrous Na₂SO₄, and the residue was chromatized by silica gel column[V (petroleum ether): V (ethyl acetate)=1: 1 as eluent] purified the yellowish viscous solid product m22 (0.35 g, yield 66%).

Synthesis of the intermediate M23: Add the compound m22 (0.35 g, 1 eq) in a 50 ml dry reaction flask, dissolve it with 20 ml THF, dissolve lithium hydroxide (0.11 g, 4 eq) in 5 ml water, and add 5 ml methanol was mixed well, and finally added to the reaction solution, and the reaction was stirred at room temperature for 4 h. TLC plate (petroleum ether: ethyl acetate=1:1) to monitor the reaction, concentrate the reaction solution, add 20 ml of water to the residual solution, adjust the PH of about 4-5 with 1 M HCl, extract with ethyl acetate (30 ml×3), merge with organic phases, and wash with 40 ml saturated saline, The yellowish solid compound m23 (0.34 g, yield 99%) was obtained by drying the anhydrous Na₂SO₄ three times to concentrate the organic phase.

Synthesis of intermediate m24: add intermediate m23 (0.34 g, leg) in a 100 ml dry reaction flask, 15 dissolve in 15 ml THF, and add tert-butyl-(pyrrolidine-2-ylmethyl group) Carbamate (0.14 g, 1.2 eq), DIPEA (0.40 g, 4 eq). HATU (0.27 g, 1.2 eq) was stirred at room temperature for 6 h. TLC plate (ethyl acetate) monitored the reaction, concentrated the reaction solution, added 20 ml of water to the residual solution, extracted with DCM (30 ml×3), merged with organic phases, washed with 40 ml saturated saline, dried with anhydrous Na₂SO₄, and the residue was chromatized by silica gel column [ethyl acetate is the eluent The yellowish solid product M24 (0.27 g, yield 58%) was purified.

Synthesis of 115-020: m24 (100 mg) was added to DCM (15 mL) solution, then 5 mL TFA was added to the reaction solution, stirred at room temperature for 6 h, TLC monitored until the reaction was completed, evaporated and concentrated filtrate under reduced pressure, and the crude product was obtained after evaporation, water, and DCM was extracted. The organic layer was washed with saturated NaCl aqueous solution, then dried with anhydrous Na₂SO₄ solids, and the reaction solution was evaporated and concentrated under reduced pressure to obtain a crude product. The crude product was purified by reversed-phase preparation (water/acetonitrile/trifluoroacetic acid) to obtain product 115-020 preparation solution, and was frozen in the refrigerator at −80 °C for 6 h Then the freeze-dried white solid end product was 115-020, 44 mg, with a yield of 51.76%. HPLC >95%.

### (2) Compound 115-021 was Prepared by a Synthesis Method Similar to Compound 115-020

### Preparation Example 4: Synthesis of Compounds 115-027, 115-028, 115-029, 115-030, 115-031

### (1) Synthesis of Compound 115-027

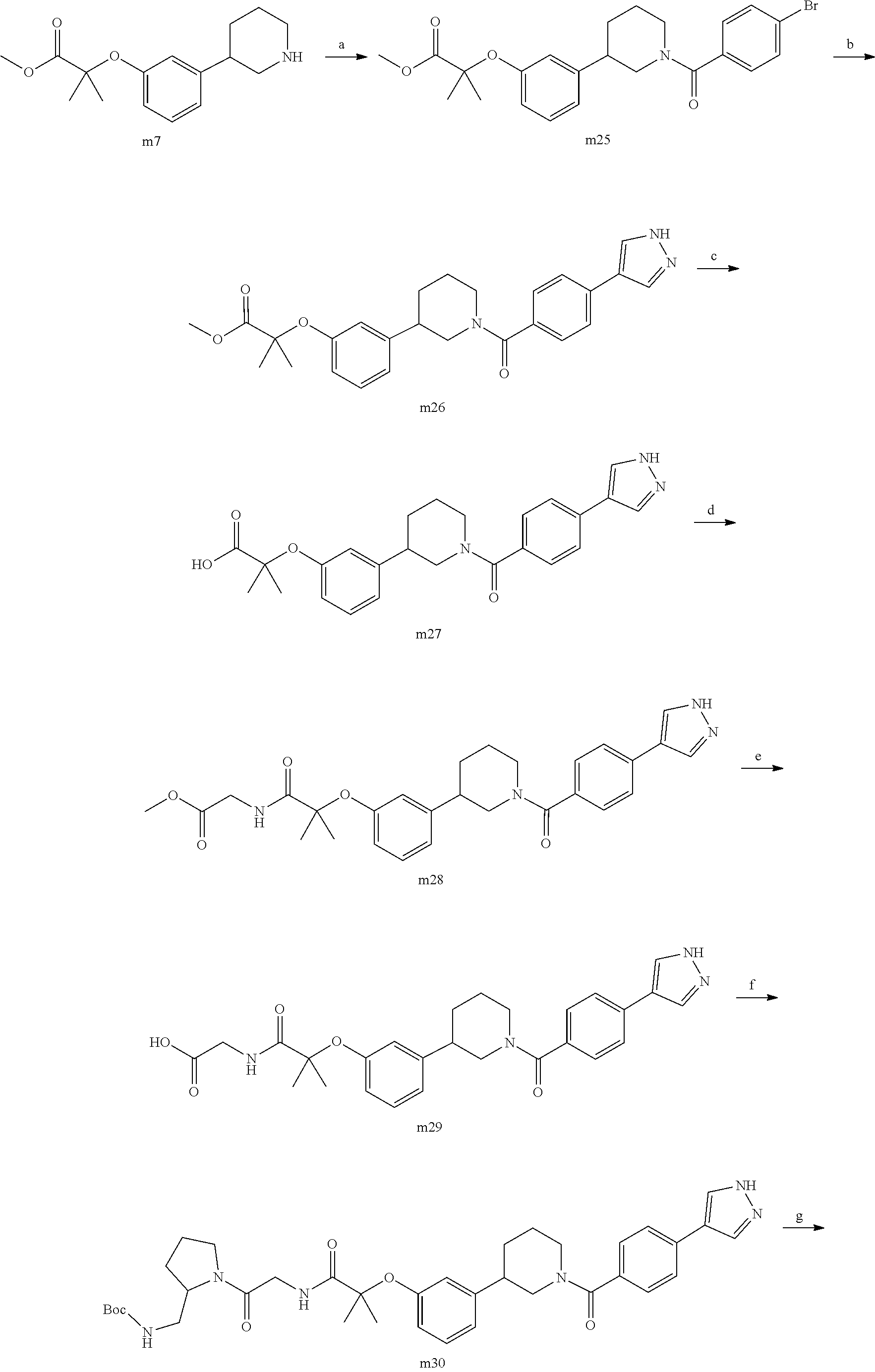

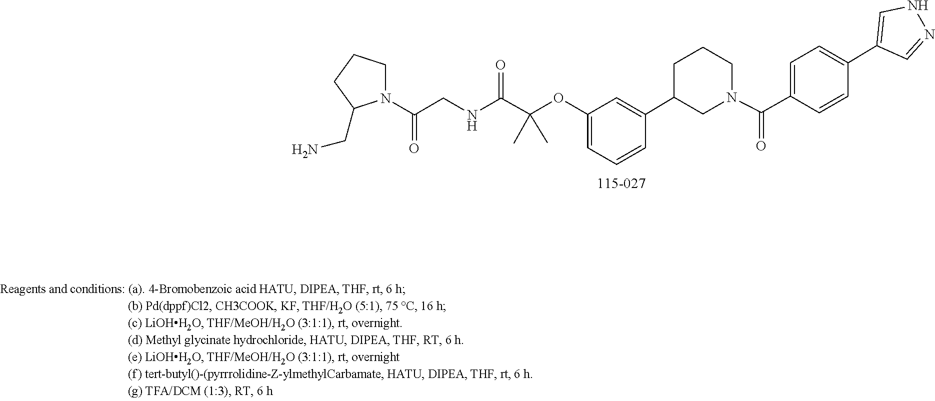 [SMILES: CC(C)(Oc1cccc(C2CCCN(C(=O)c3ccc(-c4cn[nH]c4)cc3)C2)c1)C(=O)NCC(=O)N1CCCC1CN]

### Specific Steps

Synthesis of the intermediate m25: Add the intermediate m7 (0.4 g, 1 eq) in a 100 ml dry reaction flask, dissolve in 40 ml THF, and add p-bromobenzoic acid (0.35 g, 1.2 eq). DIPEA (0.75 g, 4 eq), HATU (0.67 g, 1.2 eq Room temperature stirring reaction for 6 h. TLC plate (petroleum ether:ethyl acetate=10:1) monitored the reaction, added 30 ml of water to the reaction solution, DCM (30 ml×3) extracted and separated, collected the organic phase, washed with 40 ml saturated saline, dried with anhydrous Na₂SO₄, and the residual solution was chromatized by silica gel column[V (petroleum ether): V (ethyl acetate)=10: 1 as eluent] The yellowish viscous solid product m25 (0.47 g, yield 71%) was purified.

Synthesis of the intermediate m26: Add the intermediate m25 (0.47 g, 1 eq) in a 100 ml dry reaction flask, dissolve with 30 ml of dioxane, and then add potassium acetate (0.4 g, 4 eq). (1-Boc-pyrazole-pinnaol borate, 0.34 g, 1.1 eq), Pd(dppf)C₁₂ (0.07 g, 0.01 eq), add 5 ml of water, stir the reaction at 80 °C under the protection of N2 for 36 h, TLC plate (petroleum ether: ethyl acetate=1:1) monitor the reaction, then cool the reaction solution to room temperature, filter to remove insolubles, and concentrate the filtrate, The residue was purified by silica column chromatography [V (petroleum ether): V (ethyl acetate)=1: 1 as eluent] to obtain the intermediate m26 (0.28 g, yield 62%).

Synthesis of the intermediate m27: add compound m26 (0.28 g, 1 eq) in a 50 ml dry reaction flask, dissolve with 20 ml THF, dissolve lithium hydroxide (0.11 g, 4 eq) in 5 ml of water, and add 5 ml methanol was mixed well, and finally added to the reaction solution, and the reaction was stirred at room temperature for 4 h. TLC plate (petroleum ether: ethyl acetate=1:1) to monitor the reaction, concentrate the reaction solution, add 20 ml of water to the residual solution, adjust the PH of about 4-5 with 1 M HCl, extract with ethyl acetate (30 ml×3), merge with organic phases, and wash with 40 ml saturated saline, The yellowish solid compound m27 (0.27 g, yield 99%) was obtained by drying the anhydrous Na₂SO₄ three times to concentrate the organic phase.

Synthesis of intermediate m28: add intermediate m27 (0.27 g, 1 eq) in a 100 ml dry reaction flask, dissolve in 40 ml THF, and add methyl glycine hydrochloride (0.1 g, 1.2 eq), DIPEA (0.32 g, 4 eq), HATU (0.28 g, 1.2 eq Room temperature stirring reaction for 6 h. TLC plate (petroleum ether:ethyl acetate=1:1) monitored the reaction, 30 ml of water was added to the reaction solution, DCM (30 ml×3) was extracted and separated, the organic phase was collected, washed with 40 ml saturated saline, dried with anhydrous Na₂SO₄, and the residue was chromatized by silica gel column[V (petroleum ether): V (ethyl acetate)=1: 1 as eluent] The yellowish viscous solid product m28 (0.26 g, yield 81.2%) was purified.

Synthesis of the intermediate m29: Add compound m28 (0.26 g, leg) in a 50 ml dry reaction flask, dissolve with 20 ml THF, dissolve lithium hydroxide (0.09 g, 4 eq) in 5 ml of water, and add 5 ml methanol was mixed well, and finally added to the reaction solution, and the reaction was stirred at room temperature for 4 h. TLC plate (petroleum ether: ethyl acetate=1:1) to monitor the reaction, concentrate the reaction solution, add 20 ml of water to the residual solution, adjust the PH of about 4-5 with 1 M HCl, extract with ethyl acetate (30 ml×3), merge with organic phases, and wash with 40 ml saturated saline, The yellowish solid compound m29 (0.25 g, yield 99%) was obtained by drying the anhydrous Na₂SO₄ three times to concentrate the organic phase.

Synthesis of intermediate m30: Add intermediate m29 (0.25 g, leg) in a 100 ml dry reaction flask, dissolve in 15 ml THF, and add tert-butyl-(pyrrolidine-2-ylmethyl)Carbamate (0.13 g, 1.2 eq), DIPEA (0.32 g, 1.2 eq). HATU (0.25 g, 1.2 eq) was stirred at room temperature for 6 h. TLC plate (ethyl acetate) monitored the reaction, concentrated the reaction solution, added 20 ml of water to the residual solution, extracted with DCM (30 ml×3), merged with organic phases, washed with 40 ml saturated saline, dried with anhydrous Na₂SO₄, and the residue was chromatized by silica gel column [ethyl acetate is the eluent The yellowish solid product m30 (0.22 g, yield 65%) was purified.

Synthesis of 115-027: m30 (100 mg) was added to DCM (15 mL) solution, then 5 mL TFA was added to the reaction solution drops, stirred at room temperature for 6 h, TLC monitored until the reaction was 50 completed, evaporated and concentrated filtrate under reduced pressure, and the crude product was obtained after evaporation drying, water was added, and DCM was extracted. The organic layer was washed with saturated NaCl aqueous solution, then dried with anhydrous Na₂SO₄ solids, and the reaction solution was evaporated and concentrated under reduced pressure to obtain a crude product. The crude product was purified by reversed-phase preparation (water/acetonitrile/trifluoroacetic acid) to obtain product 115-027 preparation solution, and frozen in the refrigerator at −80 °C for 6 h Then the freeze-dried white solid end product 115-027, 28 mg, yield 35.88%; HPLC >95%.

### (2) Compounds 115-028, 115-029, 115-030, 115-031 were Prepared by Synthesis Method Similar to compound 115-027

### Preparation Example 5: Synthesis of Compounds 115-047, 115-028, 115-029, 115-030, 115-031

### (1) Synthesis of Compound 115-047

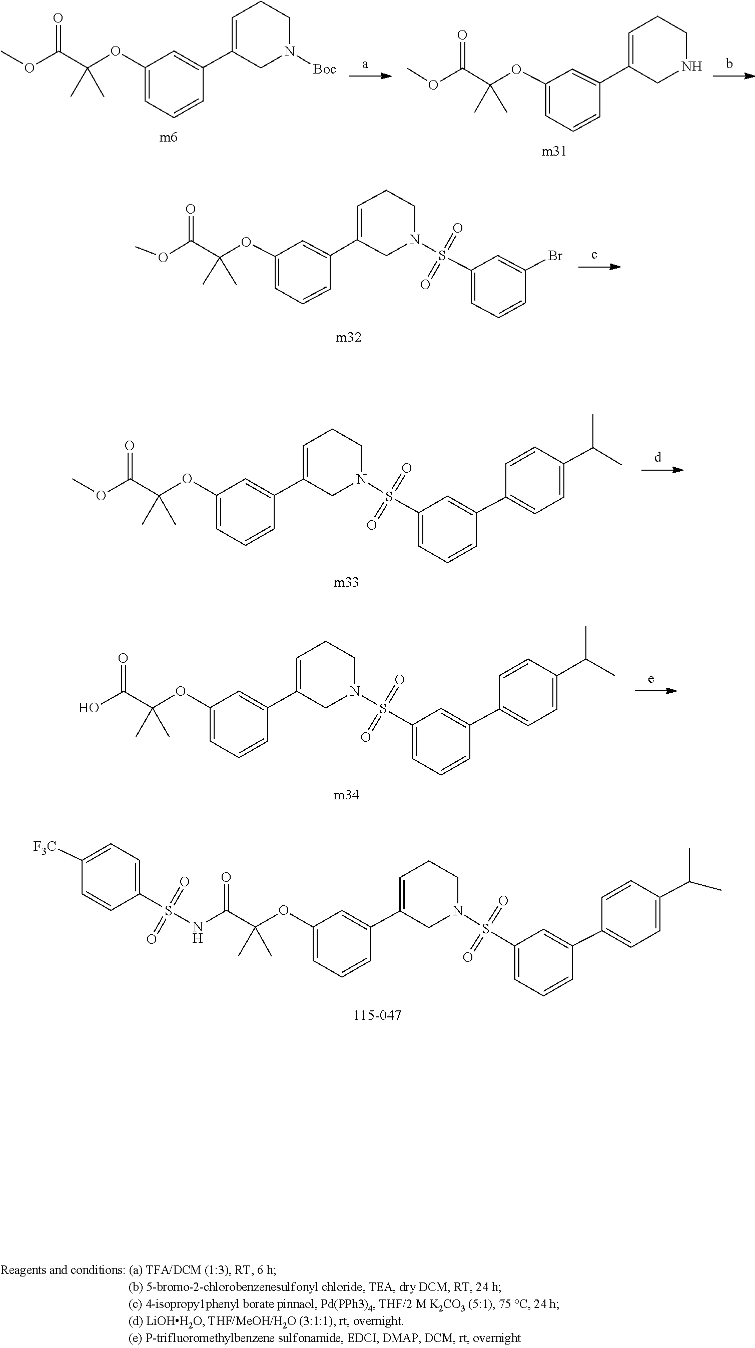 [SMILES: COC(=O)C(C)(C)Oc1cccc(C2=CCCN(C(=O)OC(C)(C)C)C2)c1 ; COC(=O)C(C)(C)Oc1cccc(C2=CCCNC2)c1 ; COC(=O)C(C)(C)Oc1cccc(C2=CCCN(S(=O)(=O)c3cccc(Br)c3)C2)c1 ; COC(=O)C(C)(C)Oc1cccc(C2=CCCN(S(=O)(=O)c3cccc(-c4ccc(C(C)C)cc4)c3)C2)c1 ; CC(C)c1ccc(-c2cccc(S(=O)(=O)N3CCC=C(c4cccc(OC(C)(C)C(=O)O)c4)C3)c2)cc1 ; CC(C)c1ccc(-c2cccc(S(=O)(=O)N3CCC=C(c4cccc(OC(C)(C)C(=O)NS(=O)(=O)c5ccc(C(F)(F)F)cc5)c4)C3)c2)cc1]

### Specific Steps

Synthesis of intermediate m31: m6 (0.3 g, leg) was added to DCM (15 mL) solution, then 5 mL TFA was added to the reaction solution, stirred at room temperature for 6 h, TLC monitored until the reaction was completed, evaporated and concentrated filtrate under reduced pressure, and the crude product m7 (crude product) was 0.29 g of light yellow oil after evaporation, with a yield of 100%.

Synthesis of intermediate m32: Intermediate products m31 (0.29 g, leg), 5-bromo-2-chlorobenzenesulfonyl chloride (0.33 g, 1.1 eq), TEA (0.31 g, 3 eq), anhydrous DCM solution (15 ml) was added to a 50 ml round-bottom flask and stirred at room temperature under nitrogen protection. TLC monitoring until the completion of the reaction, evaporation of the concentrated filtrate under reduced pressure, dilution with water, and extraction of ethyl acetate. The organic layer was dried with anhydrous Na₂SO₄ solids, and the crude product was obtained by evaporating and concentrating the filtrate under reduced pressure. The crude product was purified by silica column chromatography (petroleum ether/ethyl acetate=5:1), and the intermediate product m32 was obtained as a colorless oil of 0.35 g, with a yield of 67.31%.

Synthesis of the intermediate m33: m32 (0.30 g, 1 eq), 4-isopropylphenylenebrate pinnaol (0.35 g, 1.1 eq) and Pd(PPh3)₄ (436 09 mg, 0.19 mmol) Add to THF/2M K₂CO₃ (5:1) (15:3 mL) solution (75 °C) and stir the reaction under nitrogen protection for 12 h. TLC monitoring until the reaction is complete, decompression evaporation of the concentrated filtrate, and the resulting residue is added with water and ethyl acetate for extraction. The organic layer was washed with saturated NaCl solution, then dried with anhydrous Na₂SO₄ solids, and the filtrate was evaporated and concentrated under reduced pressure to obtain a crude product.

The crude product was purified by silica gel column chromatography (petroleum ether/ethyl acetate=10:1), and the intermediate product m33 was obtained as a colorless oil with a yield of 64.89% at 0.24 g.

Synthesis of intermediate m34: m33 (0.24 g, 1 eq), LiOH H₂O (69 mg, 4 eq) was added to a solution of THF/MeOH/H₂O (3:1:1) (12:4:4 mL) and stirred overnight at room temperature. TLC is monitored until the reaction is complete, and the mixture is depressurized and concentrated. The resulting residue was dissolved in ethyl acetate, adjusted to pH=2 with 1M HCl, and then extracted with ethyl acetate. The organic layer was dried with anhydrous Na₂SO₄ solid, and the reaction liquid was evaporated and concentrated under reduced pressure, and the crude product m34 was white solid, 0.23 g, which was directly used for the next reaction.

Synthesis of 115-047: m33 (0.23 g, 1 eq), p-trifluoromethylbenzene sulfonamide (89.2 mg, 1.1 eq), 40 EDCI (147 mg, 1.3 eq) DMAP (127 mg, 2 eq) was added to the DCM solution (15 ml), and the mixture was stirred overnight at room temperature for the reaction. After TIC monitoring until the reaction is completed, the mixture is adjusted to PH=2 with 1M HCl and extracted by DCM. The organic layer was washed with saturated NaCl aqueous solution, then dried with anhydrous Na₂SO₄ solids, and the reaction solution was evaporated and concentrated under reduced pressure to obtain a crude product. The crude product was purified by reversed-phase preparation (water/acetonitrile/trifluoroacetic acid) to obtain the product 115-047 preparation solution, and was frozen in the refrigerator at −80 °C for 6 h Then the white solid end product of freeze-dried by freeze-dryer was 115-047, 126 mg, with a yield of 39.34%. HPLC >95%.

### Preparation Example 6: Synthesis of Compounds 115-024, 115-025, 115-026

### (1) Synthesis of Compound 115-025

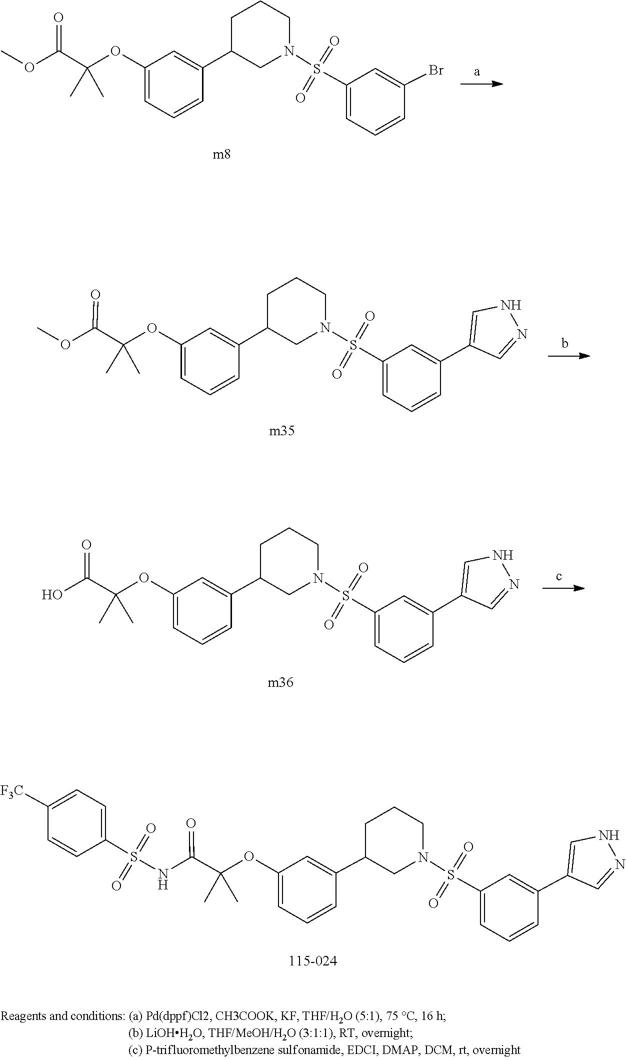 [SMILES: COC(=O)C(C)(C)Oc1cccc(C2CCCN(S(=O)(=O)c3cccc(Br)c3)C2)c1 ; COC(=O)C(C)(C)Oc1cccc(C2CCCN(S(=O)(=O)c3cccc(-c4cn[nH]c4)c3)C2)c1 ; CC(C)(Oc1cccc(C2CCCN(S(=O)(=O)c3cccc(-c4cn[nH]c4)c3)C2)c1)C(=O)O ; CC(C)(Oc1cccc(C2CCCN(S(=O)(=O)c3cccc(-c4cn[nH]c4)c3)C2)c1)C(=O)NS(=O)(=O)c1ccc(C(F)(F)F)cc1]

### Specific Steps

Synthesis of intermediate m35: raw materials m8 (0.1 g, 1 eq), M2 (0.0652 g, 1.1 eq), potassium acetate (0.0791 g, 4 eq), potassium fluoride (0.0468 g, 4 eq), and catalyst Pd (dppf) Cl2 0. 0.0148 g, 0.1 eq) was added to THF/H₂O (24 mL) and heated to 75 °C for 16 h under the protection of nitrogen. TLC is monitored until the reaction is completed, and the concentrated filtrate is evaporated under reduced pressure, and the crude product is obtained after evaporation. The crude product was purified by silica column chromatography (petroleum ether/ethyl acetate=6:1), and the intermediate product m35 was a light yellow oil, 0.0883 g, with a yield of 90.7%.

Synthesis of intermediate m36: m35 (0.0883 g, leg), LiOH H₂O (0.0176 g, 4 eq) was added to the solution of THF/MeOH/H₂O (3:1:1) (12:4:4 mL) and stirred overnight at room temperature. TLC is monitored until the reaction is complete, and the mixture is depressurized and concentrated. The resulting residue was dissolved in ethyl acetate, adjusted to pH=2 with 1MHCl, and then extracted with ethyl acetate.

The organic layer was dried with anhydrous Na₂SO₄ solid, and the crude product m36 was evaporated and concentrated under reduced pressure, and the crude product m36 was obtained as a white solid, 0.0788 g, which was directly used for the next reaction.

Synthesis of 115-025: m36 (0.0788 g, leg), p-trifluoromethylbenzene sulfonamide (0.0407 mg, 1.1 eq), EDCI (0.0331 g,). 1.3 eq), DMAP (0.0401 g, 2 eq) was added to the DCM solution (15 ml), and the mixture was stirred overnight at room temperature for the reaction. After TIC monitoring until the reaction is completed, the mixture is adjusted to PH=2 with 1M HCl and extracted by DCM. The organic layer was washed with saturated NaCl aqueous solution, then dried with anhydrous Na₂SO₄ solids, and the reaction solution was evaporated and concentrated under reduced pressure to obtain a crude product. The crude product was purified by reversed-phase preparation (water/acetonitrile/trifluoroacetic acid) to obtain product 115-025 preparation solution, and was refrigerated at −80 °C for 6 h Then the freeze-dried white solid end product 115-025, 0.0262 g, yield 23.57%; HPLC >95%.

### (2) Compounds 115-024 and 115-026 were Prepared by Synthesis Method Similar to Compound 115-025

### Preparation Example 7: Synthesis of Compound 115-049

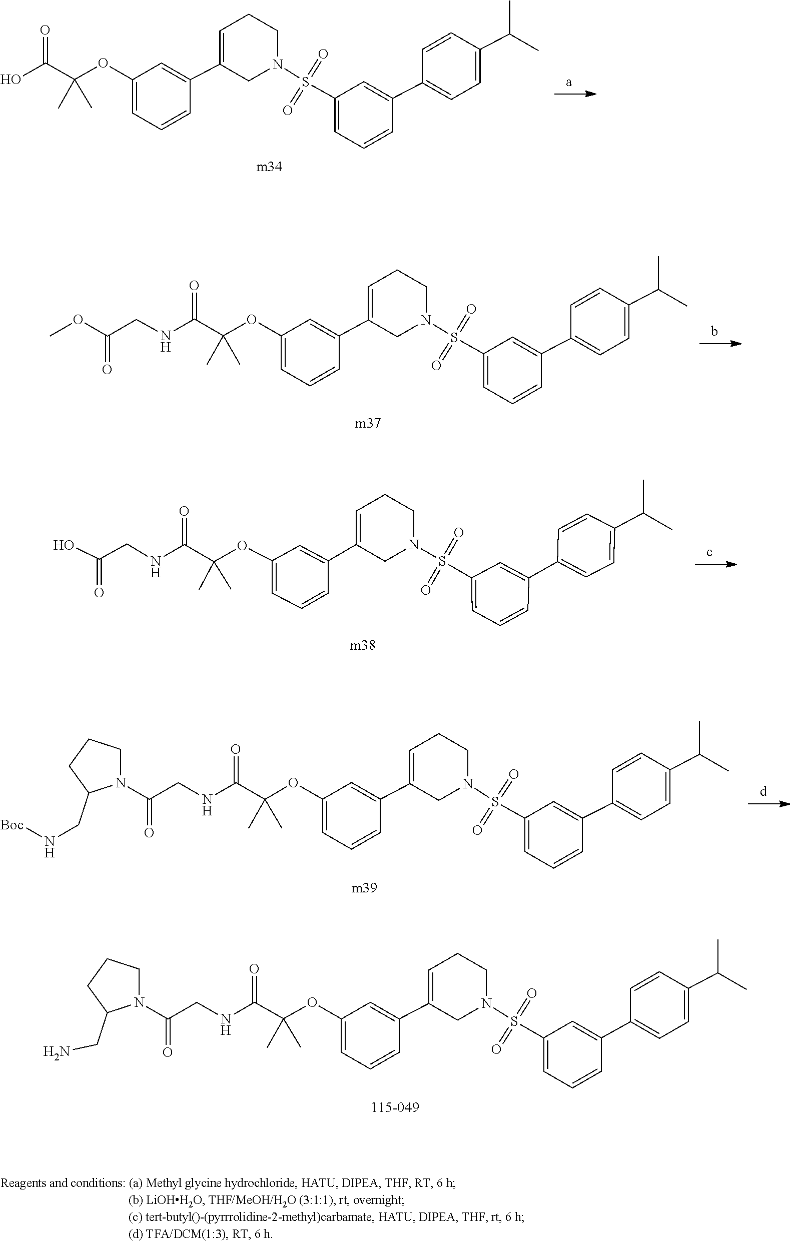

### Specific Steps

Synthesis of intermediate m37: Add intermediate m34 (0.28 g, leg) to 100 ml dry reaction flask, dissolve with 40 ml THF, and add methyl glycine hydrochloride (0.09 g, 1.2 eq). DIPEA (0.62 g, 8 eq) and HATU (0.23 g, leg) were stirred at room temperature for 6 h. TLC plate (petroleum ether: ethyl acetate=1:1) monitored the reaction, 30 ml of water was added to the reaction solution, DCM (30 ml×3) was extracted and separated, the organic phase was collected, washed with 40 ml saturated saline, dried with anhydrous Na₂SO₄, and the residual solution was chromatized by silica gel column[V (petroleum ether): V (ethyl acetate)=1: 1 as eluent] The yellowish viscous solid product m37 (0.33 g, yield 98.3%) was purified.

Synthesis of the intermediate m38: add the compound m37 (0.33 g, leg) in a 50 ml dry reaction flask, dissolve it in 20 ml THF, dissolve lithium hydroxide (0.124 g, 5 eq) in 5 ml of water, then add 5 ml methanol and mix well, Finally, it was added to the reaction solution and stirred at room temperature for 4 h. TLC plate (petroleum ether: ethyl acetate=1:1) to monitor the reaction, concentrate the reaction solution, add 20 ml of water to the residual solution, adjust the PH about 4-5 with 1 M HCl, extract it with ethyl acetate (30 ml×3), merge with organic phases, and wash with 40 ml saturated saline, The yellowish solid compound m38 (0.29 g, yield 93.54%) was obtained by drying anhydrous Na₂SO₄ three times to concentrate the organic phase.

Synthesis of intermediate m39: Add intermediate m38 (0.29 g, 1 eq) in a 100 ml dry reaction flask, dissolve with 25 ml THF, and add tert-butyl-(pyrrolidine-2-ylmethyl group). Carbamate (0.13 g, 1.2 eq), DIPEA (0.572 g, 8 eq), HATU (0.21 g, leg) at room temperature for 6 h. TLC plate (ethyl acetate) monitored the reaction, concentrated the reaction solution, added 20 ml of water to the residual solution, extracted with DCM (30 ml×3), merged with organic phase, washed with 40 ml saturated saline, dried with anhydrous Na₂SO₄, and the residual solution was chromatized by silica gel column [ethyl acetate is the eluent The yellowish solid product m39 (0.23 g, yield 53%) was purified.

Synthesis of 115-049: m39 (100 mg) was added to DCM (15 mL) solution, then 5 mL TFA was added to the reaction solution drops, stirred at room temperature for 6 h, TLC monitored until the reaction was completed, evaporated and concentrated filtrate under reduced pressure, and the crude product was obtained after evaporation, water was added, and DCM was extracted. The organic layer was washed with saturated NaCl aqueous solution, then dried with anhydrous Na₂SO₄ solids, and the reaction solution was evaporated and concentrated under reduced pressure to obtain a crude product. The crude product was purified by reversed-phase preparation and purification (water/acetonitrile/trifluoroacetic acid) to obtain product 115-049, and the white solid final product was frozen at −80′ C for 6 h and then freeze-dried by freeze dryer 115-049, 43 mg, with a yield of 49.60%; HPLC >95%.

### Preparation Example 8: Synthesis of Compounds 115-052 and 115-055

### (1) Synthesis of Compound 115-052

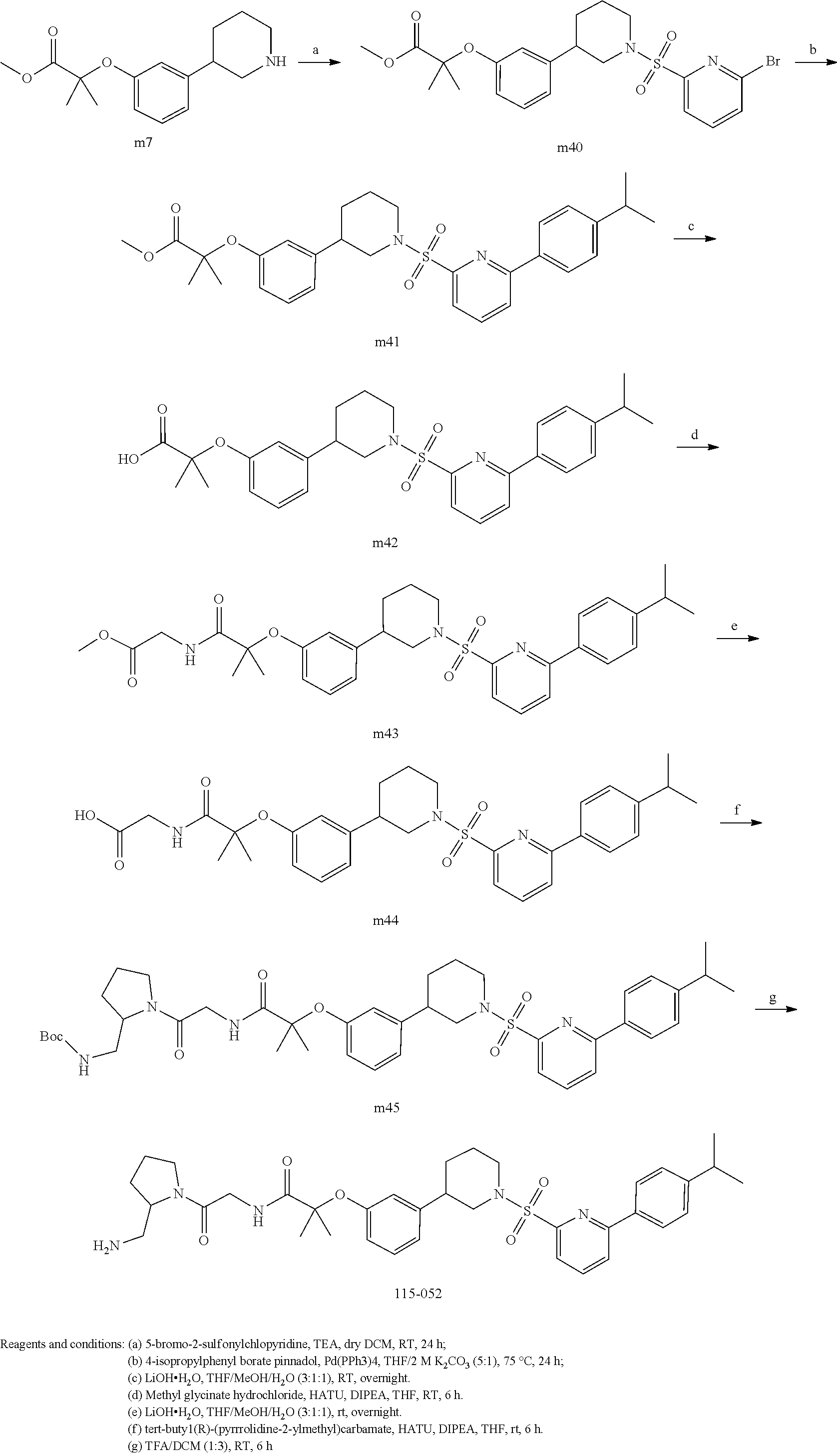

### Specific Steps

Synthesis of intermediate m40: Intermediate products m7 (0.6 g, leg), 5-bromo-2-sulfonylchloropyridine (0.7 g, 1.1 eq), TEA (0.62 g, 3 eq), anhydrous DCM solution (15 ml).) into a 50 ml round-bottom flask and stir the reaction at room temperature under nitrogen protection. TLC monitoring until the completion of the reaction, evaporation of the concentrated filtrate under reduced pressure, dilution with water, and extraction of ethyl acetate. The organic layer was dried with anhydrous Na₂SO₄ solids, and the crude product was obtained by evaporating and concentrating the filtrate under reduced pressure. The crude product was purified by silica column chromatography (petroleum ether/ethyl acetate=5:1), and the intermediate product m8 was a colorless oil with a yield of 0.77 g and a yield of 70.64%.

Synthesis of the intermediate m41: m40 (0.30 g, 1 eq), 4-isopropylphenyl borate pinnaol ester (0.15 g, 1.1 eq), and Pd(PPh3)₄ (216.09 mg, 0.19 mmol) were added to THF/2M K₂CO₃ (5:1) (15:). 3 mL) In solution (75 °C), stir the reaction under nitrogen protection for 12 h. TLC monitoring until the reaction is complete, decompression evaporation of the concentrated filtrate, and the resulting residue is added with water and ethyl acetate for extraction. The organic layer was washed with saturated NaCl solution, then dried with anhydrous Na₂SO₄ solids, and the filtrate was evaporated and concentrated under reduced pressure to obtain a crude product. The crude product was purified by silica gel column chromatography (petroleum ether/ethyl acetate=10:1), and the intermediate product m9 was obtained as a colorless oil with a yield of 74.19% of 0.23 g.

Synthesis of intermediate m42: m41 (0.23 g, 1 eq), LiOH H₂O (69 mg, 4 eq) was added to a solution of THF/MeOH/H₂O (3:1:1) (12:4:4 mL) and stirred overnight at room temperature. TLC is monitored until the reaction is complete, and the mixture is depressurized and concentrated. The resulting residue was dissolved in ethyl acetate, adjusted to pH=2 with 1MHCl, and then extracted with ethyl acetate. The organic layer was dried with anhydrous Na₂SO₄ solid, and the reaction liquid was evaporated and concentrated under reduced pressure, and the crude product m10 was white solid, 0.26 g, which was directly used for the next reaction.

Synthesis of intermediate m43: add intermediate m42 (0.26 g, 0.57 mmol) in a 100 ml dry reaction flask, dissolve with 40 ml THF, and add methyl glycinate hydrochloride (0.12 g, 0.93 mmol), DIPEA (0.40 g, 3.09 mmol), HATU (0.32 g, 0.85 mmol Room temperature stirring reaction for 6 h. TLC plate (petroleum ether: ethyl acetate=1:1) monitored the reaction, 30 ml of water was added to the reaction solution, DCM (30 ml×3) was extracted and separated, the organic phase was collected, washed with 40 ml saturated saline, dried with anhydrous Na₂SO₄, and the residual solution was chromatized by silica gel column[V (petroleum ether): V (ethyl acetate)=1: 1 as eluent] purified yellowish viscous solid product m11 (0.37 g, yield 81%).

Synthesis of the intermediate m44: Add compound m43 (0.37 g, 0.63 mmol) in a 50 ml dry reaction flask, dissolve with 20 ml THF, dissolve lithium hydroxide (0.12 g, 2.52 mmol) in 5 ml water, and add 5 ml methanol was mixed well, and finally added to the reaction solution, and the reaction was stirred at room temperature for 4 h. TLC plate (petroleum ether: ethyl acetate=1:1) to monitor the reaction, concentrate the reaction solution, add 20 ml of water to the residual solution, adjust the PH about 4-5 with 1 M HCl, extract it with ethyl acetate (30 ml×3), merge with organic phases, and wash with 40 ml saturated saline, The yellowish solid compound m12 (0.33 g, yield 99%) was obtained by drying the anhydrous Na₂SO₄ three times to concentrate the organic phase.

Synthesis of the intermediate m45: Add the intermediate m44 (0.32 g, 0.57 mmol) in a 100 ml dry reaction flask, dissolve in 15 ml THF, and add tert-butyl(R)-(pyrrolidine-2-ylmethyl)Carbamate (0.14 g, 0.69 mmol), DIPEA (0.30 g, 2.30 mmol), HATU (0.24 g, 0.63 mmol) at room temperature stirring for 6 h.

TLC plate (ethyl acetate) monitored the reaction, concentrated the reaction solution, added 20 ml of water to the residual solution, extracted with DCM (30 ml×3), merged with organic phase, washed with 40 ml saturated saline, dried with anhydrous Na₂SO₄, and the residual solution was chromatized by silica gel column [ethyl acetate is the eluent The yellowish solid product m13 (0.32 g, yield 74%) was purified.

Synthesis of 115-052: m45 (100 mg) was added to DCM (15 mL) solution, then 5 mL TFA was added to the reaction solution, stirred at room temperature for 6 h, TLC monitored until the reaction was completed, evaporated and concentrated filtrate under reduced pressure, and the crude product was obtained after evaporation, water was added, and DCM was extracted. The organic layer was washed with saturated NaCl aqueous solution, then dried with anhydrous Na₂SO₄ solids, and the reaction solution was evaporated and concentrated under reduced pressure to obtain a crude product. The crude product was purified by 50 reversed-phase preparation (water/acetonitrile/trifluoroacetic acid) to obtain the product 115-052 preparation solution, and the white solid final product was frozen at −80′ C for 6 h and then freeze-dried by freeze dryer 115-052, 44 mg, with a yield of 52.11%; HPLC >95%.

### (2) Compound 115-055 was Prepared by a Synthesis Method Similar to Compound 115-052

### Preparation Example 9: Characterization of Compounds

The characterization results of the compounds prepared above are shown in the table below:

| | |
|---|---|
| 115-010 | 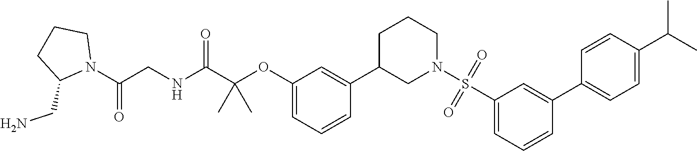 [SMILES: CC(C)c1ccc(-c2cccc(S(=O)(=O)N3CCCC(c4cccc(OC(C)(C)C(=O)NCC(=O)N5CCC[C@H]5CN)c4)C3)c2)cc1] |
| | N-(2-((S)-2-(aminomethyl)pyrrrolidine-1-yl)-2-oxoethyl)-2-(3-(1-(((4′-isopropyl-[1,1′-biphenyl] ]-3-yl) |
| | sulfonylpiperidine-3-yl)phenoxy)-2-methylpropionamide |
| | ^{1H NMR (400 MHz, DMSO-}d6) δ 8.28 (t, J = 5.3 Hz, 1H), 7.93 (dd, J = 6.9, 1.5 Hz, 2H), 7.83-7.75 (m, 1H), 7.63-7.56 |
| | (m, 2H), 7.60-7.48 (m, 1H), 7.33 (d, J = 8.1 Hz, 2H), 7.25-7.17 (m, 1H), 6.96-6.88 (m, 1H), 6.79 (t, J = 2.2 |
| | Hz, 1H), 6.74 (dt, J = 7.0, 1.6 Hz, 1H), 4.48 (t, J = 6.8 Hz, 2H), 4.22 (dd, J = 5.1, 1.0 Hz, 2H), 3.95-3.86 (m, |
| | 1H), 3.78-3.68 (m, 1H), 3.51 (ddd, J = 12.2, 4.9, 3.1 Hz, 1H), 3.40-3.31 (m, 2H), 3.20 (dd, J = 12.5, 5.1 Hz, |
| | 1H), 2.91 (ddd, J = 12.6, 9.1, 5.9 Hz, 2H), 2.65 (dtd, J = 11.3, 6.8, 4.6 Hz, 1H), 2.51 (p, J = 5.4 Hz, 1H), 2.46- |
| | 2.35 (m, 1H), 2.12-2.00 (m, 1H), 1.97-1.83 (m, 2H), 1.77 (dddd, J = 11.2, 9.6, 7.4, 4.4 Hz, 2H), 1.67-1.53 |
| | (m, 8H), 1.39 (dddd, J = 12.2, 8.4, 6.4, 2.7 Hz, 1H), 1.22 (d, J = 6.5 Hz, 3H), 1.17 (d, J = 6.5 Hz, 3H). ESI-MS |
| | calculated value C37H48N4O5S[M + H] ⁺: 661.88, measured value: 661.43 |
| 115-011 | 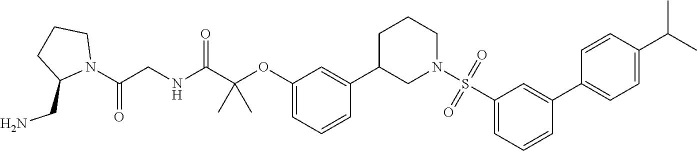 [SMILES: CC(C)c1ccc(-c2cccc(S(=O)(=O)N3CCCC(c4cccc(OC(C)(C)C(=O)NCC(=O)N5CCC[C@@H]5CN)c4)C3)c2)cc1] |
| | N-(2-((R)-2-(aminomethyl)pyrrolidin-1-yl)-2-oxoethyl)-2-(3-(1-((4′-isopropyl-[1,1′-biphenyl.)-2-(4′-isopropyl- |
| | [1,1′-biphenyl.″ ]-3-yl)sulfonylpiperidine-3-yl)phenoxy)-2-methylpropionamide |
| | ^{1H NMR (400 MHz, DMSO-}d6) δ 8.28 (t, J = 5.3 Hz, 1H), 7.93 (dd, J = 6.9, 1.5 Hz, 2H), 7.83-7.75 (m, 1H), 7.63-7.56 |
| | (m, 2H), 7.60-7.48 (m, 1H), 7.33 (d, J = 8.1 Hz, 2H), 7.25-7.17 (m, 1H), 6.96-6.88 (m, 1H), 6.79 (t, J = 2.2 |
| | Hz, 1H), 6.74 (dt, J = 7.0, 1.6 Hz, 1H), 4.48 (t, J = 6.8 Hz, 2H), 4.22 (dd, J = 5.1, 1.0 Hz, 2H), 3.95-3.86 (m, |
| | 1H), 3.78-3.68 (m, 1H), 3.51 (ddd, J = 12.2, 4.9, 3.1 Hz, 1H), 3.40-3.31 (m, 2H), 3.20 (dd, J = 12.5, 5.1 Hz, |
| | 1H), 2.91 (ddd, J = 12.6, 9.1, 5.9 Hz, 2H), 2.65 (dtd, J = 11.3, 6.8, 4.6 Hz, 1H), 2.51 (p, J = 5.4 Hz, 1H), 2.46- |
| | 2.35 (m, 1H), 2.12-2.00 (m, 1H), 1.97-1.83 (m, 2H), 1.77 (dddd, J = 11.2, 9.6, 7.4, 4.4 Hz, 2H), 1.67-1.53 |
| | (m, 8H), 1.39 (dddd, J = 12.2, 8.4, 6.4, 2.7 Hz, 1H), 1.22 (d, J = 6.5 Hz, 3H), 1.17 (d, J = 6.5 Hz, 3H) .. ESI-MS |
| | calculated value C37H48N4O5S[M + H] ⁺: 661.88, measured value: 661.47 |
| 115-012 | 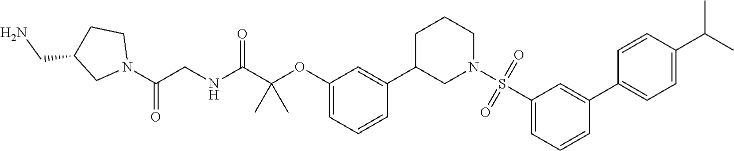 [SMILES: CC(C)c1ccc(-c2cccc(S(=O)(=O)N3CCCC(c4cccc(OC(C)(C)C(=O)NCC(=O)N5CC[C@@H](CN)C5)c4)C3)c2)cc1] |
| | N-(2-((S)-3-(aminomethyl)pyrrrolidine-1-yl)-2-oxyethyl)-2-(3-(1-((4′-isopropyl-[1,1′-biphenyls.)-(4′-isopropyl- |
| | [1,1′-biphenyls.)″ ]-3-yl)sulfonylpiperidine-3-yl)phenoxy)-2-methylpropionamide |
| | ^{1H NMR (400 MHz, DMSO-}d6) δ 8.28 (t, J = 5.0 Hz, 1H), 7.93 (dd, J = 7.0, 1.5 Hz, 2H), 7.83-7.75 (m, 1H), 7.63-7.56 |
| | (m, 2H), 7.56-7.47 (m, 1H), 7.33 (d, J = 8.1 Hz, 2H), 7.25-7.17 (m, 1H), 6.96-6.88 (m, 1H), 6.79 (t, J = 2.2 |
| | Hz, 1H), 6.74 (dt, J = 7.0, 1.6 Hz, 1H), 3.84 (d, J = 5.0 Hz, 2H), 3.73 (ddd, J = 12.3, 7.1, 5.3 Hz, 1H), 3.56- |
| | 3.46 (m, 2H), 3.40-3.30 (m, 2H), 3.30-3.16 (m, 2H), 2.91 (ddd, J = 12.6, 9.1, 5.9 Hz, 2H), 2.73-2.61 (m, |
| | 1H), 2.61-2.50 (m, 1H), 2.27 (ddt, J = 12.5, 7.3, 5.1 Hz, 1H), 2.01 (dddd, J = 12.5, 7.5, 5.2, 3.1 Hz, 1H), 1.98- |
| | 1.84 (m, 2H), 1.71 (t, J = 6.3 Hz, 2H), 1.67-1.53 (m, 8H), 1.47-1.33 (m, 1H), 1.22 (d, J = 6.5 Hz, 3H), 1.17 |
| | (d, J = 6.5 Hz, 3H). ESI-MS calculated value C37H48N4O5S[M + H]⁺: 661.88, measured value: 661.49 |
| 115-013 | 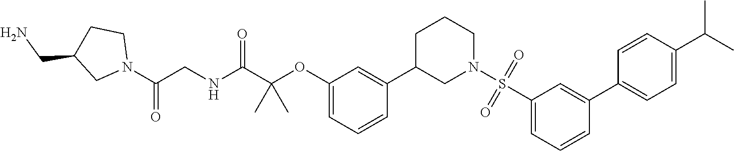 [SMILES: CC(C)c1ccc(-c2cccc(S(=O)(=O)N3CCCC(c4cccc(OC(C)(C)C(=O)NCC(=O)N5CC[C@H](CN)C5)c4)C3)c2)cc1] |
| | N-(2-((S)-3-(aminomethyl)pyrrrolidine-1-yl)-2-oxyethyl)-2-(3-(1-((4′-isopropyl-[1,1′-biphenyls.)-(4′-isopropyl- |
| | [1,1′-biphenyls.)″ ]-3-yl)sulfonylpiperidine-3-yl)phenoxy)-2-methylpropionamide |
| | ^{1H NMR (400 MHz, DMSO-}d6) δ 8.28 (t, J = 5.0 Hz, 1H), 7.93 (dd, J = 7.0, 1.5 Hz, 2H), 7.83-7.75 (m, 1H), 7.63-7.56 |
| | (m, 2H), 7.56-7.47 (m, 1H), 7.33 (d, J = 8.1 Hz, 2H), 7.25-7.17 (m, 1H), 6.96-6.88 (m, 1H), 6.79 (t, J = 2.2 |
| | Hz, 1H), 6.74 (dt, J = 7.0, 1.6 Hz, 1H), 3.84 (d, J = 5.0 Hz, 2H), 3.73 (ddd, J = 12.3, 7.1, 5.3 Hz, 1H), 3.56- |
| | 3.46 (m, 2H), 3.40-3.30 (m, 2H), 3.30-3.16 (m, 2H), 2.91 (ddd, J = 12.6, 9.1, 5.9 Hz, 2H), 2.73-2.61 (m, |
| | 1H), 2.61-2.50 (m, 1H), 2.27 (ddt, J = 12.5, 7.3, 5.1 Hz, 1H), 2.01 (dddd, J = 12.5, 7.5, 5.2, 3.1 Hz, 1H), 1.98- |
| | 1.84 (m, 2H), 1.71 (t, J = 6.3 Hz, 2H), 1.67-1.53 (m, 8H), 1.47-1.33 (m, 1H), 1.22 (d, J = 6.5 Hz, 3H), 1.17 |
| | (d, J = 6.5 Hz, 3H). ESI-MS calculated value C37H48N4O5S[M + H] ⁺: 661.88, measured value: 661.44 |
| 115-014 | 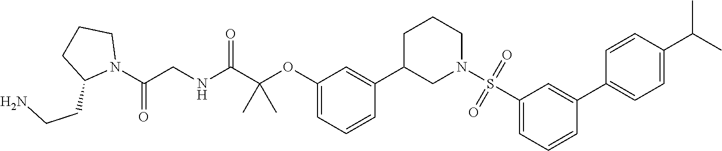 [SMILES: CC(C)c1ccc(-c2cccc(S(=O)(=O)N3CCCC(c4cccc(OC(C)(C)C(=O)NCC(=O)N5CCC[C@H]5CCN)c4)C3)c2)cc1] |
| | N-(2-((S)-2-(2-aminoethyl)pyrrolidin-1-yl)-2-oxyethyl)-2-(3-(1-((4′-isopropyl-[1,1′-Biphenyl]-3-yl) |
| | sulfonylpiperidine-3-yl)phenoxy)-2-methylpropionamide). |
| | ^{1H NMR (400 MHz, DMSO-}d6) δ 8.28 (t, J = 5.3 Hz, 1H), 7.93 (dd, J = 6.9, 1.5 Hz, 2H), 7.83-7.75 (m, 1H), 7.63-7.56 |
| | (m, 2H), 7.60-7.48 (m, 1H), 7.33 (d, J = 8.1 Hz, 2H), 7.25-7.17 (m, 1H), 6.96-6.88 (m, 1H), 6.79 (t, J = 2.2 |
| | Hz, 1H), 6.74 (dt, J = 7.0, 1.6 Hz, 1H), 4.95 (qd, J = 6.5, 3.9 Hz, 1H), 4.46 (t, J = 6.7 Hz, 2H), 4.22 (dd, J = |
| | 5.1, 1.0 Hz, 2H), 3.78-3.68 (m, 1H), 3.51 (ddd, J = 12.1, 4.9, 3.1 Hz, 1H), 3.40-3.31 (m, 2H), 3.20 (dd, J = |
| | 12.5, 5.1 Hz, 1H), 3.10-2.85 (m, 5H), 2.56-2.46 (m, 1H), 2.34-2.22 (m, 1H), 2.12-1.98 (m, 2H), 1.97- |
| | 1.83 (m, 2H), 1.76 (dddd, J = 11.4, 9.7, 8.4, 4.6 Hz, 2H), 1.67-1.53 (m, 8H), 1.39 (dddd, J = 12.2, 8.4, 6.4, |
| | 2.7 Hz, 1H), 1.22 (d, J = 6.5 Hz, 3H), 1.17 (d, J = 6.5 Hz, 3H). ESI-MS: Calculated value C37H48N4O5S [M + H]⁺: |
| | 661.88, Measured value: 661.44 |
| 115-016 | 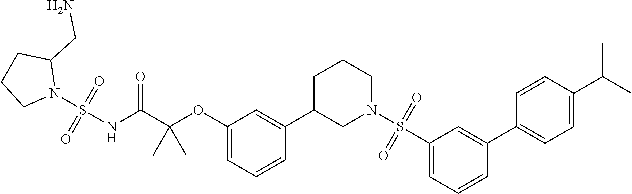 [SMILES: CC(C)c1ccc(-c2cccc(S(=O)(=O)N3CCCC(c4cccc(OC(C)(C)C(=O)NS(=O)(=O)N5CCCC5CN)c4)C3)c2)cc1] |
| | N-((2-(aminomethyl)pyrrrolidine-1-yl)sulfonyl)-2-(3-(1-(((4′-isopropyl-[1,1′-biphenyls.)-2-(4′-isopropyl-[1,1′- |
| | biphenyls.)″]-3-yl)sulfonylpiperidine-3-yl)phenoxy)-2-methylpropionamide |
| | ^{1H NMR (400 MHz, DMSO-}d6) δ 7.93 (dd, J = 6.9, 1.5 Hz, 2H), 7.83-7.75 (m, 1H), 7.63-7.56 (m, 2H), 7.60-7.47 |
| | (m, 1H), 7.33 (d, J = 8.1 Hz, 2H), 7.25-7.17 (m, 1H), 7.00 (s, 1H), 6.96-6.88 (m, 1H), 6.79 (t, J = 2.2 Hz, |
| | 1H), 6.74 (dt, J = 7.0, 1.6 Hz, 1H), 4.48 (t, J = 6.6 Hz, 2H), 4.19-4.09 (m, 1H), 4.01-3.91 (m, 1H), 3.40- |
| | 3.31 (m, 2H), 3.20 (dd, J = 12.5, 5.1 Hz, 1H), 2.91 (ddd, J = 12.6, 9.1, 5.9 Hz, 2H), 2.75-2.65 (m, 1H), 2.51 |
| | (tdd, J = 6.8, 5.1, 3.2 Hz, 2H), 2.27 (dtd, J = 10.8, 6.5, 4.4 Hz, 1H), 2.09-1.96 (m, 1H), 1.96-1.84 (m, 1H), |
| | 1.80-1.66 (m, 2H), 1.70-1.53 (m, 9H), 1.47-1.33 (m, 1H), 1.22 (d, J = 6.5 Hz, 3H), 1.17 (d, J = 6.5 Hz, |
| | 3H). ESI-MS calculated value C35H46N4O6S2[M + H] ⁺: 682.89, measured value: 682.93 |
| 115-017 | 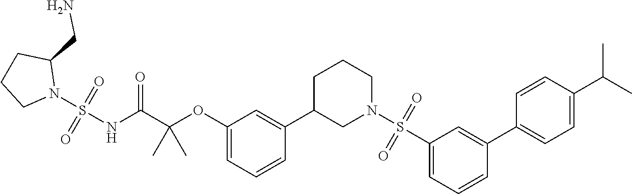 [SMILES: CC(C)c1ccc(-c2cccc(S(=O)(=O)N3CCCC(c4cccc(OC(C)(C)C(=O)NS(=O)(=O)N5CCC[C@H]5CN)c4)C3)c2)cc1] |
| | N-(2-(R)-(aminomethyl)pyrrolidine-1-yl)sulfonyl)-2-(3-(1-(((4′-isopropyl-[1,1′-biphenyls). ]-3-yl) |
| | sulfonylpiperidine-3-yl)phenoxy)-2-methylpropionamide |
| | ^{1H NMR (400 MHz, DMSO-}d6) δ 7.93 (dd, J = 6.9, 1.5 Hz, 2H), 7.83-7.75 (m, 1H), 7.63-7.56 (m, 2H), 7.60- |
| | 7.47 (m, 1H), 7.33 (d, J = 8.1 Hz, 2H), 7.25-7.17 (m, 1H), 7.00 (s, 1H), 6.96-6.88 (m, 1H), 6.79 (t, J = |
| | 2.2 Hz, 1H), 6.74 (dt, J = 7.0, 1.6 Hz, 1H), 4.48 (t, J = 6.6 Hz, 2H), 4.19-4.09 (m, 1H), 4.01-3.91 (m, 1H), |
| | 3.40-3.31 (m, 2H), 3.20 (dd, J = 12.5, 5.1 Hz, 1H), 2.91 (ddd, J = 12.6, 9.1, 5. 9 Hz, 2H), 2.75-2.65 (m, 1H), |
| | 2.51 (tdd, J = 6.8, 5.1, 3.2 Hz, 2H), 2.27 (dtd, J = 10.8, 6.5, 4.4 Hz, 1H), 2.09-1.96 (m, 1H), 1.96-1.84 (m, |
| | 1H), 1.80-1.66 (m, 2H), 1.70-1.53 (m, 9H), 1.47-1.33 (m, 1H), 1.22 (d, J = 6.5 Hz, 3H), 1.17 (d, J = 6.5 |
| | Hz, 3H). ESI-MS calculated value C35H46N4O6S2[M + H] ⁺: 682.89, measured value: 682.88 |
| 115-020 | 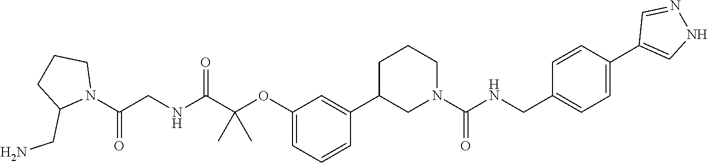 [SMILES: CC(C)(Oc1cccc(C2CCCN(C(=O)NCc3ccc(-c4cn[nH]c4)cc3)C2)c1)C(=O)NCC(=O)N1CCCC1CN] |
| | N-(4-(1H-pyrazole-4-yl)benzyl)-3-(3-(((1-((2-(2-(aminomethyl)pyrrolidin-1-yl)- |
| | 2-Oxyethyl)amino)-2-methyl-1-oxypropionyl)oxy)phenyl) piperidine-1-formamide |
| | ^{1H NMR (400 MHz, DMSO-}d6) δ 8.73 (d, J = 1.8 Hz, 2H), 8.28 (t, J = 5.3 Hz, 1H), 7.88 (t, J = 6.0 Hz, 1H), 7.67-7.59 |
| | (m, 2H), 7.30 (dt, J = 8.4, 1.2 Hz, 2H), 7.25-7.17 (m, 1H), 6.92 (dt, J = 7.9, 1.3 Hz, 1H), 6.79 (d, J = 1.3 Hz, |
| | 1H), 6.74 (dt, J = 7.0, 1.2 Hz, 1H), 4.48 (t, J = 6.8 Hz, 2H), 4.29-4.20 (m, 2H), 4.21 (d, J = 0.9 Hz, 1H), 4.13 |
| | (dt, J = 5.9, 1.0 Hz, 2H), 3.91 (ddd, J = 8.5, 4.7, 3.8 Hz, 1H), 3.83-3.68 (m, 2H), 3.51 (ddd, J = 12.3, 5.1, 3.2 |
| | Hz, 1H), 3.33 (dd, J = 12.3, 4.7 Hz, 1H), 2.76 (ddd, J = 12.4, 6.4, 3.6 Hz, 1H), 2.71-2.60 (m, 2H), 2.47-2.34 |
| | (m, 2H), 2.10 (ddt, J = 11.5, 6.4, 4.7 Hz, 1H), 2.01-1.70 (m, 4H), 1.74-1.62 (m, 1H), 1.62 (s, 3H), 1.57 (s, |
| | 3H), 1.62-1.48 (m, 1H). ESI-MS calculated value C33H43N7O4[M + H] ⁺: 601.75, measured value: 601.23 |
| 115-021 | 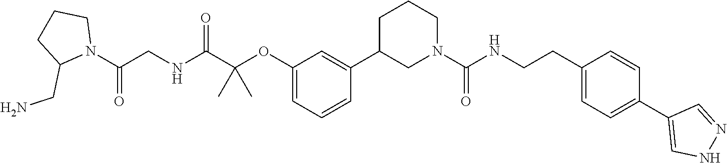 [SMILES: CC(C)(Oc1cccc(C2CCCN(C(=O)NCCc3ccc(-c4cn[nH]c4)cc3)C2)c1)C(=O)NCC(=O)N1CCCC1CN] |
| | N-(4-(1H-pyrazole-4-yl)phenyethyl)-3-(3-(((1-((2-(2-(aminomethyl)pyrrolidine-1-yl)-2-Oxyethyl)Amino)-2- |
| | methyl-1-oxypropionyl) oxy)phenyl) piperidine-1-formamide |
| | ^{1H NMR (400 MHz, DMSO-}d6) δ 8.73 (d, J = 1.8 Hz, 2H), 8.28 (t, J = 5.3 Hz, 1H), 7.64-7.56 (m, 2H), 7.31 (d, J = 8.2 |
| | Hz, 2H), 7.25-7.17 (m, 1H), 6.92 (dd, J = 7.9, 1.3 Hz, 1H), 6.79 (d, J = 1.3 Hz, 1H), 6.74 (dt, J = 7.1, 1.2 Hz, |
| | 1H), 6.22-6.14 (m, 1H), 4.48 (t, J = 6.8 Hz, 2H), 4.29-4.20 (m, 2H), 4.21 (d, J = 0.9 Hz, 1H), 3.91 (ddd, J = |
| | 8.5, 4.7, 3.8 Hz, 1H), 3.83-3.68 (m, 2H), 3.51 (ddd, J = 12.3, 5.1, 3.2 Hz, 1H), 3.33 (dd, J = 12.3, 4.7 Hz, 1H), |
| | 3.11 (s, 2H), 2.76 (ddd, J = 12.5, 6.4, 3.6 Hz, 1H), 2.71-2.60 (m, 2H), 2.47-2.34 (m, 2H), 2.10 (ddt, J = |
| | 11.5, 6.5, 4.7 Hz, 1H), 2.01-1.82 (m, 2H), 1.78 (dtdd, J = 14.0, 6.9, 5.5, 3.5 Hz, 2H), 1.70-1.59 (m, 1H), |
| | 1.62 (s, 3H), 1.57 (s, 3H), 1.61-1.48 (m, 1H). |
| | ESI-MS calculated value C34H45N7O4[M + H] ⁺: 615.77, measured value: 616.35 |
| 115-022 | 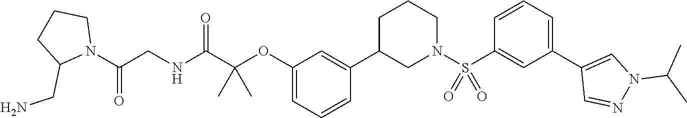 [SMILES: CC(C)n1cc(-c2cccc(S(=O)(=O)N3CCCC(c4cccc(OC(C)(C)C(=O)NCC(=O)N5CCCC5CN)c4)C3)c2)cn1] |
| | N-(2-(2-(aminomethyl)pyrrrolidine-1-yl)-2-oxoethyl)-2-(3-(1-((3-(1-isopropyl-1H-pyrazole) .- 4-yl)phenyl) |
| | sulfonyl)piperidine-3-yl)phenoxy)-2- Methylpropionamide |
| | ^{1H NMR (400 MHz, DMSO-}d6) δ 9.04 (d, J = 1.7 Hz, 1H), 8.32-8.20 (m, 2H), 7.93 (dd, J = 7.0, 1.4 Hz, 2H), 7.79 (dt, |
| | J = 8.8, 1.5 Hz, 1H), 7.56-7.47 (m, 1H), 7.21 (t, J = 7.5 Hz, 1H), 6.92 (dd, J = 8.0, 1.4 Hz, 1H), 6.79 (d, J = 1.3 |
| | Hz, 1H), 6.74 (dt, J = 7.1, 1.2 Hz, 1H), 4.48 (t, J = 6.8 Hz, 2H), 4.34 (p, J = 4.4 Hz, 1H), 4.22 (dd, J = 5.1, 1.0 Hz, |
| | 2H), 3.91 ( ddd, J = 8.5, 4.7, 3.8 Hz, 1H), 3.73 (ddd, J = 12.6, 5.1, 3.4 Hz, 1H), 3.51 (ddd, J = 12.3, 5.1, 3.2 Hz, |
| | 1H), 3.40-3.31 (m, 2H), 3.20 (dd, J = 12.5, 5.1 Hz, 1H), 2.90 (dd, J = 12.5, 5.1 Hz, 1H), 2.65 (dtd, J = 10.6, |
| | 6.8, 3.8 Hz, 1H), 2.50 (q, J = 5.4 Hz, 1H), 2.41 (dddd, J = 11.7, 10.2, 4.2, 3.2 Hz, 2H), 2.10 (ddt, J = 11.5, |
| | 6.4, 4.7 Hz, 1H), 1.96-1.84 (m, 2H), 1.76 (dddd, J = 13.1, 8.2, 4.2, 2.4 Hz, 1H), 1.67-1.53 (m, 8H), 1.46- |
| | 1.33 (m, 1H), 1.32 (d, J = 4.4 Hz, 3H), 1.27 (d, J = 4.4 Hz, 3H). |
| | ESI-MS calculated value C34H46N6O5S[M + H] ⁺: 650.84, measured value: 650.65 |
| 115-023 | 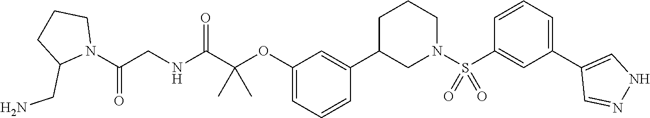 [SMILES: CC(C)(Oc1cccc(C2CCCN(S(=O)(=O)c3cccc(-c4cn[nH]c4)c3)C2)c1)C(=O)NCC(=O)N1CCCC1CN] |
| | 2-(3-(1-((3-(1H-pyrazole-4-yl)phenyl)sulfonyl)piperidine-3-yl).Phenoxy)-N-(2-(2-(aminomethyl)pyrrrolidine- |
| | 1-yl)-2-oxyethyl)-2-methylpropionamide |
| | 1H NMR (400 MHz, DMSO-d6) δ 8.73 (d, J = 1.8 Hz, 2H), 8.28 (t, J = 5.3 Hz, 1H), 7.93 (dd, J = 7.0, 1.4 Hz, |
| | 2H), 7.79 (dt, J = 8.8, 1.5 Hz, 1H), 7.56-7.47 (m, 1H), 7.21 (t, J = 7.5 Hz, 1H), 6.92 (dt, J = 8.0, 1.4 Hz, 1H), |
| | 6.79 (d, J = 1.3 Hz, 1H), 6.74 (dt, J = 7.1, 1.2 Hz, 1H), 4.48 (t, J = 6.8 Hz, 2H), 4.22 (dd, J = 5.1, 1.0 Hz, 2H), |
| | 3.91 (ddd, J = 8.5, 4.7, 3.8 Hz, 1H), 3.73 (ddd, J = 12.5, 5.1, 3.4 Hz, 1H), 3.51 (ddd, J = 12.3, 5.1, 3.2 Hz, |
| | 1H), 3.40-3.31 (m, 2H), 3.20 (dd, J = 12.5, 5.1 Hz, 1H), 2.90 (dd, J = 12.5, 5.1 Hz, 1H), 2.65 (dtd, J = 10.6, |
| | 6.8, 3.8 Hz, 1H), 2.50 (q, J = 5.4 Hz, 1H), 2.46-2.34 (m, 2H), 2.10 (ddt, J = 11.5, 6.4, 4.7 Hz, 1H), 1.98-1.84 |
| | (m, 2H), 1.81-1.69 (m, 1H), 1.67-1.53 (m, 8H), 1.46-1.33 (m, 1H). ESI-MS calculated value |
| | C31H40N6O5S[M + H] +: 609.28, measured value: 609.25 |
| 115-024 | 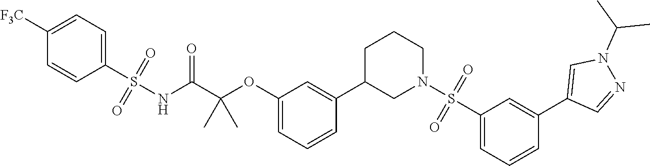 [SMILES: CC(C)n1cc(-c2cccc(S(=O)(=O)N3CCCC(c4cccc(OC(C)(C)C(=O)NS(=O)(=O)c5ccc(C(F)(F)F)cc5)c4)C3)c2)cn1] |
| | 2-(3-(1-(1-(1-isopropyl-1H-pyrazole-4-yl)phenyl)sulfonyl)piperidine- |
| | 3-yl)phenoxy)-2-methyl-N-((4-(trifluoromethyl)phenyl)sulfonyl) Propionamide |
| | 1H NMR (400 MHz, DMSO-d6) δ 8.73 (d, J = 1.8 Hz, 2H), 7.93 (dd, J = 6.9, 1.5 Hz, 2H), 7.83-7.76 (m, 1H), |
| | 7.76-7.69 (m, 2H), 7.57-7.47 (m, 1H), 7.39-7.31 (m, 2H), 7.25-7.17 (m, 1H), 6.96-6.88 (m, 1H), 6.79 |
| | (t, J = 2.2 Hz, 1H), 6.74 (dt, J = 7.0, 1.6 Hz, 1H), 3.40-3.31 (m, 2H), 3.20 (dd, J = 12.5, 5.1 Hz, 1H), 2.90 (dd, |
| | J = 12.5, 5.1 Hz, 1H), 2.51 (p, J = 5.4 Hz, 1H), 1.96-1.84 (m, 1H), 1.67-1.53 (m, 8H), 1.47-1.33 (m, 1H). |
| | ESI-MS Calculated Value C34H37F3N4O6S2 [M + H] +: 719.21 Measured Value: 719.16 |
| 115-025 | 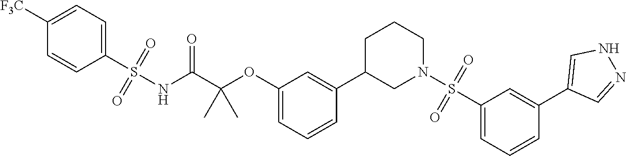 [SMILES: CC(C)(Oc1cccc(C2CCCN(S(=O)(=O)c3cccc(-c4cn[nH]c4)c3)C2)c1)C(=O)NS(=O)(=O)c1ccc(C(F)(F)F)cc1] |
| | 2-(3-(1-((3-(1H-pyrazole-4-yl)phenyl)sulfonyl)piperidine-3-yl).Phenoxy)-2-methyl-N-((4-(trifluoromethyl) |
| | phenyl)sulfonyl)propionamide |
| | 1H NMR (400 MHz, DMSO-d6) δ 8.73 (d, J = 1.8 Hz, 2H), 7.93 (dd, J = 6.9, 1.5 Hz, 2H), 7.83-7.76 (m, 1H), |
| | 7.76-7.69 (m, 2H), 7.57-7.47 (m, 1H), 7.39-7.31 (m, 2H), 7.25-7.17 (m, 1H), 6.96-6.88 (m, 1H), |
| | 6.79 (t, J = 2.2 Hz, 1H), 6.74 (dt, J = 7.0, 1.6 Hz, 1H), 3.40-3.31 (m, 2H), 3.20 (dd, J = 12.5, 5.1 Hz, 1H), 2.90 |
| | (dd, J = 12.5, 5.1 Hz, 1H), 2.51 (p, J = 5.4 Hz, 1H), 1.96-1.84 (m, 1H), 1.67-1.53 (m, 8H), 1.47-1.33 (m, |
| | 1H). |
| | ESI-MS calculated value C31H31F3N4O6S2[M + H] +: 677.18, measured value: 677.15 |
| 115-026 | 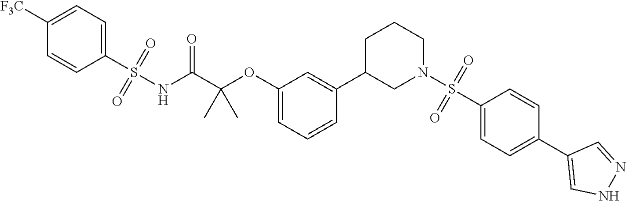 [SMILES: CC(C)(Oc1cccc(C2CCCN(S(=O)(=O)c3ccc(-c4cn[nH]c4)cc3)C2)c1)C(=O)NS(=O)(=O)c1ccc(C(F)(F)F)cc1] |
| | 2-(3-(1-((4-(1H-pyrazole-4-yl)phenyl)sulfonyl)piperidine-3-yl).Phenoxy)-2-methyl-N-((4-(trifluoromethyl) |
| | phenyl)sulfonyl)propionamide |
| | 1H NMR (400 MHz, DMSO-d6) δ 8.73 (d, J = 1.8 Hz, 2H), 7.92-7.84 (m, 2H), 7.77-7.69 (m, 2H), 7.69- |
| | 7.61 (m, 2H), 7.39-7.31 (m, 2H), 7.25-7.17 (m, 1H), 6.96-6.88 (m, 1H), 6.79 (t, J = 2.2 Hz, 1H), 6. 74 (dt, |
| | J = 7.0, 1.6 Hz, 1H), 3.40-3.31 (m, 2H), 3.20 (dd, J = 12.5, 5.1 Hz, 1H), 2.90 (dd, J = 12.5, 5.1 Hz, 1H), 2.51 |
| | (p, J = 5.4 Hz, 1H), 1.96-1.84 (m, 1H), 1.67-1.53 (m, 8H), 1.47-1.33 (m, 1H). |
| | ESI-MS calculated value C31H31F3N4O6S2[M + H] +: 677.18, measured value: 677.34 |
| 115-027 | 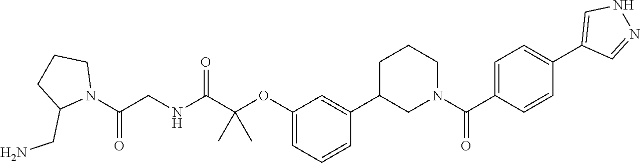 [SMILES: CC(C)(Oc1cccc(C2CCCN(C(=O)c3ccc(-c4cn[nH]c4)cc3)C2)c1)C(=O)NCC(=O)N1CCCC1CN] |
| | 2-(3-(1-(4-(1H-pyrazole-4-yl)benzoyl)piperidine-3-yl)phenoxy)-N-(2-(2-(Aminomethyl)pyrrrolidine-1-yl)-2- |
| | oxoethyl)-2-methylpropionamide |
| | 1H NMR (400 MHz, DMSO-d6) δ 8.73 (d, J = 1.8 Hz, 2H), 8.28 (t, J = 5.3 Hz, 1H), 8.09-8.01 (m, 2H), 7.93- |
| | 7.85 (m, 2H), 7.21 (t, J = 7.5 Hz, 1H), 6.92 (dt, J = 8.1, 1.4 Hz, 1H), 6.79 (d, J = 1.3 Hz, 1H), 6.74 (dt, J = 7.0, |
| | 1.2 Hz, 1H), 4.48 (t, J = 6.8 Hz, 2H), 4.29-4.20 (m, 2H), 4.21 (d, J = 0.9 Hz, 1H), 3.91 (ddd, J = 8.5, 4.7, 3.8 |
| | Hz, 1H), 3.73 (ddd, J = 12.5, 5.1, 3.4 Hz, 1H), 3.51 (ddd, J = 12.3, 5.1, 3.2 Hz, 1H), 3.32 (ddd, J = 12.5, 6.1, |
| | 4.0 Hz, 2H), 3.22 (ddd, J = 12.5, 6.1, 3.5 Hz, 1H), 2.65 (ddt, J = 9.9, 7.1, 3.6 Hz, 2H), 2.47-2.34 (m, 2H), 2.10 |
| | (ddt, J = 11.5, 6.4, 4.7 Hz, 1H), 2.01-1.82 (m, 2H), 1.86-1.70 (m, 2H), 1.70-1.59 (m, 1H), 1.62 (s, 3H), |
| | 1.57 (s, 3H), 1.61-1.48 (m, 1H). ESI-MS calculated value C32H40N6O4[M + H] +: 573.71, measured value: |
| | 573.57 |
| 115-028 | 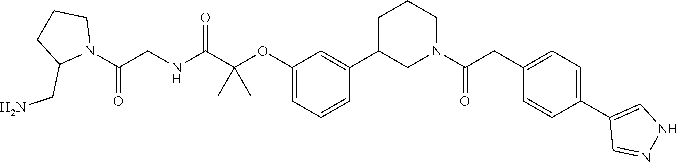 [SMILES: CC(C)(Oc1cccc(C2CCCN(C(=O)Cc3ccc(-c4cn[nH]c4)cc3)C2)c1)C(=O)NCC(=O)N1CCCC1CN] |
| | 2-(3-(1-(2-(4-(4-(1H-pyrazole-4-yl)phenyl)acetyl)piperidine-3-ylPhenoxy)-N-(2-(2-(aminomethyl) |
| | pyrrrolidine-1-yl)-2-oxoethyl)-2-methylpropionamide |
| | 1H NMR (400 MHz, DMSO-d6) δ 8.73 (d, J = 1.8 Hz, 2H), 8.28 (t, J = 5.3 Hz, 1H), 7.64-7.56 (m, 2H), 7.33 |
| | (dt, J = 8.4, 1.2 Hz, 2H), 7.21 (t, J = 7.5 Hz, 1H), 6.92 (dt, J = 7.9, 1.3 Hz, 1H), 6.79 (d, J = 1.2 Hz, 1H), 6.74 (dt, |
| | J = 7.0, 1.2 Hz, 1H), 4.48 (t, J = 6.8 Hz, 2H), 4.25-4.15 (m, 3H), 3.91 (ddd, J = 8.5, 4.7, 3.8 Hz, 1H), 3.83- |
| | 3.67 (m, 3H), 3.56-3.46 (m, 2H), 2.76 (ddd, J = 12.3, 6.3, 3.5 Hz, 1H), 2.71-2.60 (m, 2H), 2.47-2.34 (m, |
| | 2H), 2.10 (ddt, J = 11.5, 6.4, 4.7 Hz, 1H), 2.01-1.85 (m, 2H), 1.89-1.76 (m, 1H), 1.80-1.72 (m, 1H), 1.76- |
| | 1.48 (m, 9H). |
| | ESI-MS calculated value C33H42N6O4[M + H] +: 587.33, measured value: 587.27 |
| 115-029 | 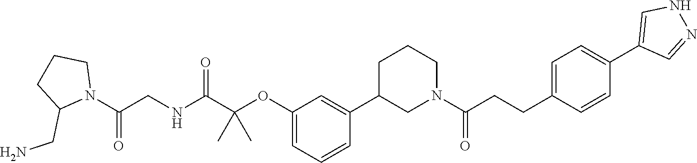 [SMILES: CC(C)(Oc1cccc(C2CCCN(C(=O)CCc3ccc(-c4cn[nH]c4)cc3)C2)c1)C(=O)NCC(=O)N1CCCC1CN] |
| | 2-(3-(1-(3-(4-(1H-pyrazole-4-yl)phenyl)propionyl)piperidine-3-ylPhenoxy)-N-(2-(2-(aminomethyl)pyrrrolidine- |
| | 1-yl)-2-oxoethyl)-2-methylpropionamide |
| | 1H NMR (400 MHz, DMSO-d6) δ 8.73 (d, J = 1.8 Hz, 2H), 8.28 (t, J = 5.3 Hz, 1H), 7.64-7.56 (m, 2H), 7.37- |
| | 7.30 (m, 2H), 7.21 (t, J = 7.5 Hz, 1H), 6.92 (dd, J = 7.9, 1.3 Hz, 1H), 6.79 (d, J = 1.3 Hz, 1H), 6. 74 (dt, J = 7.0, |
| | 1.2 Hz, 1H), 4.48 (t, J = 6.8 Hz, 2H), 4.25-4.15 (m, 3H), 3.91 (ddd, J = 8.5, 4.7, 3.8 Hz, 1H), 3.83-3.68 (m, |
| | 2H), 3.56-3.46 (m, 2H), 2.89 (dd, J = 8.4, 7.2 Hz, 2H), 2.76 (ddd, J = 12.3, 6.3, 3.5 Hz, 1H), 2.71-2.60 (m, |
| | 4H), 2.47-2.34 (m, 2H), 2.10 (ddt, J = 11.5, 6.5, 4.7 Hz, 1H), 2.01-1.85 (m, 2H), 1.89-1.76 (m, 1H), 1.80- |
| | 1.72 (m, 1H), 1.76-1.48 (m, 9H). |
| | ESI-MS calculated value C34H44N6O4[M + H] +: 601.35, measured value: 601.39 |
| 115-030 | 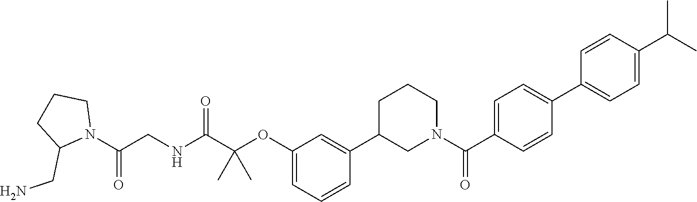 [SMILES: CC(C)c1ccc(-c2ccc(C(=O)N3CCCC(c4cccc(OC(C)(C)C(=O)NCC(=O)N5CCCC5CN)c4)C3)cc2)cc1] |
| | 2-(3-(1-(4-(4′-isopropyl-[1,1′-biphenyl-4-yl)benzoyl)piperidine-3-yl).″Phenoxy)-N-(2-(2-(aminomethyl) |
| | pyrrrolidine-1-yl)-2-oxyethyl)-2-methylpropionamide |
| | ^{1H NMR (400 MHz, DMSO-d6}) δ 8.28 (t, J = 5.3 Hz, 1H), 8.09-8.01 (m, 2H), 7.87-7.79 (m, 2H), 7.63-7.55 (m, 2H), |
| | 7.33 (d, J = 8.1 Hz, 2H), 7.25-7.17 (m, 1H), 6.92 (dd, J = 8.0, 1.4 Hz, 1H), 6.79 (d, J = 1.3 Hz, 1H), 6.74 (dt, J = |
| | 7.1, 1.2 Hz, 1H), 4.48 (t, J = 6.8 Hz, 2H), 4.29-4.20 (m, 2H), 4.21 (d, J = 0.9 Hz, 1H), 3.91 (ddd, J = 8.5, 4.7, |
| | 3.8 Hz, 1H), 3.73 (ddd, J = 12.6, 5.1, 3.4 Hz, 1H), 3.51 (ddd, J = 12.3, 5.1, 3.2 Hz, 1H), 3.32 (ddd, J = 12.5, 6.1, |
| | 4.0 Hz, 2H), 3.22 (ddd, J = 12.6, 6.1, 3.5 Hz, 1H), 2.93 (hept, J = 6.3 Hz, 1H), 2.66 (ddd, J = 10.9, 6.5, 3.9 Hz, |
| | 2H), 2.41 (dddd, J = 11.8, 10.2, 4.2, 3.2 Hz, 2H), 2.10 (ddt, J = 11.6, 6.4, 4.7 Hz, 1H), 2.01-1.82 (m, 2H), |
| | 1.86-1.75 (m, 1H), 1.80-1.67 (m, 1H), 1.70-1.59 (m, 1H), 1.62 (s, 3H), 1.57 (s, 3H), 1.61-1.48 (m, 1H), |
| | 1.22 (d, J = 6.5 Hz, 3H), 1.17 (d, J = 6.5 Hz, 3H). ESI-MS calculated value C38H48N4O4[M + H] ⁺: 625.83, |
| | measured value: 625.89 |
| 115-031 | 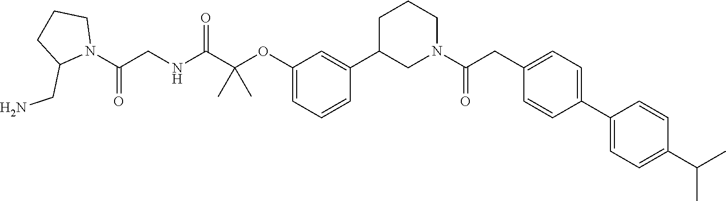 [SMILES: CC(C)c1ccc(-c2ccc(CC(=O)N3CCCC(c4cccc(OC(C)(C)C(=O)NCC(=O)N5CCCC5CN)c4)C3)cc2)cc1] |
| | N-(2-(2-(aminomethyl)pyrrolidin-1-yl)-2-oxoethyl)-2-(3-(1-(2-(4′-isopropyl-[1,1′-] |
| | Biphenyl]-4-yl)acetyl)piperidine-3-yl)phenoxy)-2-Methylpropionamide |
| | 1H NMR (400 MHz, DMSO-d6) δ 8.28 (t, J = 5.3 Hz, 1H), 7.63-7.55 (m, 4H), 7.33 (dd, J = 8.4, 1.7 Hz, 4H), |
| | 7.25-7.17 (m, 1H), 6.92 (dd, J = 7.9, 1.3 Hz, 1H), 6.79 (d, J = 1.3 Hz, 1H), 6.74 (dt, J = 7.1, 1.2 Hz, 1H), 4.48 |
| | (t, J = 6.8 Hz, 2H), 4.25-4.15 (m, 3H), 3.91 (ddd, J = 8.5, 4.7, 3.8 Hz, 1H), 3.83-3.67 (m, 3H), 3.56-3.46 |
| | (m, 2H), 2.93 (p, J = 6.8 Hz, 1H), 2.76 (ddd, J = 12.3, 6.3, 3.5 Hz, 1H), 2.71-2.60 (m, 2H), 2.47-2.34 (m, |
| | 2H), 2.10 (ddt, J = 11.6, 6.5, 4.7 Hz, 1H), 2.01-1.85 (m, 2H), 1.89-1.76 (m, 1H), 1.77 (ddd, J = 8.9, 4.2, 2.3 |
| | Hz, 1H), 1.76-1.48 (m, 9H), 1.22 (d, J = 6.5 Hz, 3H), 1.17 (d, J = 6.5 Hz, 3H). |
| | ESI-MS calculated value C39H50N4O4[M + H] +: 639.38, measured value: 639.49 |
| 115-047 | 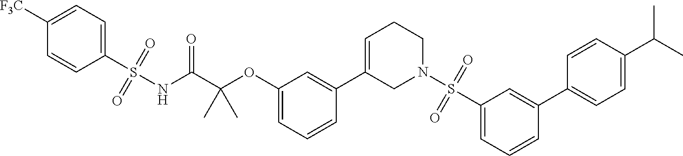 [SMILES: CC(C)c1ccc(-c2cccc(S(=O)(=O)N3CCC=C(c4cccc(OC(C)(C)C(=O)NS(=O)(=O)c5ccc(C(F)(F)F)cc5)c4)C3)c2)cc1] |
| | 2-(3-(1-(((4′-isopropyl-[1,1′-biphenyl]-3-yl)sulfonyl)-1,2,5,6-tetrahydropyridine-3-ylPhenoxy)-2-methyl-N- |
| | ((4-(trifluoromethyl)phenyl)sulfonyl)propionamide 1H NMR ( 400 MHz, DMSO-d6) δ 7.93 (dd, J = 6.9, 1.5 Hz, |
| | 2H), 7.83-7.76 (m, 1H), 7.76-7.69 (m, 2H), 7.63-7.56 (m, 2H), 7.56-7.48 (m, 1H), 7.45 (t, J = 7.7 Hz, |
| | 1H), 7.39-7.29 (m, 4H), 7.06 (t, J = 1.8 Hz, 1H), 6.81 (dt, J = 7.4, 1.4 Hz, 1H), 6.73 (dt, J = 7.9, 1.2 Hz, 1H), |
| | 6.08 (td, J = 4.1, 1.1 Hz, 1H), 3.33 (s, 1H), 3.00 (t, J = 5.2 Hz, 2H), 2.93 (p, J = 6.8 Hz, 1H), 2.30 (dtd, J = 6.5, |
| | 5.4, 4.9, 1.3 Hz, 2H), 1.60 (s, 5H), 1.20 (d, J = 6.5 Hz, 6H). ESI-MS calculated value C37H37F3N2O6S2 [M + H] ⁺: |
| | 727.75, measured value: 727.53 |
| 115-049 | 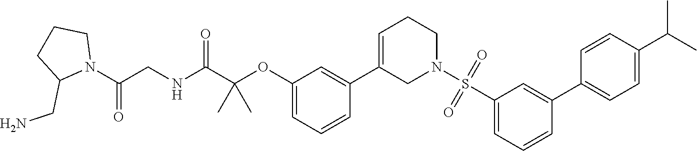 [SMILES: CC(C)c1ccc(-c2cccc(S(=O)(=O)N3CCC=C(c4cccc(OC(C)(C)C(=O)NCC(=O)N5CCCC5CN)c4)C3)c2)cc1] |
| | N-(2-(2-(aminomethyl)pyrrrolidine-1-yl)-2-oxoethyl)-2-(3-(1-((4′-isopropyl-[1,1′-biphenyls.).″ ]-3-yl)sulfonyl)- |
| | 1,2,5,6-tetrahydropyridine-3-yl)phenoxy)-2-Methylpropionamide |
| | 1H NMR (400 MHz, DMSO-d6) δ 8.28 (t, J = 5.3 Hz, 1H), 7.93 (dd, J = 6.9, 1.5 Hz, 2H), 7.83-7.75 (m, 1H), |
| | 7.63-7.56 (m, 2H), 7.56-7.48 (m, 1H), 7.45 (t, J = 7.7 Hz, 1H), 7.33 (d, J = 8.1 Hz, 2H), 7.06 (t, J = 1.8 Hz, |
| | 1H), 6.81 (dt, J = 7.4, 1.5 Hz, 1H), 6.73 (dt, J = 7.9, 1.2 Hz, 1H), 6.08 (td, J = 4.1, 1.1 Hz, 1H), 4.48 (t, J = 6.8 |
| | Hz, 2H), 4.22 (dd, J = 5.1, 1.0 Hz, 2H), 3.91 (ddd, J = 8.5, 4.7, 3.8 Hz, 1H), 3.73 (ddd, J = 12.6, 5.1, 3.4 Hz, |
| | 1H), 3.51 (ddd, J = 12.3, 5.1, 3.2 Hz, 1H), 3.00 (t, J = 5.2 Hz, 2H), 2.93 (p, J = 6.8 Hz, 1H), 2.65 (dtd, J = 10.6, |
| | 6.8, 3.8 Hz, 1H), 2.46-2.34 (m, 2H), 2.30 (dtd, J = 6.5, 5.4, 4.9, 1.3 Hz, 2H), 2.10 (ddt, J = 11.6, 6.4, 4.7 Hz, |
| | 1H), 1.96-1.84 (m, 1H), 1.76 (dddt, J = 11.8, 10.0, 8.2, 4.0 Hz, 1H), 1.62 (s, 3H), 1.57 (s, 3H), 1.22 (d, J = 6.5 |
| | Hz, 3H), 1.17 (d, J = 6.5 Hz, 3H). The calculated value of ESI-MS C37H46N4O5S[M + H] +: 659.32, the |
| | measured value: 659.37 |
| 115-052 | 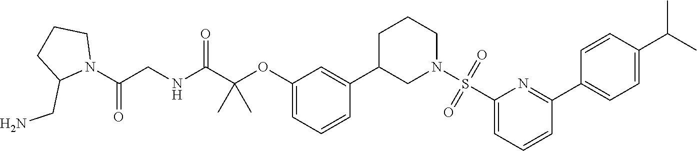 [SMILES: CC(C)c1ccc(-c2cccc(S(=O)(=O)N3CCCC(c4cccc(OC(C)(C)C(=O)NCC(=O)N5CCCC5CN)c4)C3)n2)cc1] |
| | N-(2-(2-(aminomethyl)pyrrrolidine-1-yl)-2-oxoethyl)-2-(3-(1-((6-(4-isopropylphenyl)pyridine-2-yl)sulfonyl) |
| | piperidine-3-yl)phenoxy)-2-methylpropionamide |
| | 1H NMR (400 MHz, DMSO-d6) δ 8.28 (t, J = 5.3 Hz, 1H), 8.17-8.09 (m, 2H), 7.88-7.79 (m, 2H), 7.66- |
| | 7.57 (m, 1H), 7.37-7.29 (m, 2H), 7.25-7.17 (m, 1H), 6.96-6.88 (m, 1H), 6.79 (t, J = 2.2 Hz, 1H), 6.74 |
| | (dt, J = 7.0, 1.6 Hz, 1H), 4.48 (t, J = 6.8 Hz, 2H), 4.22 (dd, J = 5.1, 1.0 Hz, 2H), 3.91 (ddd, J = 8.5, 4.7, 3.8 Hz, |
| | 1H), 3.73 (ddd, J = 12.6, 5.1, 3.4 Hz, 1H), 3.51 (ddd, J = 12.3, 5.1, 3.2 Hz, 1H), 3. 40-3.31 (m, 2H), 3.20 (dd, |
| | J = 12.5, 5.1 Hz, 1H), 2.91 (ddd, J = 12.6, 9.1, 5.9 Hz, 2H), 2.65 (dtd, J = 10.6, 6.8, 3.8 Hz, 1H), 2.50 (q, J = 5.4 |
| | Hz, 1H), 2.46-2.34 (m, 2H), 2.10 (ddt, J = 11.5, 6.4, 4.7 Hz, 1H), 1.96-1.82 (m, 2H), 1.82-1.69 (m, 1H), |
| | 1.67-1.53 (m, 8H), 1.47-1.33 (m, 1H), 1.22 (d, J = 6.5 Hz, 3H), 1.17 (d, J = 6.5 Hz, 3H). ESI-MS calculated |
| | value C36H47N5O5S[M + H] +: 662.33, measured value: 662.39 |
| 115-055 | 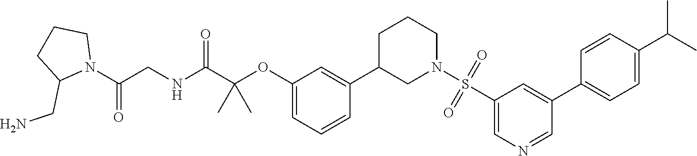 [SMILES: CC(C)c1ccc(-c2cncc(S(=O)(=O)N3CCCC(c4cccc(OC(C)(C)C(=O)NCC(=O)N5CCCC5CN)c4)C3)c2)cc1] |
| | N-(2-(2-(aminomethyl)pyrrrolidine-1-yl)-2-oxoethyl)-2-(3-(1-((5-(4-isopropylphenyl)pyridine-3-yl)sulfonyl) |
| | piperidine-3-yl)phenoxy)-2-Methylpropionamide |
| | 1H NMR (400 MHz, DMSO-d6) δ 8.28 (t, J = 5.3 Hz, 1H), 8.17-8.09 (m, 2H), 7.88-7.79 (m, 2H), 7.66- |
| | 7.57 (m, 1H), 7.37-7.29 (m, 2H), 7.25-7.17 (m, 1H), 6.96-6.88 (m, 1H), 6.79 (t, J = 2.2 Hz, 1H), 6.74 |
| | (dt, J = 7.0, 1.6 Hz, 1H), 4.48 (t, J = 6.8 Hz, 2H), 4.22 (dd, J = 5.1, 1.0 Hz, 2H), 3.91 (ddd, J = 8.5, 4.7, 3.8 Hz, |
| | 1H), 3.73 (ddd, J = 12.6, 5.1, 3.4 Hz, 1H), 3.51 (ddd, J = 12.3, 5.1, 3.2 Hz, 1H), 3. 40-3.31 (m, 2H), 3.20 (dd, |
| | J = 12.5, 5.1 Hz, 1H), 2.91 (ddd, J = 12.6, 9.1, 5.9 Hz, 2H), 2.65 (dtd, J = 10.6, 6.8, 3.8 Hz, 1H), 2.50 (q, J = 5.4 |
| | Hz, 1H), 2.46-2.34 (m, 2H), 2.10 (ddt, J = 11.5, 6.4, 4.7 Hz, 1H), 1.96-1.82 (m, 2H), 1.82-1.69 (m, 1H), |
| | 1.67-1.53 (m, 8H), 1.47-1.33 (m, 1H), 1.22 (d, J = 6.5 Hz, 3H), 1.17 (d, J = 6.5 Hz, 3H). ESI-MS calculated |
| | value C36H47N5O5S[M + H] +: 662.33, measured value: 662.45 |

### Test Embodiment

### I Test Method

### 1) CCK8

1. SW480 Cell plate
Cells digested, counted, and laid a 96-well plate (flat-bottomed transparent) with 10,000 cells/100 ul DMEM (10% FBS) per well;
2. Observe the cell status on the next day, and start adding medicine after the cells are intact;
3. Dilute the compound with DMEM (2% FBS) and dilute it in a gradient at concentrations of 20 μM, 10 μM, 5 μM, 2.5 μM, 1.25 μM, 0.625 μM, 0.3125 μM, 0.15625 μM, 0 μM (equal volume DMSO);
4. Add 100 μl of the above compounds of different concentrations to each well, and make 2 compound wells for each concentration gradient; Leave 3 blank wells (DMEM 2% FBS culture medium only, no cells);
5. Incubate at 37 °C for 24 hours;
6. Add 10 μl of CCK-8 enhanced solution to each well: Since the amount of CCK-8 added to each well is relatively small, it may cause errors due to the reagent getting on the well wall, so it is recommended to gently tap the culture plate after adding the reagent to help mix well;
7. Incubate in the incubator for 0.5-4 hours: Different cell types have different amounts of formamin, and 1 hour of incubation is sufficient for most cases. If the color development is not sufficient, the culture can be continued to confirm the optimal conditions.
8. Measure absorbance at 450 nm and absorbance at 600 nm (exclude interference from the reagent base color and the absorbance value of the plate itself);
9. The final absorbance value is OD450 nm-OD600 nm, and the rejection rate is calculated.

Suppression Rate=[(Ac−As)/(Ac−Ab)]×100%
As: absorbance of the experimental wells (medium containing cells, CCK-8, drug to be tested);
Ac: absorbance of the control wells (medium containing cells, CCK-8, no drug to be tested);
Ab: Absorbance of blank wells (medium without cells and drug to be tested, CCK-8).

### (2) SRP Test

1. Experimental settings
1. The analyte is in powder form.
2. The temperature is 25 °C
3. The analysis equipment adopts Biacore T200 instrument.
2. Sample dilution
1. Ligand β-catenin diluted with HEPES (pH 7.4), 0.5 mg/mL
2. The analyte was dissolved in DMSO and diluted with 0.1% DMSO HEPES (pH 7.4).
3. Experimental process
1. Operate and start according to the standard of Biacore T200 instrument.
2. Prepare buffer HEPES (pH 7.4) and 500 ml of deionized water for washing syringes (filtered by 0.22 m membrane).
3. Start installing the chip and install the Cm5 chip according to the standard process.
4. Prepare to start the formal experiment, the buffer will flush the flow path system inside the entire system at a high flow rate.
5. Choose the appropriate program according to the sample size.
6. Start capturing the chip and prepare a sufficient volume of ligands β-catenin, EDC/NHS, and blocking solution. The conjugation procedure was started with a conjugation time of 7 min, a flow rate of 10 l/min, and a final ligand conjugation of approximately 16000RU.
7. After the conjugation is completed, the sample detection is started, and the analyte binding time is set to 120 s.
Flow rate of 30 μL/min; The dissociation time was 200 s and the flow rate was 30 μL/min. Regeneration time 30 s,
The flow rate is 30 μL/min.
8. Prepare the corresponding samples that need to be tested according to the requirements, and start the automatic operation program for testing.
9. Result analysis, according to the operation results, the fitting analysis of the data is carried out to obtain the final affinity fitting KD value.

### (3) Test Results

The test results are shown in Table 1

TABLE 1

| Compound | |
|---|---|
| number | structure |
| 115-010 | 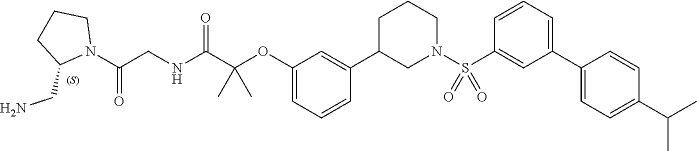 |
| 115-011 | 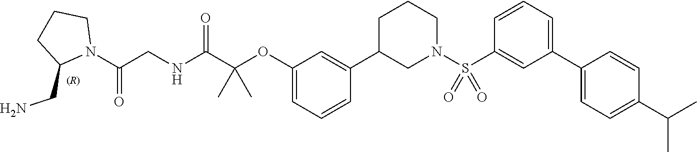 |
| 115-012 | 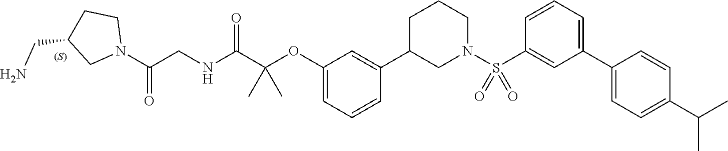 |
| 115-013 | 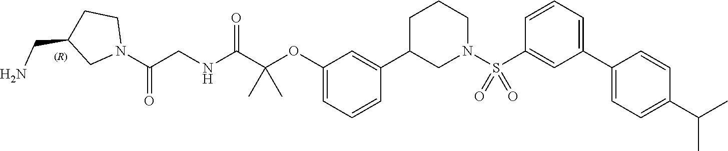 |
| 115-014 | 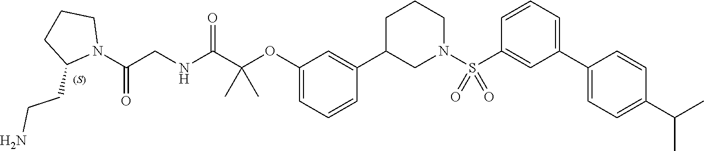 |
| 115-017 | 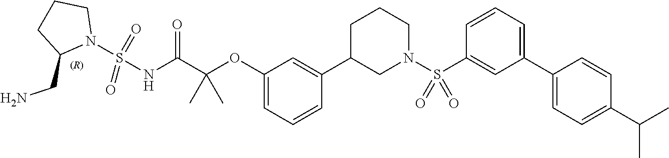 |
| 115-020 | 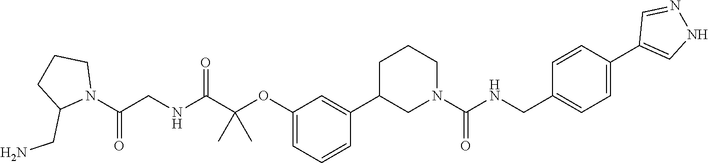 [SMILES: CC(C)(Oc1cccc(C2CCCN(C(=O)NCc3ccc(-c4cn[nH]c4)cc3)C2)c1)C(=O)NCC(=O)N1CCCC1CN] |
| 115-021 | 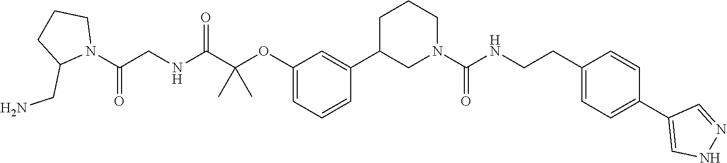 [SMILES: CC(C)(Oc1cccc(C2CCCN(C(=O)NCCc3ccc(-c4cn[nH]c4)cc3)C2)c1)C(=O)NCC(=O)N1CCCC1CN] |
| 115-027 | 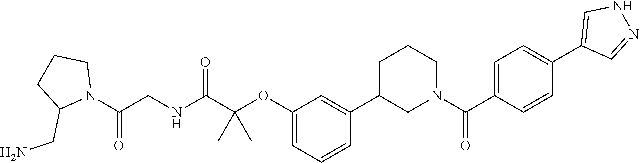 [SMILES: CC(C)(Oc1cccc(C2CCCN(C(=O)c3ccc(-c4cn[nH]c4)cc3)C2)c1)C(=O)NCC(=O)N1CCCC1CN] |
| 115-028 | 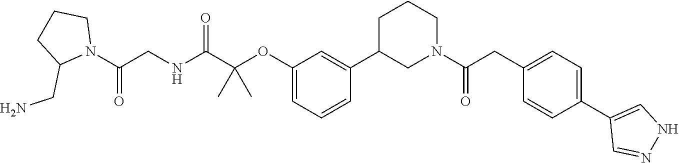 [SMILES: CC(C)(Oc1cccc(C2CCCN(C(=O)Cc3ccc(-c4cn[nH]c4)cc3)C2)c1)C(=O)NCC(=O)N1CCCC1CN] |
| 115-029 | 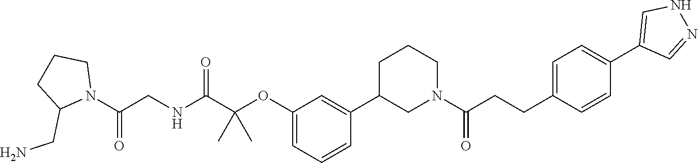 [SMILES: CC(C)(Oc1cccc(C2CCCN(C(=O)CCc3ccc(-c4cn[nH]c4)cc3)C2)c1)C(=O)NCC(=O)N1CCCC1CN] |
| 115-030 | 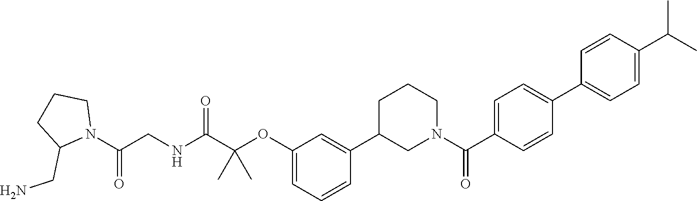 [SMILES: CC(C)c1ccc(-c2ccc(C(=O)N3CCCC(c4cccc(OC(C)(C)C(=O)NCC(=O)N5CCCC5CN)c4)C3)cc2)cc1] |
| 115-031 | 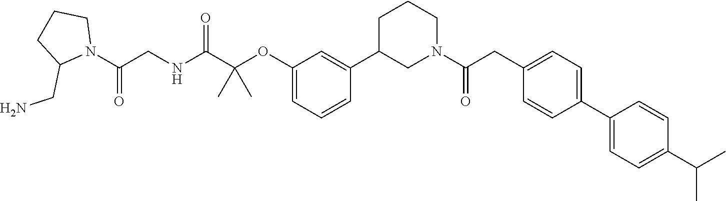 [SMILES: CC(C)c1ccc(-c2ccc(CC(=O)N3CCCC(c4cccc(OC(C)(C)C(=O)NCC(=O)N5CCCC5CN)c4)C3)cc2)cc1] |
| 115-022 | 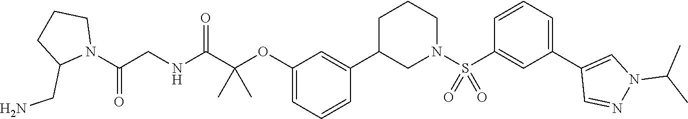 [SMILES: CC(C)n1cc(-c2cccc(S(=O)(=O)N3CCCC(c4cccc(OC(C)(C)C(=O)NCC(=O)N5CCCC5CN)c4)C3)c2)cn1] |
| 115-023 | 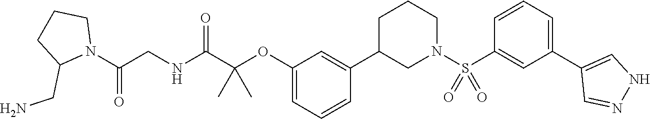 [SMILES: CC(C)(Oc1cccc(C2CCCN(S(=O)(=O)c3cccc(-c4cn[nH]c4)c3)C2)c1)C(=O)NCC(=O)N1CCCC1CN] |
| 115-049 | 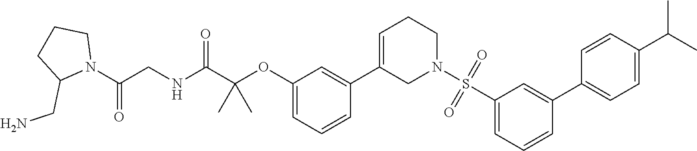 [SMILES: CC(C)c1ccc(-c2cccc(S(=O)(=O)N3CCC=C(c4cccc(OC(C)(C)C(=O)NCC(=O)N5CCCC5CN)c4)C3)c2)cc1] |
| 115-025 | 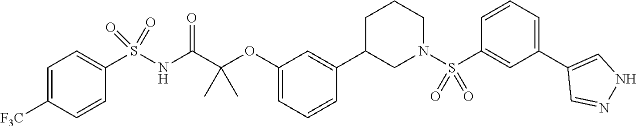 [SMILES: CC(C)(Oc1cccc(C2CCCN(S(=O)(=O)c3cccc(-c4cn[nH]c4)c3)C2)c1)C(=O)NS(=O)(=O)c1ccc(C(F)(F)F)cc1] |
| 115-024 | 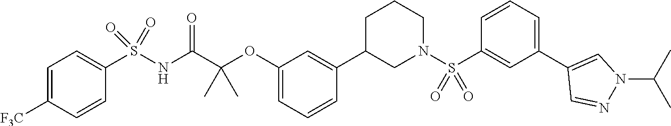 [SMILES: CC(C)n1cc(-c2cccc(S(=O)(=O)N3CCCC(c4cccc(OC(C)(C)C(=O)NS(=O)(=O)c5ccc(C(F)(F)F)cc5)c4)C3)c2)cn1] |
| 115-026 | 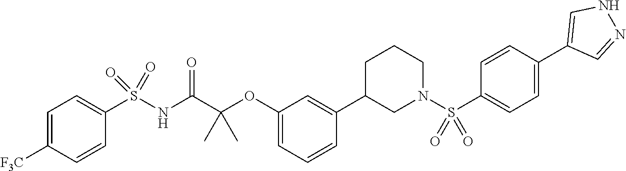 [SMILES: CC(C)(Oc1cccc(C2CCCN(S(=O)(=O)c3ccc(-c4cn[nH]c4)cc3)C2)c1)C(=O)NS(=O)(=O)c1ccc(C(F)(F)F)cc1] |
| 115-052 | 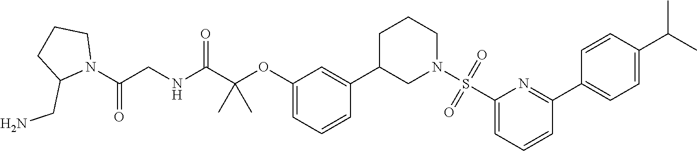 [SMILES: CC(C)c1ccc(-c2cccc(S(=O)(=O)N3CCCC(c4cccc(OC(C)(C)C(=O)NCC(=O)N5CCCC5CN)c4)C3)n2)cc1] |
| 115-055 | 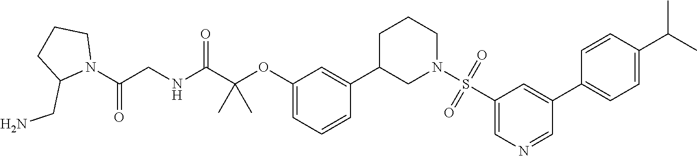 [SMILES: CC(C)c1ccc(-c2cncc(S(=O)(=O)N3CCCC(c4cccc(OC(C)(C)C(=O)NCC(=O)N5CCCC5CN)c4)C3)c2)cc1] |
| 115 | 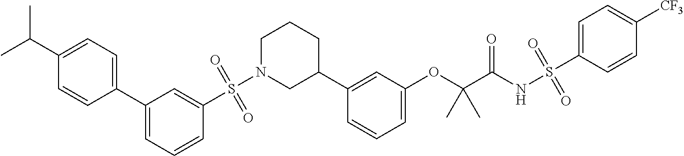 [SMILES: CC(C)c1ccc(-c2cccc(S(=O)(=O)N3CCCC(c4cccc(OC(C)(C)C(=O)NS(=O)(=O)c5ccc(C(F)(F)F)cc5)c4)C3)c2)cc1] |

| Compound | | |
|---|---|---|
| number | SW¹0 IC50(μM) | Kd(μM) |
| 115-010 | 3.5599 μM | 284 μM |
| 115-011 | 6.25 μM~3.125 μM | / |
| 115-012 | <2.972 μM | 760 μM |
| 115-013 | 3.124 μM | |
| 115-014 | 52.88 μM | |
| 115-017 | 10.61 μM | |
| 115-020 | Inactive | |
| 115-021 | Inactive | |
| 115-027 | 23.07 μM | |
| 115-028 | 15.73 μM | |
| 115-029 | 20.84 μM | |
| 115-030 | 6.25 μM~3.125 μM | |
| 115-031 | 6.994 μM | |
| 115-022 | 9.708 μM | |
| 115-023 | 30.54 μM | |
| 115-049 | < 2.646 μM | |
| 115-025 | Inactive | |
| 115-024 | 11.1 μM | |
| 115-026 | Inactive | |
| 115-052 | ≤2.742 μM | |
| 115-055 | 4.060 μM | |
| 115 | 2.812 μM | |

All documents referred to in the present invention are cited as references in the present application, as if each document were cited separately as references. In addition, it should be understood that after reading the above lecture content of the present invention, a person skilled in the field may make various changes or modifications to the present invention, and these equivalent forms also fall within the scope of the claims attached to the present application.

1. A compound or a pharmaceutically acceptable salt thereof, or an isomer, a solvate, a crystal form, or a prodrug thereof, wherein
the compound is of Formula I 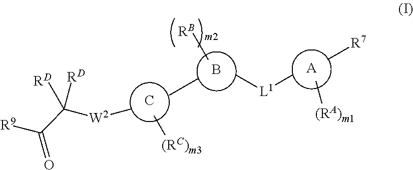
wherein
R⁹ is 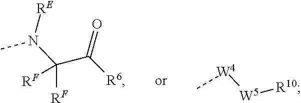
W⁴ and W⁵ are each independently selected from the group consisting of: null, —O—, —S—, —C(O)—, —S(O)—, —S(O)₂—, —N(R^{s1})—, —C(R^{s2})₂—;
R¹⁰ is H, OH, R⁶, or a group that is unsubstituted or substituted with one or more R^{H} groups selected from the group consisting of: C₁₋₆ alkyl groups, C₃₋₁₀ naphthenic alkyl groups, 4 to 10 members heterocyclic alkyl groups, C₃₋₁₀ cyclic alkyl groups, 4 to 10 members heterocyclic alkyl groups, C₆₋₁₀ aryl groups, and 5 to 10 members heteroaryl groups;
R^{H} is selected from the following group: R, optionally substituted C₁₋₆ halogenated alkyl groups;
R^{E} is selected from the group consisting of: H, optionally substituted C₁₋₄ alkyl groups, optionally substituted C₃₋₁₀ naphthenic alkyl groups, optionally substituted 4 to 10 membered heteronaphthenic alkyl groups;
R^{F} is each independently selected from the group consisting of: H, optionally substituted C₁₋₄ alkyl groups, optionally substituted C₃₋₁₀ naphthenic alkyl groups, optionally substituted 4 to 10 membered heteronaphthenic alkyl groups; or two R^{F} and carbon atoms connected to them together form an optionally substituted C₃₋₆ naphthenic group or an optionally substituted 4 to 6-membered heterocyclic group;
R⁶ is optionally substituted groups selected from the group consisting of: —OR², C₃₋₁₂ naphthenic groups, 4 to 10-membered heterocyclic alkyl groups connected to the rest by carbon atoms on the ring, and —NR⁴R⁵;
R² is selected from the group consisting of: H, optionally substituted C₁₋₆ alkyl group, optionally substituted C₃₋₁₀ naphthenic alkyl group, optionally substituted 4 to 8-membered heteronaphnolyl group, optionally substituted C₆₋₁₀ aryl group, optionally substituted 5 to 10-membered heteroaryl group, optionally substituted C₃₋₁₀ cyclic alkyl group, optionally substituted 4 to 10-membered heterocyclic alkyl group;
R⁴ and R⁵ are each selected groups from the following groups that are optionally replaced or substituted with one or more (e.g., 1, 2, or 3) R³: H, C₁₋₆ alkyls, C₃₋₁₀ naphthenic alkyls, 4 to 8-membered heterocyclic alkyl groups, C₆₋₁₀ aryl groups, 5 to 10-member heteroaryl groups, C₃₋₁₀ cyclene groups, and 4 to 10-member heterocyclic alkyl groups; alternatively, R⁴ and R bind to their attached nitrogen atoms to form an optionally substituted or selected ring from the following group of R³s that are substituted by one or more (e.g., 1, 2, or 3) R³s: 4 to 10 heterocyclic alkyl groups, 4 to 10 members heterocyclic alkyl groups, or 5 to 10 member heteroaryl groups;
R³ are independently selected from the group consisting of: R, —OR³¹, —C₁₋₄ alky-OR³¹, —N(R³²)R³³, —C₁₋₄ alky-N(R³¹)R³²;
R³¹ is selected from the group consisting of: H, optionally substituted C₁₋₄ alkyl groups, R³⁴, —C₁₋₄ alkyl —R³⁴;
R³² is selected from the group consisting of: H, optionally substituted C₁₋₄ alkyl groups;
R³³ is selected from the group consisting of: H, optionally substituted C₁₋₄ alkyl groups, R³⁴, —C₁₋₄ alkyl —R³⁴;
R³⁴ is selected from the group consisting of: C₃₋₁₀ naphthenyl, 4 to 8 members heterocyclic alkyl groups, C₆₋₁₀ aryl groups, 5 to 10 membered heteroaryl groups, C₃₋₁₀ cyclene groups, 4 to 10 membered heterocyclic alkyl groups; wherein, the naphthenic alkyl group, the aryl group, the heteroaryl group, the cyclic alkene group and the heterocyclic alkyl group are optionally substituted with one or more groups selected from the following group: —NH2, R;
R^{D} is independently selected from the group consisting of: H, optionally substituted C₁₋₄ alkyl groups; Alternatively, the two R^{D}s and the carbon atoms attached to them together form groups selected from the group consisting of: optionally substituted C₃₋₁₀ naphthenic alkyl groups, optionally substituted 4 to 10-membered heteronaphthenic alkyl groups;
W² is selected from the following group: —O—, —S—, —N(R^{s1})—;
Ring C is optionally substituted with the following groups: C₆₋₁₀ aryl groups, and 5 to 10 meta heteroaryl groups;
m3=0, 1, 2, 3 or 4;
each R^{C} is R^{C1} or R^{s1} independently;
each R^{C1} is independently selected from the group consisting of: halogens, optionally substituted C₁₋₆ alkyl groups, optionally substituted C₁₋₆ halogenated alkyl groups, hydroxyl groups and optionally substituted C₁₋₆ alkoxy groups, and optionally substituted C₁₋₆ halogenated alkoxy groups;
Ring B is the ring selected from the following group of alternatives: C₃₋₁₂ naphthenic alkyl group, 4 to 12 metaphthenic alkyl groups, C₃₋₁₀ cyclene groups, 4 to 10 membered heteronaphthenic alkyl groups;
each R^{B} is independently R^{B1}, R^{s1} or R^{s2};
each R^{B1} is independently selected from the group consisting of: halogens, hydroxyl groups, cyanos, optionally substituted C₁₋₆ alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₃₋₁₀ cyclic alkyl groups, optionally substituted 4 to 10-membered heterocyclic alkyl groups, optionally substituted C₆₋₁₀ aryl groups, and optionally substituted 5 to 10-membered heteroaryl groups;
m2=0, 1, 2, 3 or 4;
L¹ is —X—(W¹)ₙ₁—;
X is selected from the group consisting of: —C(O)—, —S(O)— and —S(O)₂—;
each W¹ is independently selected from the group consisting of: none, —O—, —S—, —C(O)—, —S(O)—, —S(O)₂—, —N(R¹)—, —N(R^{s1})—, —CH(R⁸)—, —C(R^{s2})₂—;
subscript n1=0, 1, 2 or 3;
Ring A is optionally substituted with the following group: C₆₋₁₀ aryl; 5 to 10 membered heteroaryl groups; C₆₋₁₀ aryl groups substituted by C₃₋₁₀ naphthenic alkyl groups, 4 to 10 meta heterocyclic alkyl groups, C₃₋₁₀ cyclene groups, 4 to 10 membered heterocyclic alkyl groups, C₆₋₁₀ aryl groups or 5 to 10 membered heteroaryl groups; 5 to 10 membered heteroaryl groups substituted with C₃₋₁₀ naphthenic alkyl groups, 4 to 10 metaphnocyclic alkyl groups, C₃₋₁₀ cyclene groups, 4 to 10 membered heterocyclic alkyl groups, C₆₋₁₀ aryl groups or 5 to 10 membered heteroaryl groups; C₆₋₁₀ aryl fused with C₃₋₁₀ naphthenyl, 4 to 10 heteronaphthenic alkyl groups, C₃₋₁₀ cyclene groups, 4 to 10 members heterocyclic alkenyl groups, C₆₋₁₀ aryl groups or 5 to 10 member heteroaryl groups; 5 to 10 membered heteroaryl groups fused with C₃₋₁₀ naphthenyl, 4 to 10 metaphthenic alkyl groups, C₃₋₁₀ cyclene groups, 4 to 10 membered heterocyclic alkyl groups, C₆₋₁₀ aryl groups, or 5 to 10 membered heteroaryl groups;
m1=0, 1, 2, 3 or 4;
each R^{A} is R^{A1}, R^{s1} or R^{s2} independently;
each R^{A1} is independently selected from the following groups: halogens, optionally substituted C₁₋₆ alkyl groups, optionally substituted C₁₋₆ halogenated alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₆₋₁₀ aryl groups, optionally replaced 5 to 10 membered heteroaryl groups; Alternatively, when two R^{A}R^{A1}s are located on adjacent ring atoms, the two R^{A}R^{A1}s and their adjacent ring atoms together form a selected ring from the following group: optionally substituted C₃₋₁₀ naphthenic alkyl group, optionally substituted 4 to 10-membered heterocyclic alkyl group, optionally substituted C₃₋₁₀ cyclic alkyl group, optionally substituted 4 to 10-membered heterocyclic alkene group, optionally substituted C₆₋₁₀ aryl group, optionally substituted C₆₋₁₀ aryl group, optionally replace 5 to 10 membered heteroaryl groups;
each R¹ and R⁸ are independently selected from the group consisting of: H, optionally substituted C₁₋₆ alkyl groups, optionally substituted C₃₋₆ naphthenic alkyl groups, halogens, optionally substituted C₁₋₆ halogenated alkyl groups, optionally substituted C₁₋₆ halogenated alkoxy groups (—O—C₁₋₆ halogenated alkyl groups), optionally substituted C₁₋₆ alkyl groups-O—C₁₋₆ alkyl groups, optionally substituted C₁₋₆ halogenated alkyl groups, Optionally substituted C₁₋₆ halogenated alkyl group-S—C₁₋₆ alkyl group, optionally substituted C₁₋₆ aminoalkyl group, optionally substituted C₃₋₁₀ naphthenic alkyl group, optionally substituted 4 to 10-membered heterocyclic alkyl group, optionally substituted C₆₋₁₀ aryl group, optionally substituted 5 to 10-membered heteroaryl group, optionally substituted C₃₋₁₀ halocyclic alkyl group, optionally substituted C₃₋₁₀ naphthenic alkyl group, optionally substituted 4 to 10-membered heterocyclic alkyl-C₁₋₄ alkyl groups, optionally substituted C₆₋₁₀ aryl-C₁₋₄ alkyl groups, optionally substituted 5 to 10-membered heteroaryl-C₁₋₄ alkyl groups, optionally substituted C₃₋₁₀ cyclic alkyl groups-C₁₋₄ alkyl groups, optionally substituted 4 to 10-membered heterocyclic alkyl groups-C₁₋₄ alkyl groups; Alternatively, R¹ or R⁸ and R^{s1} or R^{s2} on ring A form optionally substituted C₄₋₁₀ naphthenic or 4-10 heterocyclic alkyl groups;
R⁷ is optionally substituted groups from the following groups: none, C₁₋₆ alkyl groups, C₃₋₁₀ naphthenyl groups, 4 to 10 members heterocyclic alkyl groups, C₃₋₁₀ cyclic alkyl groups, 4 to 10 members heterocyclic alkyl groups, C₆₋₁₀ aryl groups, and 5 to 10 member heteroaryl groups.
R^{s1} is independently selected from the group consisting of: H, optionally substituted C₁₋₄ alkyl group, optionally substituted C₃₋₆ naphthenic group, and optionally substituted 4 to 6-membered heterocyclic group;
R^{s2} are independently selected from the group consisting of: H, optionally substituted C₁₋₄ alkyl groups, optionally substituted C₃₋₆ naphthenic alkyl groups, and optionally substituted 4 to 6-membered heterocyclic groups; or two R^{s2} and the carbon atoms attached to them together form an optionally substituted C₃₋₆ naphthenic group or an optionally substituted 4 to 6-membered heterocyclic group;
unless otherwise defined, said optional substitution means an unsubstituted or group of one or more (e.g., 1, 2, 3, or 4) hydrogen substituted by an R substituent;
R are independently selected from the group consisting of: D, halogens, C₁₋₆ alkyls, C₁₋₆ halogenated alkyls, C₁₋₆ hydroxyalkyls, C₂₋₆ alkyl groups, C₂₋₆ alkyl groups, —CN, —OR′, —NO2, —NR′R″, —SR′, OC(O)R′, —C(O)R′, —CO2R′, —CONR′, —OC(O)NR′R″, —NR″C(O)R′, —NR″—C(O)NR′R″, —NR′C(O)₂R′, —S(O)R′, —S(O)₂R′, —S(O)₂NR′_{R}″, NR″S(O)₂R′, any C₃₋₁₀ naphthenic alkyl group substituted with one or more R′, any 4 to 10 member heterocycloalkyl group substituted with one or more R′, any C₆₋₁₀ aryl group substituted with one or more R′, any 5 to 10 member heteroaryl group substituted with one or more R′, any —C₁₋₄ alkyl group C₃₋₁₀ naphthenic group substituted with one or more R″, any —C₁₋₄ alkyl group substituted with one or more R″, choose —C₁₋₄ alkyl-C₆₋₁₀ aryl group substituted with one or more R″″, choose —C₁₋₄ alkyl-5 to 10 aryl group substituted with one or more R″s;
each R′ is independently selected from the following groups: H, D, C₁₋₆ alkyls, C₁₋₆ halogenated alkyls, any C₃₋₁₀ naphthenic alkyl group substituted with one or more R″s, any 4 to 10 heterocyclic alkyl group substituted with one or more R″s, any C₆₋₁₀ aryl group substituted with one or more R″s, any 5 to 10 membered heteroaryl groups substituted with one or more R″s, any —C₁₋₄ alkyl group substituted with one or more R′s-C₃₋₁₀ naphthenic alkyl groups, choose —C₁₋₄ alkyl-4 to 10-membered heterocyclic alkyl groups substituted with one or more R″″, choose —C₁₋₄ alkyl —C₆₋₁₀ aryl group substituted with one or more R″, choose —C₁₋₄ alkyl-5 to 10-member heteroaryl group substituted with one or more R″s;
each R″ is selected from the group consisting of: H, D, C₁₋₄ alkyls, C₁₋₄ halogenated alkyls, and C₃₋₄ naphthenic groups;
each R″ is independently selected from the group consisting of: D, halogen, hydroxyl group, nitro, CN, C₁₋₆ alkyl group, C₁₋₆ halogenated alkyl group; or
the compound is of Formula II 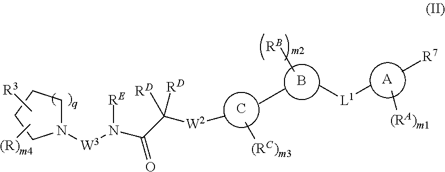
wherein
q is 0, 1, 2 or 3;
R³ is selected from the group consisting of: H, —OR³¹, —C₁₋₄ alky-OR³¹, —N(R³²)R³³, —C₁₋₄ alky-N(R³¹) R³²;
m4 is 0, 1, 2, 3, 4, 5, 6 or 7;
R³¹ is selected from the group consisting of: H, optionally substituted C₁₋₄ alkyl groups, R³⁴, —C₁₋₄ alkyl —R³⁴;
R³² is selected from the following group: H, optionally substituted C₁₋₄ alkyl groups;
R³³ is selected from the group consisting of: H, optionally substituted C₁₋₄ alkyl groups, R³⁴, —C₁₋₄ alkyl —R³⁴;
R³⁴ is selected from the group consisting of: C₃₋₁₀ naphthenyl, 4 to 8 members heterocyclic alkyl groups, C₆₋₁₀ aryl groups, 5 to 10 membered heteroaryl groups, C₃₋₁₀ cyclene groups, 4 to 10 membered heterocyclic alkyl groups; wherein, the naphthenic alkyl group, the aryl group, the heteroaryl group, the cyclic alkene group and the heterocyclic alkyl group are optionally substituted with one or more groups selected from the following group: —NH2, R;
W³ is selected from the group consisting of: —C(O)—, —S(O)—, —S(O)₂—, —C(R^{F}R^{F})₂—C(O)—, —C(R^{F}R^{F})₂—S(O)—, —C(R^{F}R^{F})₂—S(O)₂—;
R^{F} is independently selected from the group consisting of: H, optionally substituted C₁₋₄ alkyl groups, optionally substituted C₃₋₁₀ naphthenic alkyl groups, optionally substituted 4 to 10 membered heteronaphthenic alkyl groups; or two R^{F}R^{F}s and carbon atoms connected to them together form an optionally substituted C₃₋₆ naphthenic group or an optionally substituted 4 to 6-membered heterocyclic group;
R^{E} is selected from the group consisting of: H, optionally substituted C₁₋₄ alkyl groups, optionally substituted C₃₋₁₀ naphthenic alkyl groups, optionally substituted 4 to 10 membered heteronaphthenic alkyl groups;
R^{D} is independently selected from the group consisting of: H, optionally substituted C₁₋₄ alkyl groups; Alternatively, the two R^{D}s and the carbon atoms attached to them together form groups selected from the group consisting of: optionally substituted C₃₋₁₀ naphthenic alkyl groups, optionally substituted 4 to 10-membered heteronaphthenic alkyl groups;
W² is selected from the following group: —O—, —S—, —N(R^{s1})—;
Ring C is optionally substituted with the following groups: C₆₋₁₀ aryl groups, and 5 to 10 membered heteroaryl groups;
m3=0, 1, 2, 3 or 4;
each R^{C} is R^{C1} or R^{s1} independently;
each R^{C1} is independently selected from the group consisting of: halogens, optionally substituted C₁₋₆ alkyl groups, optionally substituted C₁₋₆ halogenated alkyl groups, hydroxyl groups and optionally substituted C₁₋₆ alkoxy groups, and optionally substituted C₁₋₆ halogenated alkoxy groups;
Ring B is the ring selected from the following group of alternatives: C₃₋₁₂ naphthenic alkyl group, 4 to 12 metaphthenic alkyl groups, C₃₋₁₀ cyclene groups, 4 to 10 membered heteronaphthenic alkyl groups;
each R^{B} is R^{B1}, R^{s1} or R^{s2} independently;
each R^{B1} is independently selected from the group consisting of: halogens, hydroxyl groups, cyanos, optionally substituted C₁₋₆ alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₃₋₁₀ cyclic alkyl groups, optionally substituted 4 to 10-membered heterocyclic alkyl groups, optionally substituted C₆₋₁₀ aryl groups, and optionally substituted 5 to 10-membered heteroaryl groups;
m2=0, 1, 2, 3 or 4;
L¹ is —X—(W¹)ₙ₁—;
X is selected from the group consisting of: —C(O)—, —S(O)— and —S(O)₂—;
each W¹ is independently selected from the group consisting of: none, —O—, —S—, —C(O)—, —S(O)—, —S(O)—, —N(R^{s1})—, —C(R^{s2})₂—;
subscript n1=0, 1, 2 or 3;
Ring A is optionally substituted with the following group: C₆₋₁₀ aryl; 5 to 10 membered heteroaryl groups; C₆₋₁₀ aryl groups substituted by C₃₋₁₀ naphthenic alkyl groups, 4 to 10 meta heterocyclic alkyl groups, C₃₋₁₀ cyclene groups, 4 to 10 membered heterocyclic alkyl groups, C₆₋₁₀ aryl groups or 5 to 10 membered heteroaryl groups; 5 to 10 membered heteroaryl groups substituted with C₃₋₁₀ naphthenic alkyl groups, 4 to 10 metaphnocyclic alkyl groups, C₃₋₁₀ cyclene groups, 4 to 10 membered heterocyclic alkyl groups, C₆₋₁₀ aryl groups or 5 to 10 membered heteroaryl groups: C₆₋₁₀ aryl fused with C₃₋₁₀ naphthenyl, 4 to 10 heteronaphthenic alkyl groups, C₃₋₁₀ cyclene groups, 4 to 10 members heterocyclic alkenyl groups, C₆10 aryl groups or 5 to 10 member heteroaryl groups; 5 to 10 membered heteroaryl groups fused with C₃₋₁₀ naphthenyl, 4 to 10 metaphthenic alkyl groups, C₃₋₁₀ cyclene groups, 4 to 10 membered heterocyclic alkyl groups. C₆₋₁₀ aryl groups, or 5 to 10 membered heteroaryl groups;
m1=0, 1, 2, 3 or 4;
each R^{A} is R^{A1}, R^{s1} or R^{s2} independently;
each R^{A1} is independently selected from the group consisting of: halogens, optionally substituted C₁₋₆ alkyl groups, optionally substituted C₁₋₆ halogenated alkyl groups, optionally substituted C₁₋₆ alkoxy groups, optionally substituted C₁₋₆ alkyl groups, optionally substituted C₃₋₁₀ naphthenic alkyl groups, optionally substituted 4 to 10-membered heterocyclic alkyl groups, optionally substituted C₆₋₁₀ aryl groups, optionally replaced 5 to 10 membered heteroaryl groups; Alternatively, when two R^{A1}s are located on adjacent ring atoms, the two R^{A1}s and their adjacent ring atoms together form a selected ring from the following group: optionally substituted C₃₋₁₀ naphthenic alkyl group, optionally substituted 4 to 10-membered heterocyclic alkyl group, optionally substituted C₃₋₁₀ cyclic alkyl group, optionally substituted 4 to 10-membered heterocyclic alkene group, optionally substituted C₆₋₁₀ aryl group, optionally substituted C₆₋₁₀ aryl group, optionally replace 5 to 10 membered heteroaryl groups;
R⁷ is optionally substituted groups from the following groups: none, C₁₋₆ alkyl groups, C₃₋₁₀ naphthenyl groups, 4 to 10 members heterocyclic alkyl groups, C₃₋₁₀ cyclic alkyl groups, 4 to 10 members heterocyclic alkyl groups, C₆₋₁₀ aryl groups, and 5 to 10 member heteroaryl groups.
R^{s1} is independently selected from the group consisting of: H, optionally substituted C₁₋₄ alkyl group, optionally substituted C₃₋₆ naphthenic group, and optionally substituted 4 to 6-membered heterocyclic group;
R^{s2} are independently selected from the group consisting of: H, optionally substituted C₁₋₄ alkyl groups, optionally substituted C₃₋₆ naphthenic alkyl groups, and optionally substituted 4 to 6-membered heterocyclic groups; or two R^{s2} and the carbon atoms attached to them together form an optionally substituted C₃₋₆ naphthenic group or an optionally substituted 4 to 6-membered heterocyclic group;
unless otherwise defined, said optional substitution means an unsubstituted or group of one or more (e.g., 1, 2, 3, or 4) hydrogen substituted by an R substituent;
R are independently selected from the group consisting of: D, halogens, C₁₋₆ alkyls, C₁₋₆ halogenated alkyls, C₁₋₆ hydroxyalkyls, C₂₋₆ alkyl groups, C₂₋₆ alkyl groups, —CN, —OR′, —NO2, —NR′R″, —SR′, OC(O)R′, —C(O)R′, —CO2R′, —CONR′, —OC(O)NR′R″, —NR″C(O)R′, —NR″—C(O)NR′R″, —NR′C (O)₂R′, —S(O)R′, —S(O)₂R′, —S(O)₂NR′R″, NR″S(O)₂R′, any C₃₋₁₀ naphthenic alkyl group substituted with one or more R′, any 4 to 10 member heterocycloalkyl group substituted with one or more R′, any C₆₋₁₀ aryl group substituted with one or more R′, any 5 to 10 member heteroaryl group substituted with one or more R′, any —C₁4 alkyl group C₃₋₁₀ naphthenic group substituted with one or more R″, any —C₁₋₄ alkyl group substituted with one or more R″, choose —C₁₋₄ alkyl-C₆₋₁₀ aryl group substituted with one or more R″″, choose —C₁₋₄ alkyl-5 to 10 aryl group substituted with one or more R″s;
each R′ is independently selected from the group consisting of H, D, C₁₋₆ alkyls, C₁, halogenated alkyls, any C₃₋₁₀ naphthenic alkyl group substituted with one or more R″, any 4 to 10 heterocyclic alkyl group substituted with one or more R″s, any C₆₋₁₀ aryl group substituted with one or more R″s, any 5 to 10 membered heteroaryl groups substituted with one or more R″, any —C₁₋₄ alkyl group substituted with one or more R′s-C₃₋₁₀ naphthenic alkyl groups, choose —C₁₋₄ alkyl-4 to 10-membered heterocyclic alkyl groups substituted with one or more R″″, choose —C₁₋₄ alkyl —C₆₋₁₀ aryl group substituted with one or more R″, choose —C₁₋₄ alkyl-5 to 10-member heteroaryl group substituted with one or more R″s;
each R″ is selected from the group consisting of H, D, C₁₋₄ alkyls, C₁₋₄ halogenated alkyls, and C₃₋₄ naphthenic groups;
each R″ is independently selected from the group consisting of D, halogen, hydroxyl group, nitro, CN, C₁₋₆ alkyl group, C₁₋₆ halogenated alkyl group.

2. The compound of claim 1, wherein the compound is shown in Formula I-1 or Formula I-2

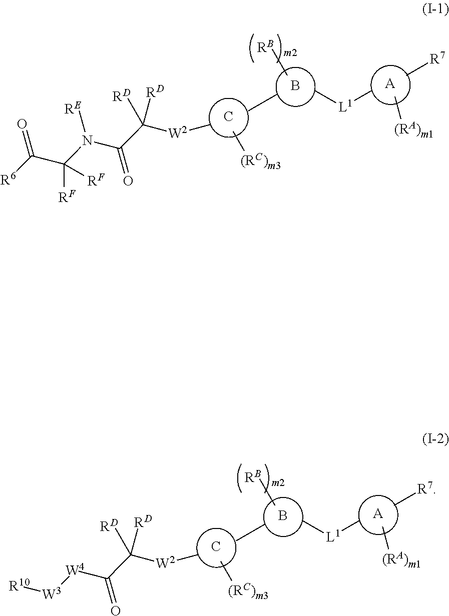

3. The compound of claim 1, wherein the
(i) Ring B is 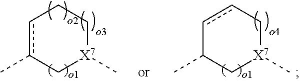
wherein, 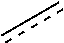
is a single or double bond, X⁷ is N or CH, o1 is 1 or 2 o2 is 0 1 2 or 3, o3 is 0 or 1, o4 is 0, 1, 2 or 3; and/or
(ii) Ring C is 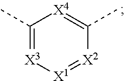
wherein X¹, X², X³ and X⁴ are independently selected from the group consisting of: CH and N.

4. The compound of claim 1, wherein R⁶ is —NR⁴R⁵ and —NR⁴R⁵ is a 4 to 10-membered heterocyclic alkyl group substituted with one or more R³s; Alternatively, W⁴ is —N(R)—; W³ is selected from the following group: —C(O)—, —S(O)—, —S(O)₂—, R¹0 is unsubstituted or substituted with one or more R^{H} groups selected from the lower group: C₆₋₁₀ aryl group, and 5 to 10 membered heteroaryl groups.

5. The compound of claim 1 is characterized by the fact that the compound shown has one or more of the following features:
(i) Ring A is the ring selected from the following group: 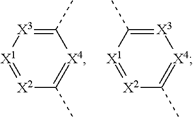
wherein X¹, X², X³ and X⁴ are independently selected from the group consisting of: CH and N
(ii) R⁷ is selected from the following group: 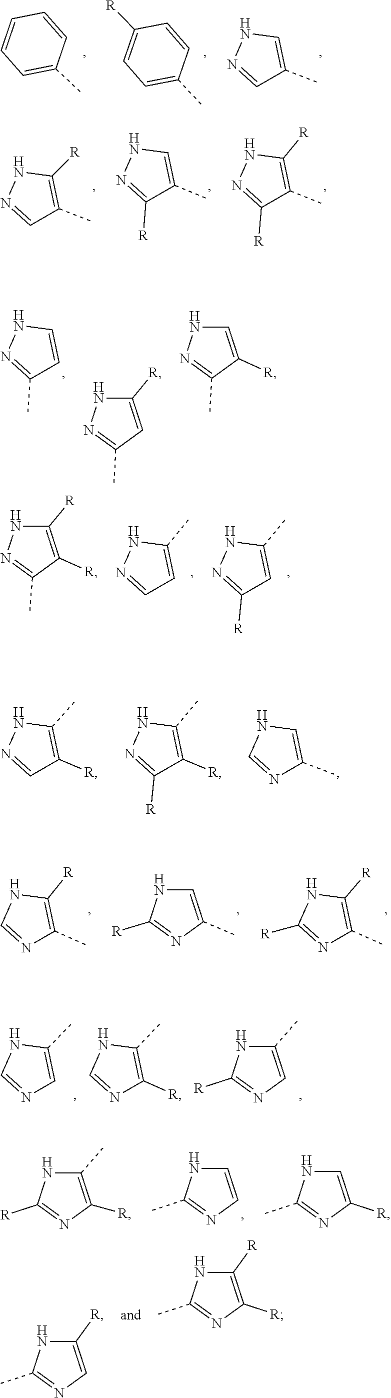
(iii) in R⁷, R is independently selected from the group consisting of: C₁₋₆ alkyl and —NR′R″; wherein each R′ is independently selected from the group consisting of: H, C₁₋₆ alkyl groups; Each R″ is selected from the following group: H, C₁₋₄ alkyls.

6. The compound of claim 1, wherein the compound is selected from Tables A-I, Table B-I and Table C-I.

7. (canceled)

8. The compound of claim 1, wherein the compound is of Formula II-1 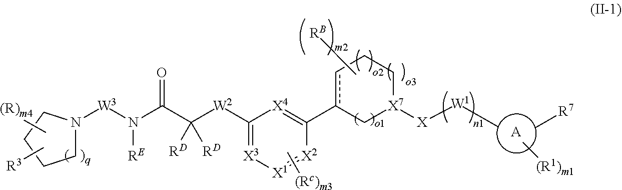
wherein [symbol]
is single or double bond; X⁷ is N or CH; o1 is 1 or 2, o2 is 0, 1, 2 or 3, o3 is 0 or 1;
X¹, X², X³ and X⁴ are independently selected from the group consisting of: CH and N; or
the compound is of Formula II-2 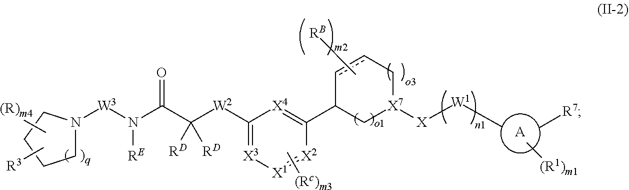
wherein [symbol]
is single or double bond; X⁷ is N or CH; o1 is 1 or 2, o4 is 0, 1, 2 or 3;
X¹, X², X³ and X⁴ are independently selected from the group consisting of: CH and N.

9. The compound of claim 1, wherein, Wherein 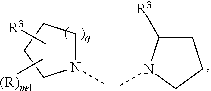
R³ is —N(R³²)R³³ or —C₁₋₄ alkylo-N(R³¹)R³²; R³² and R³³ are independently selected from the group consisting of: H, C₁₋₄ alkyls.

10. The compound of claim 1, wherein the compound has one or more of the following features:
(a) R⁷ is selected from the following group: 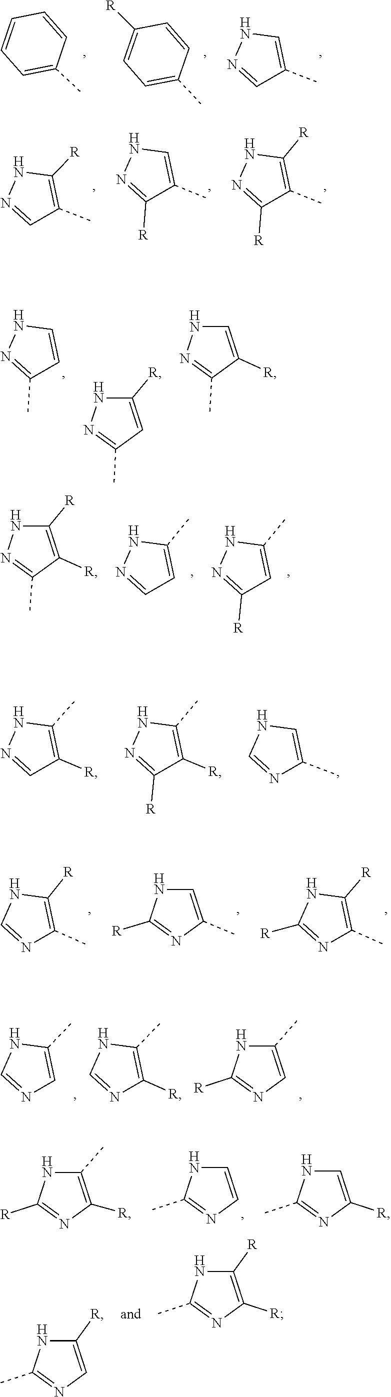
(b) in R⁷, R is independently selected from the group consisting of: C₁₋₆ alkyl and —NR′R″; wherein each R′ is independently selected from the group consisting of: H, C₁₋₆ alkyl groups; Each R″ is selected from the group consisting of: H, C₁₋₄ alkyl groups;
(c) Ring A is the ring selected from the following group: 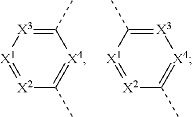
wherein X¹, X², X³ and X⁴ are independently selected from the group consisting of: CH and N.
(d) R^{A}s are H, C₁₋₄ alkyl or R^{A1} independently; and R^{A1} is selected from the group consisting of: halogens, optionally substituted C₁₋₆ alkyl groups, optionally substituted C₁₋₆ halogenated alkyl groups, and optionally substituted C₁₋₆ alkoxy groups;
(e) W² is —O— or —N(Rs)-.

11. The compound of claim 1, wherein the compound is selected from Table A-II, Table B-II and C-II.

12. A drug composition, wherein comprising:
(i) the compound of claim 1 or the pharmaceutically acceptable salt thereof, or the isomer, solvate, crystal form, or prodrug thereof, and
(ii) pharmaceutically acceptable carriers or excipients.

13. A method for the treatment or prevention of diseases related to BCL9/β-catenin interaction, comprising administering the compound of claim 1 or the pharmaceutically acceptable salt thereof, or the isomer, solvate, crystal form, or prodrug thereof.

14. The method of claim 14, wherein the diseases related to the interaction of BCL9/β-catenin include: cancer, tumor, or a combination thereof.

15. A method for the treatment or prevention of fibrosis or related diseases, comprising administering the compound of claim 1 or the pharmaceutically acceptable salt thereof, or the isomer, solvate, crystal form, or prodrug thereof in the preparation of a drug.
